(19) United States
(12) Patent Application Publication
(10) Pub. No.: US 2026/0061168 A1
(43) Pub. Date: Mar. 5, 2026

(54) LEAD SCREW DRIVEN SHEATH DILATOR

(71) Applicant: Edwards Lifesciences Corporation, Irvine, CA (US)

(72) Inventor: Michael C. Murad, Lake Mathews, CA (US)

(73) Assignee: Edwards Lifesciences Corporation, Irvine, CA (US)

(21) Appl. No.: 19/385,522

(22) Filed: Nov. 11, 2025

Related U.S. Application Data
(63) Continuation of application No. PCT/US2024/029599, filed on May 16, 2024.
(60) Provisional application No. 63/502,907, filed on May 17, 2023.

(51) Int. Cl. A61M 25/06; A61M 25/00; A61M 29/00

(52) U.S. Cl. CPC A61M 25/0662; A61M 25/0023; A61M 25/0097; A61M 29/00; A61M 2025/0024; A61M 2025/0098

(57) ABSTRACT

Various implementations include a sheath system and corresponding dilator. The system includes a radially expandable sheath having an inner layer and a tubular strain relief layer, and a dilator sized and configured to be received within the lumen of the sheath.

### CROSS-REFERENCE TO RELATED APPLICATIONS

This application is a continuation of International Application No. PCT/US2024/029599, filed May 16, 2024, which claims the benefit of U.S. Provisional Application No. 63/502,907, filed May 17, 2023, the contents of which are incorporated herein by reference in its entirety.

### FIELD

The present application is directed to a sheath for use with catheter-based technologies for repairing and/or replacing heart valves, as well as for delivering an implant, such as a prosthetic valve to a heart via the patient's vasculature.

### BACKGROUND

Endovascular delivery catheter assemblies are used to implant prosthetic devices, such as a prosthetic valve, at locations inside the body that are not readily accessible by surgery or where access without invasive surgery is desirable. For example, aortic, mitral, tricuspid, and/or pulmonary prosthetic valves can be delivered to a treatment site using minimally invasive surgical techniques.

Percutaneous interventional medical procedures utilize the large blood vessels of the body reach target destinations rather than surgically opening target site. There are many types of diseases states that can be treated via interventional methods including coronary blockages, valve replacements (TAVR) and brain aneurysms. These techniques involve using wires, catheters, balloons, electrodes and other thin devices to travel down the length of the blood vessels from the access site to the target site. The devices have a proximal end which the clinician controls outside of the body and a distal end inside the body which is responsible for treating the disease state. Percutaneous interventional procedures offer several advantages over open surgical techniques. First, they require smaller incision sites which reduces scarring and bleeding as well as infection risk. Procedures are also less traumatic to the tissue, so recovery times are reduced. Finally, interventional techniques can usually be performed much faster, and with fewer clinicians participating in the procedure, so overall costs are lowered. In some cases, the need for anesthesia is also eliminated, further speeding up the recovery process and reducing risk.

A single procedure typically uses several different guidewires, catheters, and balloons to achieve the desired effect. One at a time, each tool is inserted and then removed from the access site sequentially. For example, a guidewire is used to track to the correct location within the body. Next a balloon may be used to dilate a section of narrowed blood vessel. Last, an implant may be delivered to the target site. Because catheters are frequently inserted and removed, introducer sheaths are used to protect the local anatomy and simplify the procedure.

An introducer sheath can be used to safely introduce a delivery apparatus into a patient's vasculature (e.g., the femoral artery). Introducer sheaths are conduits that seal onto the access site blood vessel to reduce bleeding and trauma to the vessel caused by catheters with rough edges. An introducer sheath generally has an elongated sleeve that is inserted into the vasculature and a housing that contains one or more sealing valves that allow a delivery apparatus to be placed in fluid communication with the vasculature with minimal blood loss. Once the introducer sheath is positioned within the vasculature, the shaft of the delivery apparatus is advanced through the sheath and into the vasculature, carrying the prosthetic device. Expandable introducer sheaths, formed of highly elastomeric materials, allow for the dilating of the vessel to be performed by the passing prosthetic device.

The expandable sheath, formed of highly elastomeric materials and some including one or more folds to aid in expansion, expands as an implantable device is inserted through the sheath. These sheaths sometimes include a strain relief portion that extends along/over the outer surface of the sheath (e.g., at the proximal end) and forms a smooth transition from the sheath hub to the sheath. The strain relief portion restricts expansion of the underlying sheath and helps to ensure hemostasis between the portions of the sheath inside the patient and the sheath hub (external to the patient). Because the strain relief portion resists expansion, higher push forces are required as the delivery device and implant are introduced into and advanced through the sheath/strain relief portion. In addition, recent trends in heart valves including thicker PVL skirts has increased the crimped profile of the heart valve/delivery device and can lead to even higher push forces through the sheath, and particularly the strain relief portion.

Accordingly, there remains a need for devices systems, and methods providing a sheath including a strain relief portion, that allows the strain relief portion to expand reducing the initial push force when introducing the delivery device and implant.

### SUMMARY

Implementations of the present expandable sheath system can minimize trauma to the vessel and damage to the sheath and prosthetic device by reducing push forces through the sheath. Some implementations ensure that the sheath is not damaged in an effort to dilate or expand the strain relief portion. Some implementations can comprise a sheath with a smaller profile than that of prior art introducer sheaths. Furthermore, certain implementations can reduce the length of time a procedure takes, as well as reduce the risk of a longitudinal or radial vessel tear, or plaque dislodgement because lower push force is required and only one sheath is used, rather than several different sizes of sheaths.

An implementation of the present disclosure is a sheath system that includes: a radially expandable sheath including an inner layer and a tubular strain relief layer provided over the inner layer that limits radial expansion of the sheath. The system also includes a dilator sized and configured to be received within the lumen of the sheath for expanding at least a portion of the inner layer and/or strain relief layer.

In some implementations, the present disclosure is direct to a dilator including a dilator shaft rotationally movable with respect to a knob/handle to adjust the length of the dilator extending beyond the knob.

In some implementations, the dilator includes a dilator shaft, a knob coupled to a proximal end of the dilator shaft; a dilator hub (e.g., coupler) rotatably coupled to the knob; and a pin coupling the dilator hub with the dilator shaft and preventing rotational movement therebetween, where rotational movement of the knob results in a corresponding axial movement of the dilator shaft in a direction along a longitudinal axis of the dilator.

In some implementations, rotational movement of the knob is used to adjust a length of the dilator shaft extending beyond the distal end of dilator hub and/or the distal end of the knob.

In some implementations, rotational movement of the knob in a first direction causes the dilator shaft to move axially in a first direction, and rotational movement of the knob in a second, opposite, direction, causes the dilator shaft to move axially in a second, opposite direction (e.g., rotational movement of the knob in a clockwise direction results in a corresponding distal axial movement of the dilator shaft, and rotational movement of the knob in a counterclockwise direction results in corresponding proximal movement of the dilator shaft).

In some implementations, the dilator shaft is moveable from a first position (retracted) to a second position (extended), where a length of the dilator shaft (L2) in the second position is greater than a length of the dilator shaft (L1) in the first position (e.g., where the length of the dilator shaft is measured along the elongated body portion between the distal end of the dilator hub and the proximal end of the tapered distal end).

In some implementations, the length of the dilator shaft (L2) in the second position ranges between 4 inches and 6 inches. In some implementations, the length of the dilator shaft (L2) in the second position is 5 inches.

In some implementations, the knob is threadingly coupled to the proximal end of the dilator shaft such that rotational movement between the knob and the dilator shaft results in the corresponding axial movement of the dilator shaft.

In some implementations, the knob includes a threaded central lumen extending at least partially therethrough (e.g., the threaded central lumen extends from the proximal end to a distal end of the knob), and the dilator shaft includes a threaded outer surface received within and threadingly coupled to threaded central lumen of the knob.

In some implementations, a length of travel of the dilator shaft within the threaded central lumen of the knob corresponds to a screw travel length (L3), where the screw travel length is defined between a proximal end of the knob and a location within the threaded central lumen where the dilator shaft is in the second position. In some implementations, when the dilator shaft is in second position at least four threads of the dilator shaft are engaged with the threaded central lumen.

In some implementations, the screw travel length ranges between 1 inch and 3 inches. In some implementations, the screw travel length is 2 inches.

In some implementations, the dilator shaft includes an increased diameter portion adjacent the proximal end, where the threaded outer surfaced provided along at least a portion of the increased diameter portion.

In some implementations, the dilator shaft includes an elongated body portion extending between the increased diameter portion and the distal end of the dilator shaft, where the increased diameter portion has a diameter (D1) greater than a diameter (D2) of the elongated body portion.

In some implementations, the diameter (D2) of the elongated body portion ranges from 16 French to 28 French. In some implementations, the diameter (D2) of the elongated body portion is 22 French. In some implementations, the dilator shaft includes an expansion element projecting from the outer surface of the dilator shaft, the expansion element can include a regular or irregular shaped projection extending from the outer surface (e.g., around all or a portion of the circumference) of the dilator shaft, the diameter of the expansion element is 22 French. In some implementations, the expansion element of dilator has a diameter ranging from 12 French to 24 French, from 14 French to 24 French, from 14 French to 22 French.

In some implementations, the dilator shaft includes a tapered distal end. In some implementations, the dilator shaft includes a decreasing taper toward the distal end of the dilator shaft) extending from the distal end of the dilator shaft to the elongated body portion.

In some implementations, the tapered distal end tapers from the diameter of the elongated body portion (D2) to a distal end diameter (D3), where the distal end diameter (D3) less than the diameter of the elongated body portion (D2).

In some implementations, the tapered distal end includes a concave tapered. In some implementations, the tapered distal end includes a smooth tapering surface, a convex surface tapering surface, and/or any other regular or irregularly shaped tapered surface).

In some implementations, the dilator shaft extends through a central lumen extending through the dilator hub. In some implementations, the central lumen extends from the proximal end to the distal end of the dilator hub.

In some implementations, the dilator hub is rotatably coupled to the knob. In some implementations, the dilator hub can freely rotate with respect to the knob.

In some implementations, the dilator hub includes a clip for rotatably coupling the dilator hub to the knob.

In some implementations, the dilator hub includes a shoulder extending radially inward from the central lumen, the shoulder is received within a corresponding recess provided on the knob. In some implementations, the shoulder freely rotates within the recess.

In some implementations, the central lumen of the dilator hub includes a first diameter portion and a second diameter portion, where a diameter of the first diameter portion 460 is greater than a diameter of the second diameter portion, and the shoulder is provided on the first diameter portion (e.g., adjacent a proximal end of the dilator hub).

In some implementations, the recess is provided on a projection extending axially from a distal end of the knob.

In some implementations, the projection includes a tapered outer surface configured to assist with advancing the projection beyond the shoulder and securing the shoulder within the recess. In some implementations, the projection includes a reducing tapered outer surface.

In some implementations, an axial length of the first diameter portion corresponds to at least a length of the projection.

In some implementations, the central lumen of the dilator hub includes a third diameter portion, where a diameter of the third diameter portion is less than a diameter of the second diameter portion.

In some implementations, the diameter of the second diameter portion is greater than the diameter of the increased diameter portion (e.g., the diameter of the threaded outer surface) of the dilator shaft such that the dilator shaft is axially movable within the second diameter portion, and the third diameter portion forms a second shoulder within the central lumen of the dilator hub such that interference between the second shoulder and the distal end of the increase diameter portion prevents axial movement of the dilator shaft within the dilator hub.

In some implementations, the dilator hub includes a locking channel.

In some implementations, the locking channel extends from the distal end of the dilator hub axially towards a proximal end of the dilator hub and circumferentially around the dilator hub.

In some implementations, the locking channel includes a guide portion that extends at an angle between an opening on a distal end surface of the dilator hub and a locking portion that extends circumferentially around the dilator hub.

In some implementations, the guide portion is sized and configured to direct a corresponding projection (e.g., guide provided on the locking sleeve) in an axial direction along a side wall of the guide portion towards the locking portion upon rotation of the dilator hub and/or the rotation of sheath locking sleeve, where the locking portion of the locking channel is configured to securely engage the corresponding projection (e.g., guide) and fix an axial position of the dilator hub with respect to the sheath locking sleeve/sheath hub.

In some implementations, the locking portion includes a catch extending from a sidewall of the locking portion for securing the guide within the locking portion of the locking channel.

In some implementations, the dilator shaft includes an elongated slot extending radially through at least a portion of the dilator shaft, where the slot extends axially along a portion of a length of the dilator shaft. In some implementations, the slot extends through the entire thickness/width of the dilator shaft, the slot being coplanar with the longitudinal axis of the dilator shaft.

In some implementations, the pin extends from a first side wall of the dilator hub, through the slot, and into a second sidewall of the dilator hub,
wherein engagement between the pin and the slot restricts rotational movement between the dilator hub and the dilator shaft.

In some implementations, the engagement between the pin and the slot limits axial movement of the dilator shaft with respect to the dilator hub. In some implementations, engagement between the proximal and distal ends of the slot with the pin limits movement of the dilator shaft with respect to the dilator hub along the longitudinal axis of the dilator shaft.

Further implementation of the present disclosure is directed to sheath system. The sheath system includes a radially expandable sheath comprising: a continuous inner layer defining a central lumen extending therethrough, the inner layer having at least one folded portion; and a tubular strain relief layer provided over and/or along the inner layer positioned at a proximal end of the sheath and extending along at least a portion of a length of the sheath. The sheath system further includes a dilator for expanding at least a portion of the sheath, the dilator including: a dilator shaft sized and configured to be received within the central lumen of the sheath; a knob coupled to a proximal end of the dilator shaft; a dilator hub (e.g., coupler) rotatably coupled to the knob; and a pin coupling the dilator hub and the dilator shaft and preventing rotational movement therebetween, where rotational movement of the knob results in a corresponding axial movement of the dilator shaft in a direction along a longitudinal axis of the dilator to adjust a length of the dilator shaft received within the central lumen of the sheath, and where at least a portion of the strain relief layer is configured to locally expand from an unexpanded configuration at a first diameter to an expanded configuration at a second, larger, diameter, and then locally contract at least partially back to the unexpanded configuration.

In some implementations, at least a portion of the strain relief layer is configured to locally expand from the unexpanded configuration to the expanded configuration in response to the outwardly directed radial force exerted against the central lumen (e.g., inner layer) by the dilator shaft (e.g., by the dilator shaft and/or a medical device against the central lumen), and then locally contract at least partially back to the unexpanded configuration as the dilator shaft (e.g., the dilator shaft and/or medical device) moves within the lumen.

In some implementations, the outer diameter of the dilator shaft is greater than an inner diameter of the sheath along a length corresponding to the strain relief layer, as such, movement of the dilator shaft within the central lumen of the sheath causes the sheath and the overlaying portion of the strain relief layer to radially expand.

In some implementations, at least a portion of the sheath is configured to locally expand from an unexpanded configuration in which the lumen has a first diameter to an expanded configuration in which the lumen has a second, larger, diameter in response to an outwardly directed radial force exerted on the lumen of the inner layer by the dilator shaft (e.g., the dilator shaft and/or a medical device) against the inner layer, and then locally contract at least partially back to the unexpanded configuration as the dilator shaft (e.g., the dilator shaft and/or medical device) passes through the lumen.

In some implementations, the outer diameter of the dilator shaft is greater than an inner diameter of the sheath, as such, movement of the dilator shaft within the central lumen of the sheath causes the sheath to radially expand.

In some implementations, rotational movement of the knob is used to adjust a length of the dilator shaft extending beyond the distal end of dilator hub and/or the distal end of the knob, and where the dilator shaft is moveable from a first position (retracted) to a second position (extended), where a length of the dilator shaft (L2) in the second position is greater than a length of the dilator shaft (L1) in the first position.

In some implementations, when the dilator shaft is received within the central lumen of the sheath and in the second position, the dilator shaft extends along a length of the sheath corresponding to the strain relief layer.

In some implementations, when the dilator shaft is received within the central lumen of the sheath and in the second position, the dilator shaft extends along a length of the sheath corresponding to a majority of the (length) strain relief layer.

In some implementations, when the dilator shaft is received within the central lumen of the sheath and in the second position, the dilator shaft extends along a length of the sheath corresponding to an entire length of the strain relief layer.

In some implementations, in the second position a distal end of the elongated body portion of the dilator shaft is aligned with a distal end of the strain relief layer. In some implementations, when the dilator shaft includes a tapered distal end and is in the second position with respect to the dilator hub, the distal end of the elongated body portion aligns with the distal end of the strain relief layer and the tapered distal end extends beyond the distal end of the strain relief layer, because the tapered distal end has a diameter less than the diameter of the elongated body portion, the tapered distal end does not expand the portion of the sheath beyond the strain relief layer.

In some implementations, the length of the dilator shaft in the second position corresponds to the entire length of the strain relief layer including a tolerance amount. In some implementations, the tolerance amount is +/−15 mm.

In some implementations, the strain relief layer comprises a stiffer and/or less elastomeric material than the inner layer and restricts expansion of the inner layer. In some implementations, the strain relief layer comprises a material having a higher durometer than the inner layer such that the strain relief layer restricts expansion of the sheath.

In some implementations, as the strain relief layer moves from the unexpanded to the expanded configuration, a length of the strain relief layer remains constant.

In some implementations, the dilator hub is rotatably coupled to the knob. In some implementations, the dilator hub can freely rotate with respect to the knob.

In some implementations, the dilator hub includes a locking channel sized and configured to couple the dilator hub (e.g., and dilator) to the sheath. In some implementations, the locking channel is used to couple the dilator hub to a sheath hub, via a locking sleeve. In some implementations, the locking channel is similar to locking channel provided on the hub body.

In some implementations, the locking channel includes a guide portion that extends between an opening on a distal end surface of the dilator hub and a locking portion of the locking channel, the locking portion extending in a direction circumferentially around the dilator hub. In some implementations, the guide portion extends at an angle between the opening on the distal end surface of the dilator hub and the locking portion of the locking channel.

In some implementations, the sheath system further includes a sheath hub fixedly coupled to the proximal end of the sheath, the sheath hub including a central lumen extending therethrough and coaxial with the lumen of the sheath, where the dilator shaft is sized and configured to be received within the central lumen of the sheath hub. In some implementations, the dilator shaft is slidably and/or rotatably received) within the central lumen of the sheath hub.

In some implementations, the sheath system further includes a sheath locking sleeve removably coupled to the sheath hub, the sheath locking sleeve comprising a sleeve body having a proximal end and a distal end and defining a central lumen extending longitudinally between the proximal end and the distal end, a guide disposed on an outer surface of the sleeve body, where the guide is movable within the locking channel between an unlocked position where the sheath locking sleeve is rotationally and axially movable with respect to the dilator hub, and a locked position where the sheath locking sleeve is axially fixed with respect to the dilator hub.

In some implementations, the guide portion of the locking channel is configured to direct the guide in an axial direction along a side wall of the guide portion towards the locking portion upon rotation of at least one of the dilator hub or the sheath locking sleeve, wherein the locking portion of the locking channel is configured to securely engage the guide fixing an axial position of the introducer dilator hub with respect to the sheath locking sleeve.

In some implementations, the sheath locking sleeve is securely couplable to a sheath hub, the sheath hub having an elongated body portion with a central lumen extending therethrough, where the sheath is coupled to a distal end of the body portion, where a central lumen of the sheath is aligned with the central lumens of the sheath hub, the sheath locking sleeve, and the dilator hub.

A further implementation of the present disclosure is directed to a method of adjusting the length of a dilator comprising: providing dilator comprising a dilator shaft, a knob coupled to a proximal end of the dilator shaft, a dilator hub (e.g., coupler) rotatably coupled to the knob, and a pin coupling the dilator hub and the dilator shaft and preventing rotational movement therebetween, and rotating the knob in a first direction causing the dilator shaft to engage the knob and resulting in a corresponding axial movement of the dilator shaft in a direction along a longitudinal axis of the dilator.

In some implementations, rotational movement of the knob in a second, opposite, direction, causes the dilator shaft to move axially in a second, opposite direction. In some implementations, rotational movement of the knob in a clockwise direction results in a corresponding distal axial movement of the dilator shaft, and rotational movement of the knob in a counterclockwise direction results in corresponding proximal movement of the dilator shaft.

In some implementations, rotating the knob results in the dilator shaft is moving from a first position (retracted) to a second position (extended), where a length of the dilator shaft (L2) in the second position is greater than a length of the dilator shaft (L1) in the first position. In some implementations, the length of the dilator shaft is measured along the elongated body portion between the distal end of the dilator hub and the proximal end of the tapered distal end.

In some implementations, the knob includes a threaded central lumen extending at least partially therethrough and the dilator shaft includes a threaded outer surface received within and threadingly coupled to threaded central lumen of the knob, where rotating the knob causes the threaded outer surface of the dilator shaft to threadingly engage the threaded central lumen of the knob resulting in a corresponding axial movement of the dilator shaft in a direction along the longitudinal axis of the dilator.

In some implementations, the pin extends from the dilator hub through a slot extending radially through the dilator shaft, where engagement between the pin and the slot restricts rotational movement between the dilator hub and the dilator shaft, where rotating the knob causes the pin to slidingly engage the slot guiding axial movement of the dilator shaft, and limiting rotational movement of the dilator shaft with respect to the dilator hub.

In some implementations, the dilator hub is rotatably coupled to the knob such that the dilator hub can freely rotate with respect to the knob, wherein an axial location of the dilator hub with respect to the knob is fixed during rotation of the knob.

Another implementation of the present disclosure is directed to a method of dilating a sheath including providing a radially expandable sheath including: a continuous inner layer defining a central lumen therethrough, the inner layer having at least one folded portion; and tubular strain relief layer provided over the inner layer positioned at a proximal end of the sheath and extending along at least a portion of a length of the sheath, wherein at least a portion of the strain relief layer is configured to locally expand from an unexpanded configuration at a first diameter to an expanded configuration at a second, larger, diameter in response to an outwardly directed radial force exerted on the central lumen (e.g., by the dilator shaft received within the lumen of the inner layer), and then locally contract at least partially back to the unexpanded configuration as the outwardly directed radial force is removed from the central lumen (e.g., as the dilator moves within the lumen). The method further including providing a dilator for expanding at least a portion of the sheath, the dilator including: a dilator shaft sized and configured to be received within the central lumen of the expandable sheath; a knob coupled to a proximal end of the dilator shaft; a dilator hub (e.g., coupler) rotatably coupled to the knob; and a pin coupling the dilator hub and the dilator shaft and preventing rotational movement therebetween. The method further including: coupling the dilator to the sheath; rotating the knob in a first direction causing the dilator shaft to engage the knob and resulting in a corresponding axial movement of the dilator shaft in a distal direction along a longitudinal axis of the dilator thereby increasing a length of the dilator shaft received within the central lumen of the sheath; advancing the dilator shaft through a portion of the central lumen of the sheath corresponding to the strain relief layer such that the dilator shaft exerts an outwardly directed radial force against the central lumen and causes the inner layer and the strain relief layer proximate the dilator shaft to locally expand from an unexpanded configuration to an expanded configuration; rotating the knob in a second direction opposite to the first direction, causing the dilator shaft to engage the knob and resulting in a corresponding axial movement of the dilator shaft in a proximal direction along the longitudinal axis of the dilator thereby reducing a length of the dilator shaft received within the central lumen of the sheath and removing the outwardly directed radial force exerted on the central lumen thereby causing the inner layer and the strain relief layer to locally contract at least partially back to the unexpanded configuration; uncoupling the dilator from the sheath; and removing the dilator from the sheath.

In some implementations, the dilator hub includes a locking channel sized and configured to couple the dilator hub to the sheath, the locking channel including a guide portion that extends between an opening on a distal end surface of the dilator hub and a locking portion of the locking channel, the locking portion extending in a direction circumferentially around the dilator hub, where the sheath includes a sheath locking sleeve provided at a proximal end of the sheath (e.g., the sheath locking sleeve removably coupled to the sheath hub), the sheath locking sleeve comprising a sleeve body having a proximal end and a distal end and defining a central lumen extending longitudinally between the proximal end and the distal end, a guide disposed on an outer surface of the sleeve body, and where coupling the dilator to the sheath includes: advancing a distal end of the dilator shaft at least partially within the central lumen of the sheath; positioning the dilator hub adjacent a proximal end of the sheath locking sleeve such that the guide projecting from an outer surface of the sheath locking sleeve is received within a locking channel opening on the dilator hub; and rotating the dilator hub in a first direction with respect to the locking sleeve to move the guide along the locking channel into a locked position.

In some implementations, movement of the guide along the locking channel into a locked position includes: movement of the guide along a guide portion of the locking channel toward a locking portion of the locking channel, where the guide portion of the locking channel extends from the distal end of the dilator hub axially towards the proximal end of the dilator hub and the locking portion extends circumferentially around the dilator hub; where further rotation of the dilator hub directs the guide into the locking portion of the locking channel, the locking portion configured to securely engage the guide and fix the axial position of the dilator hub with respect to the sheath locking sleeve.

In some implementations, the locking portion includes a catch that secures the guide within the locking portion of the locking channel, where rotation of the dilator hub in the first direction causes the guide to overcome the bias force of the catch and advance the guide beyond the catch into the locking portion, where the catch secures the guide within the locking portion thereby fixing the axial location of the sheath with respect to the dilator hub.

In some implementations, uncoupling the dilator from the sheath includes: rotating the dilator hub in a second direction with respect to the locking sleeve to slide the guide along the locking channel into an unlocked position; and disengaging the dilator hub from the locking sleeve.

In some implementations, rotating the dilator hub in the second direction causes the guide to side along the locking channel, from the locking portion toward the guide portion, where further rotation of the dilator hub in the second direction directs the guide out of the locking portion of the locking channel and through the guide portion to release the dilator hub from the sheath locking sleeve.

In some implementations, rotation of the dilator hub in the second direction causes the guide to overcome the bias force of the catch and advance from the locking portion to the guide portion of the locking channel.

Another implementation of the present disclosure is directed to a method of inserting a medical device into a blood vessel of a patient, the method comprising: inserting a radially expandable sheath at least partially into the blood vessel of a patient, the sheath including: a continuous inner layer defining a central lumen therethrough, the inner layer having at least one folded portion; and tubular strain relief layer provided over the inner layer positioned at a proximal end of the sheath and extending along at least a portion of a length of the sheath, wherein at least a portion of the strain relief layer is configured to locally expand from an unexpanded configuration at a first diameter to an expanded configuration at a second, larger, diameter in response to an outwardly directed radial force exerted on the lumen by the dilator shaft received within the lumen of the inner layer, and then locally contract at least partially back to the unexpanded configuration as the dilator moves within the lumen. The method further includes coupling a dilator to the sheath, the dilator including: a dilator shaft sized and configured to be received within the central lumen of the expandable sheath; a knob coupled to a proximal end of the dilator shaft; a dilator hub (e.g., coupler) rotatably coupled to the knob; a pin coupling the dilator hub and the dilator shaft and preventing rotational movement therebetween. The method further includes rotating the knob in a first direction causing the dilator shaft to engage the knob and resulting in a corresponding axial movement of the dilator shaft in a distal direction along a longitudinal axis of the dilator thereby increasing a length of the dilator shaft received within the central lumen of the sheath; advancing the dilator shaft through a portion of the central lumen of the sheath corresponding to the strain relief layer such that the dilator shaft exerts an outwardly directed radial force against the central lumen and causes the inner layer and the strain relief layer proximate the dilator shaft to locally expand from an unexpanded configuration to an expanded configuration; rotating the knob in a second direction opposite to the first direction, causing the dilator shaft to engage the knob and resulting in a corresponding axial movement of the dilator shaft in a proximal direction along the longitudinal axis of the dilator thereby reducing a length of the dilator shaft received within the central lumen of the sheath; uncoupling the dilator from the sheath; removing the dilator from the sheath; introducing a medical device into a proximal end of the central lumen of the sheath; advancing the medical device through the sheath; and advancing the medical device beyond a distal opening in the sheath to a treatment site within the blood vessel.

In some implementations, advancing the medical device through the sheath includes: advancing the medical device through the portion of the sheath corresponding to the strain relief layer and thereby exerting an outwardly directed radial force by the medical device against the central lumen (e.g., inner layer) and causing the inner layer and the strain relief layer proximate the medical device to locally expand from an unexpanded configuration to an expanded configuration; locally contracting the strain relief layer towards the unexpanded configuration as the medical device passes through the corresponding portion of the lumen of sheath; advancing the medical device beyond a distal end of the strain relief layer; advancing a medical device through the lumen of the sheath causing the sheath to locally expand from the unexpanded configuration to the expanded configuration at a location proximate the medical device in response to the outwardly directed radial force of the medical device exerted against the inner layer; and locally contracting the sheath at least partially back to the unexpanded configuration as the medical device passes through the lumen.

In some implementations, the medical device is a prosthetic device mounted in a radially crimped state on a delivery apparatus, where advancing the prosthetic device through the lumen of the sheath comprises advancing the delivery apparatus and the prosthetic device through lumen of the sheath and into a vasculature of the patient.

In some implementations, the prosthetic device comprises a prosthetic heart valve and the method further comprises implanting the prosthetic heart valve at a treatment site within the patient.

In some implementations, the prosthetic heart valve is mounted on a balloon catheter of the delivery apparatus as the prosthetic heart valve is advanced through the sheath.

In some implementations, the sheath is inserted into a femoral artery of the patient.

Various aspects of the implementations described above can be combined based on desired sheath system characteristics.

### DESCRIPTION OF DRAWINGS

FIG. 1 is an elevation view of an expandable sheath along with an endovascular delivery apparatus for implanting a prosthetic implant.

FIG. 2 is an elevation view of an expandable sheath including an introducer locking hub, a sheath locking sleeve, and an introducer.

FIG. 3 is an elevation view of the expandable sheath of FIG. 2 along with an endovascular delivery apparatus for implanting a prosthetic implant.

FIG. 4 is an elevation view of an expandable sheath a sheath hub, an introducer locking hub, and a sheath locking sleeve of FIG. 2.

FIG. 5A is a cross sectional view of the sheath hub, introducer locking hub, and sheath locking sleeve of FIG. 2.

FIG. 5B is a cross sectional view of the introducer cap, the sheath hub, the introducer locking hub, the sheath locking sleeve of FIG. 2.

FIG. 6 is a cross sectional view of the introducer cap, sheath hub, introducer locking hub, and sheath locking sleeve of FIG. 2.

FIG. 7 is a distal end view of the sheath locking sleeve of FIG. 2 and the proximal fluid seal of FIGS. 5A-B.

FIG. 8A is a first elevation view of the introducer locking hub of FIG. 2 coupled to an introducer.

FIG. 8B is a second (rotated) elevation view of the introducer locking hub of FIG. 2 coupled to the introducer.

FIG. 8C is a distal end view of the introducer locking hub of FIG. 2 coupled to the introducer.

FIG. 8D is a partial side view of the introducer locking hub of FIG. 2 coupled to the introducer.

FIG. 8E is a partial perspective view of the introducer locking hub of FIG. 2 coupled to the introducer.

FIG. 8F is a partial perspective view of the introducer locking hub of FIG. 2 coupled to the introducer.

FIG. 9A is a distal end view of the introducer locking hub of FIG. 2.

FIG. 9B is a first elevation view of the introducer locking hub of FIG. 2.

FIG. 9C is a proximal end view of the introducer locking hub of FIG. 2.

FIG. 9D is a first perspective view of the introducer locking hub of FIG. 2.

FIG. 9E is a second elevation view of the introducer locking hub of FIG. 2.

FIG. 9F is a second perspective view of the introducer locking hub of FIG. 2.

FIG. 10A is a distal end view of the sheath locking sleeve of FIG. 2.

FIG. 10B is a first elevation view of the sheath locking sleeve of FIG. 2.

FIG. 10C is a proximal end view of the sheath locking sleeve of FIG. 2.

FIG. 10D is a first perspective view of the sheath locking sleeve of FIG. 2.

FIG. 10E is a second elevation view of the sheath locking sleeve of FIG. 2.

FIG. 10F is a second perspective view of the sheath locking sleeve of FIG. 2.

FIG. 11 is a side elevation cross-sectional view of a portion of the expandable sheath of FIGS. 1 and 2.

FIG. 12 is a magnified view of a portion of the expandable sheath of FIGS. 1 and 2.

FIG. 13A is a magnified view of a portion of the expandable sheath of FIGS. 1 and 2 with the outer layer removed for purposes of illustration.

FIG. 13B is a magnified view of a portion of the braided layer of the sheath of FIGS. 1 and 2.

FIG. 14 is a magnified view of a portion of the expandable sheath of FIGS. 1 and 2 illustrating expansion of the sheath as a prosthetic device is advanced through the sheath.

FIG. 15 is a side view of the expandable sheath of FIGS. 1 and 2.

FIG. 16 is a magnified section view of the sheath of FIG. 15 along section line 16-16.

FIG. 17 is cross sectional view of the unexpanded sheath of FIG. 16 along section line 17-17.

FIG. 18 is cross sectional view of the unexpanded sheath of FIG. 15 along section line 18-18.

FIG. 19 is cross sectional view of the unexpanded sheath of FIG. 15 along section line 19-19.

FIG. 20 is cross sectional view of the expanded sheath of FIG. 15 along section line 19-19.

FIG. 21 is a side view of the expandable sheath of FIGS. 1 and 2.

FIG. 22 is a cross section view of the unexpanded sheath of FIG. 21 along section line 22-22.

FIG. 23 is a cross section view of the expanded sheath of FIG. 21 along section line 22-22.

FIG. 24 is a side view of the expandable sheath of FIGS. 1 and 2 and an adjustable length dilator.

FIG. 25 is a first perspective view of the dilator of FIG. 24.

FIG. 26 is a second perspective view of the dilator of FIG. 24.

FIG. 27 is an exploded perspective view of the dilator of FIG. 24.

FIG. 28 is a side view of the dilator of FIG. 24.

FIG. 29 is a cross-sectional view of the dilator along its longitudinal axis with the dilator shaft in a retracted position.

FIG. 30 is a cross-sectional view of the dilator along its longitudinal axis with the dilator shaft in an extended position.

FIG. 31 is a first perspective view of the dilator shaft of FIG. 24.

FIG. 32 is a second perspective view of the dilator shaft of FIG. 24.

FIG. 33 is a first side view of the dilator shaft of FIG. 24.

FIG. 34 is a second side view of the dilator shaft of FIG. 33 rotated 90°.

FIG. 35 is a cross-sectional view of the dilator shaft along its longitudinal axis.

FIG. 36 is a distal end view of the dilator shaft of FIG. 24.

FIG. 37 is a proximal end view of the dilator shaft of FIG. 24.

FIG. 38 is a first perspective view of the knob of FIG. 24.

FIG. 39 is a second perspective view of the knob of FIG. 24.

FIG. 40 is a distal end view of the knob of FIG. 24.

FIG. 41 is a side view of the knob of FIG. 24.

FIG. 42 is a proximal end view of the knob of FIG. 24.

FIG. 43 is a cross-sectional view of the knob of FIG. 24 along its longitudinal axis.

FIG. 44 is a first perspective view of the dilator hub of FIG. 24.

FIG. 45 is a second perspective view of the dilator hub of FIG. 24.

FIG. 46 is a side view of the dilator hub of FIG. 24.

FIG. 47 is a side view of the dilator hub of FIG. 46 rotated 90°.

FIG. 48 is a cross-sectional view of the dilator hub of FIG. 24 along its longitudinal axis.

FIG. 49 is a distal end view of the dilator hub of FIG. 24.

FIG. 50 is a proximal end view of the dilator hub of FIG. 24.

FIG. 51 is a cross-sectional view of the dilator received within an expandable sheath of FIG. 24, in a retracted position.

FIG. 52 is a cross-sectional view of the dilator received within an expandable sheath of FIG. 24, in an extended position.

### DETAILED DESCRIPTION

The following description of certain examples of the inventive concepts should not be used to limit the scope of the claims. Other examples, features, aspects, implementations, and advantages will become apparent to those skilled in the art from the following description. As will be realized, the device and/or methods are capable of other different and obvious aspects, all without departing from the spirit of the inventive concepts. Accordingly, the drawings and descriptions should be regarded as illustrative in nature and not restrictive.

For purposes of this description, certain aspects, advantages, and novel features of the aspects of this disclosure are described herein. The described methods, systems, and apparatus should not be construed as limiting in any way. Instead, the present disclosure is directed toward all novel and nonobvious features and aspects of the various disclosed aspects, alone and in various combinations and sub-combinations with one another. The disclosed methods, systems, and apparatus are not limited to any specific aspect, feature, or combination thereof, nor do the disclosed methods, systems, and apparatus require that any one or more specific advantages be present or problems be solved.

Features, integers, characteristics, compounds, chemical moieties, or groups described in conjunction with a particular aspect or example of the present disclosure are to be understood to be applicable to any other aspect or example described herein unless incompatible therewith. All of the features disclosed in this specification (including any accompanying claims, abstract, and drawings), and/or all of the steps of any method or process so disclosed, may be combined in any combination, except combinations where at least some of such features and/or steps are mutually exclusive. The present disclosure is not restricted to the details of any foregoing aspects. The present disclosure extends to any novel one, or any novel combination, of the features disclosed in this specification (including any accompanying claims, abstract, and drawings), or to any novel one, or any novel combination, of the steps of any method or process so disclosed.

It should be appreciated that any patent, publication, or other disclosure material, in whole or in part, that is said to be incorporated by reference herein is incorporated herein only to the extent that the incorporated material does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

The terms “proximal” and “distal” as used herein refer to regions of a sheath, catheter, or delivery assembly. “Proximal” means that region closest to handle of the device, while “distal” means that region farthest away from the handle of the device.

“Axially” or “axial” as used herein refers to a direction along the longitudinal axis of the sheath.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal aspect. “Such as” is not used in a restrictive sense, but for explanatory purposes.

The expandable introducer sheaths and related componentry described herein can be used to deliver a prosthetic device through a patient's vasculature to a procedure site within the body. The sheath can be constructed to be highly expandable and collapsible in both the radial and axial directions. Disclosed aspects of an expandable sheath can minimize trauma to the vessel by allowing for temporary expansion of a portion of the introducer sheath to accommodate the delivery system, followed by a return to the original diameter once the device passes through.

Expandable introducer sheaths are disclosed in U.S. Pat. No. 8,690,936, entitled “Expandable Sheath for Introducing an Endovascular Delivery Device into a Body,” U.S. Pat. No. 8,790,387, entitled “Expandable Sheath for Introducing an Endovascular Delivery Device into a Body,” U.S. Pat. No. 10,639,152, entitled “Expandable Sheath and Methods of Using the Same,” U.S. Pat. No. 10,792,471, entitled “Expandable Sheath,” U.S. patent application Ser. No. 16/407,057, entitled “Expandable Sheath with Elastomeric Cross Sectional Portions,” U.S. Pat. No. 10,327,896, entitled “Expandable Sheath with Elastomeric Cross Sectional Portions,” U.S. Pat. No. 11,273,062, entitled “Expandable Sheath,” Application No. PCT/US2021/019514, entitled “Expandable sheath for introducing an endovascular delivery device in to a body,” Application No. PCT/US2021/031227, entitled “Expandable sheath for introducing an endovascular delivery device into a body,” Application No. PCT/US2021/031275, entitled “Expandable sheath for introducing an endovascular delivery device into a body,” U.S. application Ser. No. 17/113,268, entitled “Expandable Sheath and Method of Using the Same,” Application No. PCT/US2021/058247, entitled “Self-Expanding, Two Component Sheath,” Application No. PCT/US2022/012785, entitled “Expandable Sheath,” U.S. Pat. No. 11,051,939, entitled “Active Introducer Sheath System,” Application No. PCT/US2022/012684, entitled “Introducer with Sheath Tip Expander,” U.S. application Ser. No. 17/078,556, entitled “Advanced Sheath Patterns,” Application No. PCT/US2021/025038, entitled “Low temperature hydrophilic adhesive for use in expandable sheath for introducing an endovascular delivery device into a body,” Application No. PCT/US2021/050006, entitled “Expandable Sheath Including Reversable Bayonet Locking Hub,” U.S. Provisional Application No. 63/280,251, entitled “Expandable Sheath Gasket to Provide Hemostasis,” the disclosures of which are herein incorporated by reference.

As described herein, disclosed aspects of the introducer sheath prevent the introducer from separating from the sheath during insertion by locking the proximal hub of the introducer to the proximal hub of the sheath. Fixing the introducer and the sheath prevents the introducer from moving backward during insertion, thereby maintaining a snug fit and smooth transition between the introducer and the distal end of the sheath. Furthermore, present aspects can reduce the length of time a procedure takes, as well as reduce the risk of a longitudinal or radial vessel tear, or plaque dislodgement because only one sheath is required, rather than several different sizes of sheaths. Aspects of the present expandable sheath can avoid the need for multiple insertions for the dilation of the vessel.

Disclosed herein are elongate introducer sheaths that are particularly suitable for delivery of implants in the form of implantable heart valves, such as balloon-expandable implantable heart valves. Balloon-expandable implantable heart valves are well-known and will not be described in detail here. An example of such an implantable heart valve is described in U.S. Pat. No. 5,411,552, and also in U.S. Pat. No. 9,393,110, both of which are hereby incorporated by reference. The expandable introducer sheaths disclosed herein may also be used to deliver other types of implantable medical device, such as self-expanding and mechanically expanding implantable heart valves, stents or filters. Beyond transcatheter heart valves, the introducer sheath system can be useful for other types of minimally invasive surgery, such as any surgery requiring introduction of an apparatus into a subject's vessel. For example, the introducer sheath system can be used to introduce other types of delivery apparatus for placing various types of intraluminal devices (e.g., stents, stented grafts, balloon catheters for angioplasty procedures, etc.) into many types of vascular and non-vascular body lumens (e.g., veins, arteries, esophagus, ducts of the biliary tree, intestine, urethra, fallopian tube, other endocrine or exocrine ducts, etc.). The term “implantable” as used herein is broadly defined to mean anything—prosthetic or not—that is delivered to a site within a body. A diagnostic device, for example, may be an implantable.

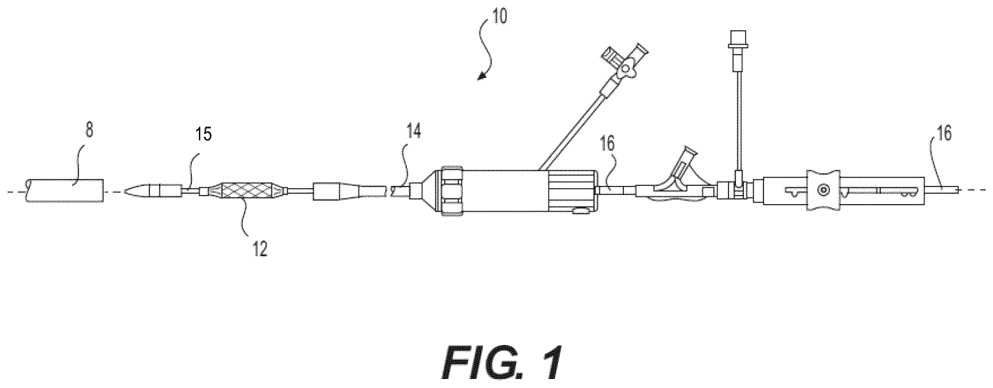

FIG. 1 illustrates an exemplary sheath 8 in use with a representative delivery apparatus 10, for delivering an implant 12, or other type of implantable (e.g., tissue heart valve), to a patient. The delivery apparatus 10 can include a steerable guide catheter 14 (also referred to as a flex catheter) and a balloon catheter 16 extending through the guide catheter 14, and a nose catheter 15 extending through the balloon catheter 16. The guide catheter 14, balloon catheter 16, and nose catheter 15 in the illustrated example are adapted to slide longitudinally relative to each other to facilitate delivery and positioning of the implant 12 at an implantation site in a patient's body as described in detail below. It is contemplated that the sheath 8 can be used with any type of elongated delivery apparatus used for implanting balloon-expandable prosthetic valves, self-expanding prosthetic valves, and other prosthetic devices.

As described in more detail below, in general, the sheath 8 comprises an elongate expandable tube that, in use, is inserted into a vessel (e.g., transfemoral vessel, femoral artery, iliac artery) by passing through the skin of patient, such that the distal end of the sheath 8 is inserted into the vessel. Sheath 8 includes a hemostasis valve and/or sealing features at the proximal end of the sheath, e.g., in the sheath hub 20, that provide hemostasis and prevents blood leakage from the patient through the sheath 8. The sheath 8, including an introducer 6, is advanced into the patient's vasculature. Once positioned the introducer 6 is removed and the delivery apparatus 10 is inserted into/through the sheath 8, and the prosthetic device (implant 12) then be delivered and implanted within patient.

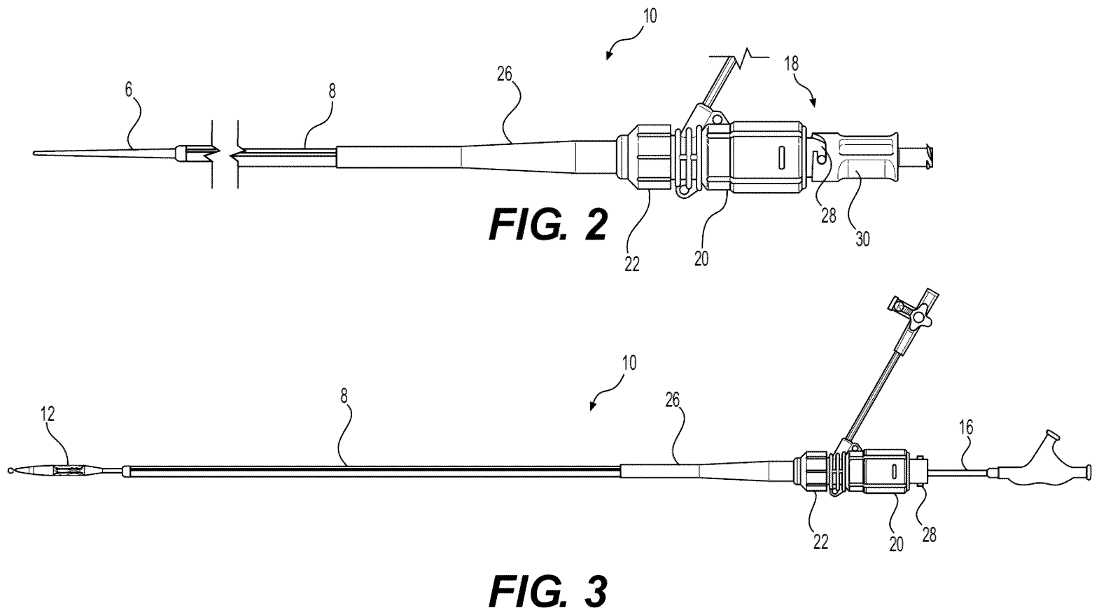

FIGS. 2 and 3, the introducer device/sheath assembly includes a sheath hub 20 at a proximal end of the device and an expandable sheath 8 extending distally from the sheath hub 20. The sheath 8 is coupled to the sheath hub 20 which in turn is removably coupled to a sheath locking system 18. The sheath locking system 18 allows the introducer 6, or other device desired to be removably couped (axially and rotatably) to the sheath 8.

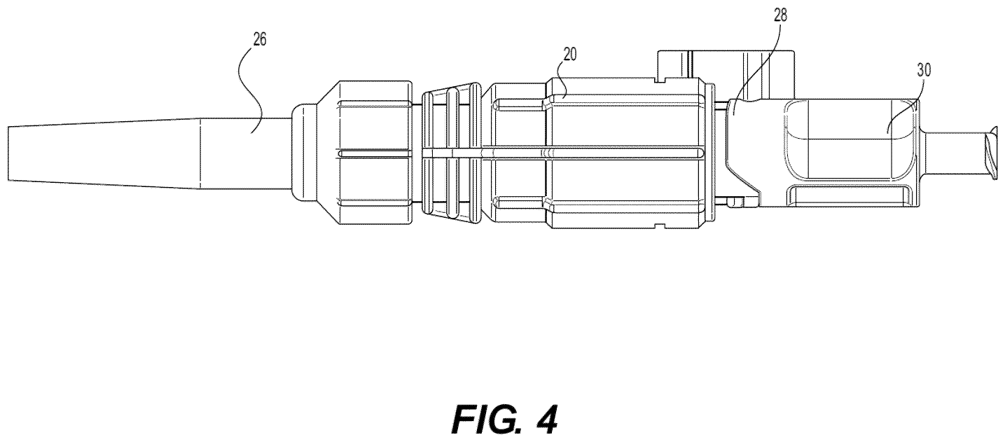

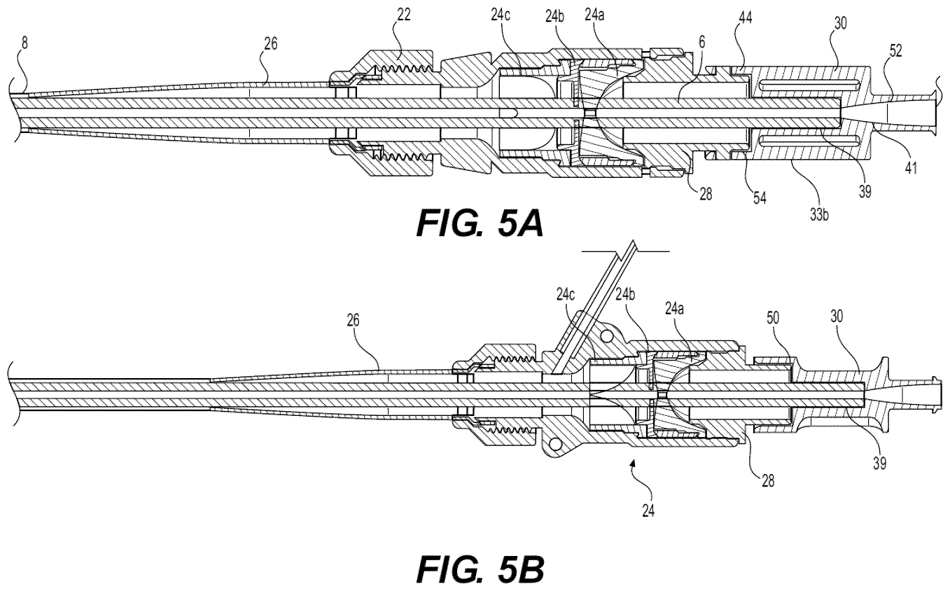

As illustrated in FIGS. 2-6, the sheath hub 20 can function as a handle for the device. sheath hub 20 also provides a housing for necessary seal assemblies and an access point for a secondary lumen (e.g., fluid lumen) in fluid communication with the central lumen of the sheath hub 20. The seal assembly 24, as described above and as shown in FIGS. 5A and 5B, is included in the sheath hub 20. The seal assembly 24 includes a proximal seal 24a, an intermediate seal 24b, and a distal seal 24c. When assembled, the introducer 6 passes through the seal assembly and extends distal of the sheath 8. The proximal seal 24a, the intermediate seal 24b, and the distal seal 24c are each formed to prevent unwanted fluid from advancing in the proximal direction through the sheath hub 20 and proximal of the seal assembly 24. They are each openable and closable to provide pressure variation to affect the desired fluid flow from a physician or technician.

The distal end of the sheath hub 20 includes threads 21 for coupling to a threaded sheath hub cap 22. The sheath 8 is provided between the sheath hub 20 and the sheath hub cap 22 such that coupling the sheath hub cap 22 to the sheath hub 20 fixes the sheath 8 to the sheath hub 20. The sheath hub cap 22 is a cylindrical cap having a cap body having a proximal end and a distal end and defining a central lumen extending longitudinally between the proximal end and the distal end. The sheath hub cap 22 has a larger diameter at its proximal end than at its distal end.

The sheath hub 20 further has receiving slots 48 for coupling the sheath locking system 18, particularly the locking sleeve 28, to the sheath hub 20. The receiving slots 48 are openings which extend around a portion of the diameter of the sheath hub 20 and are sized and configured to accept the interference diameters 66 of the locking sleeve 28. Coupling between the receiving slots 48 and the interference diameters 66 axially and rotationally fixes the locking sleeve 28 and the sheath hub 20 relative to each other.

FIG. 2 illustrates the sheath 8 of FIG. 1 including a sheath locking system 18 which prevents axial and rotational translation of the introducer 6 with respect to the sheath 8. Example locking systems are disclosed in PCT/US2021/050006, entitled “Expandable Sheath Including Reverse Bayonet Locking Hub,” the disclosure of which is incorporated herein by reference. It is contemplated that the locking system disclosed herein can also be used to couple the sheath 8/sheath hub 20 with other delivery system components, catheters, dilators, etc. including the same mating features.

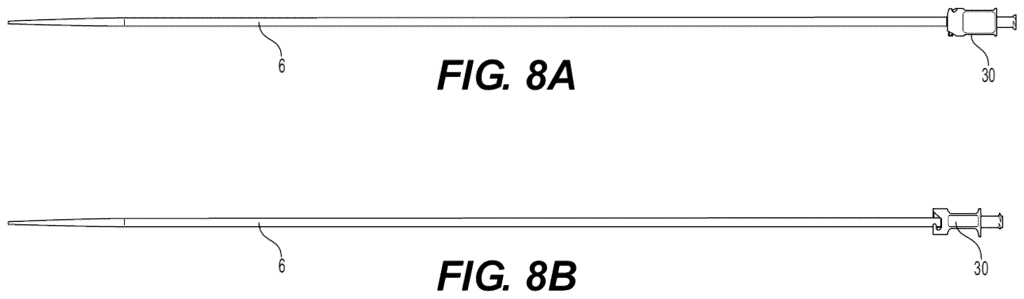

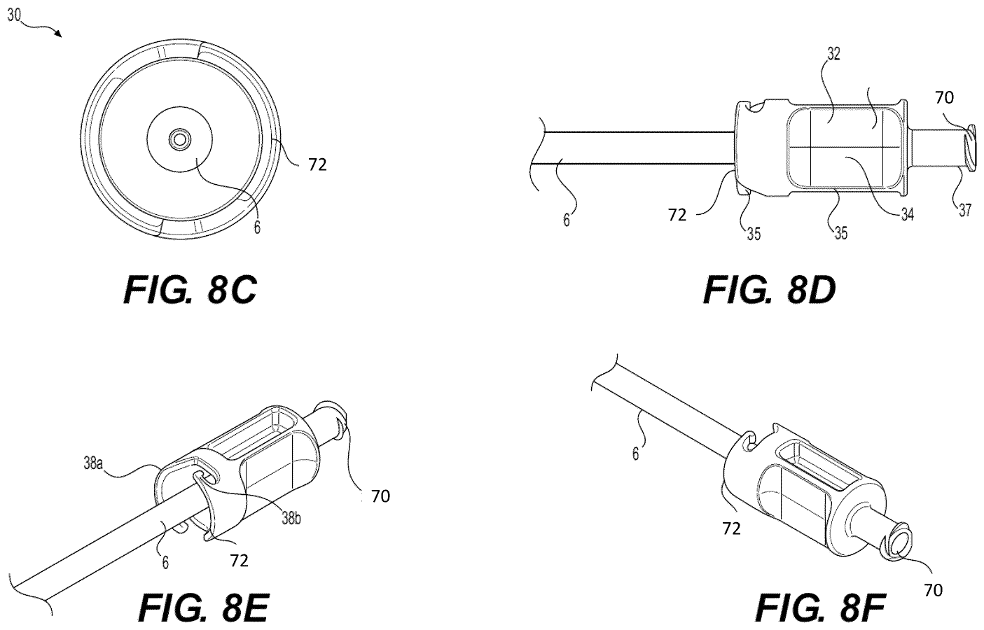

The sheath locking system 18 keeps the introducer 6 fixed with respect to the sheath 8 during insertion without requiring a physician or technician to hold the introducer 6 and the sheath 8 in place at the distal end. As illustrated in FIGS. 8A-8B, the sheath locking system 18 includes a locking sleeve 28 and an introducer locking hub 30 (including corresponding introducer 6). The locking sleeve 28 is coupled to the sheath 8 via the sheath hub 20. The locking sleeve 28 engages the introducer locking hub 30 and is moveable between a locked and unlocked position, thereby fixing the position of the introducer 6 and the sheath 8 and preventing movement therebetween, particularly during insertion into the patient. As will be described in more detail below, the sheath locking system 18 keeps the introducer 6 from separating from the sheath 8 and prevents gaps from forming that can cause patient abrasions and unintended fluid flow between the introducer 6 and the sheath 8 during insertion.

FIGS. 2, 5A-5B and 6, and illustrate the sheath locking sleeve 28 coupled to the introducer locking hub 30 and the sheath hub 20. As will be described in more detail below, the locking sleeve 28 includes a guide 31 that engages a locking channel 38 provided on the introducer locking hub 30. The guide 31 moves within the locking channel 38 between an unlocked position, where the sheath locking sleeve 28 is rotationally and axially movable with respect to the introducer locking hub 30, and a locked position (FIG. 2), where the locking sleeve 28 is axially fixed with respect to the introducer locking hub 30.

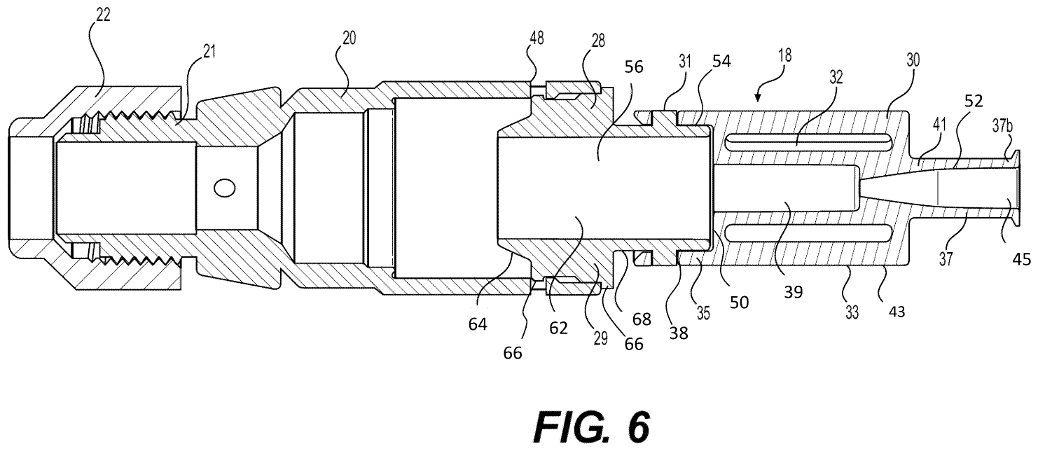

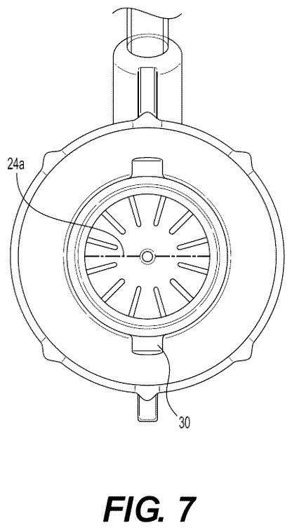

The locking sleeve 28 is illustrated, for example, in FIGS. 10A-10F. The locking sleeve 28 includes an elongated sleeve body 29 with a central lumen 56 extending longitudinally between the proximal end 58 and distal end 60 of the sleeve body 29. As provided in FIG. 6, the central lumen 56 defines a generally cylindrical inner surface 62 of the sheath locking sleeve 28. The central lumen 56 has a diameter of at least 0.3″. In some examples, the diameter ranges from 0.3″ to 0.6″. Preferably, the diameter is about 0.40″. The distal end 60 of the sleeve body 29 also has a frustoconical outer surface 64 that tapers about the distal end 60 to help with positioning the locking sleeve 28 within the sheath hub 20 and abutting the seal assembly 24 (FIGS. 5B and 5B). The locking sleeve 28 also has a plurality of interference diameters 66 that extend radially from the outer surface of the sleeve body 29 around (all or a portion of) the circumference of the locking sleeve 28. As illustrated in FIGS. 5A and 6, the distal interference diameters 66 are sized and configured to engage corresponding recesses and/or slots 48 provided in the sheath hub 20 for securing the locking sleeve 28 to the sheath hub 20, and the distal interference diameter 66 seat against the proximal end of the sheath hub 20.

The locking sleeve 28 includes a guide 31 projecting from the outer surface 68 of the locking sleeve 28. The guide 31 engages a corresponding shaped locking channel 38 in the introducer locking hub 30. The guide 31 extends radially from the outer surface 68 and at least partially around the circumference of the outer surface 68. As provided in FIG. 6, the top surface of the guide 31 does not extend beyond the outer surface of the introducer locking hub 30 when the sheath locking sleeve 28 and the introducer locking hub 30 are coupled. For example, the height of the guide 31 corresponds to the wall thickness of the introducer locking hub 30 proximate the guide when the sheath locking sleeve 28 and the introducer locking hub 30 are coupled. In another example, the top surface of the guide 31 is recessed with respect to the outer surface of the introducer locking hub 30. That is, the height of the guide 31 is less than the wall thickness of the introducer locking hub 30. In other examples, the height of the guide 31 is greater than a wall thickness of the introducer locking hub 30 such that the top surface of the guide 31 extends beyond the outer surface of the introducer locking hub 30 when the sheath locking sleeve 28 and the introducer locking hub 30 are coupled. In some examples, the height/axial length of the guide 31 is between about 0.050″ and about 0.10″. In some examples that height/axial length of the guide 31 is about 0.075″.

As illustrated in FIGS. 10D-10F, the guide 31 is a cylindrically shaped projection. However, it is contemplated that the guide 31 may have any other regular or irregular shape that would facilitate movement of the guide 31 within the locking channel 38 of the introducer locking hub 30. For example, the guide 31 may have an elongated hexagon shape. The guide 31 can have a diameter/width ranging from about 0.05″ to about 0.20″. Preferably the guide 31 has a diameter/width of about 0.100″.

In general, the locking sleeve 28 can be formed from polycarbonate, but in other aspects, the locking sleeve 28 can be formed from rigid plastic, or any other material suitable for providing a strong locking connector for an introducer 6 (metal, composite, etc.).

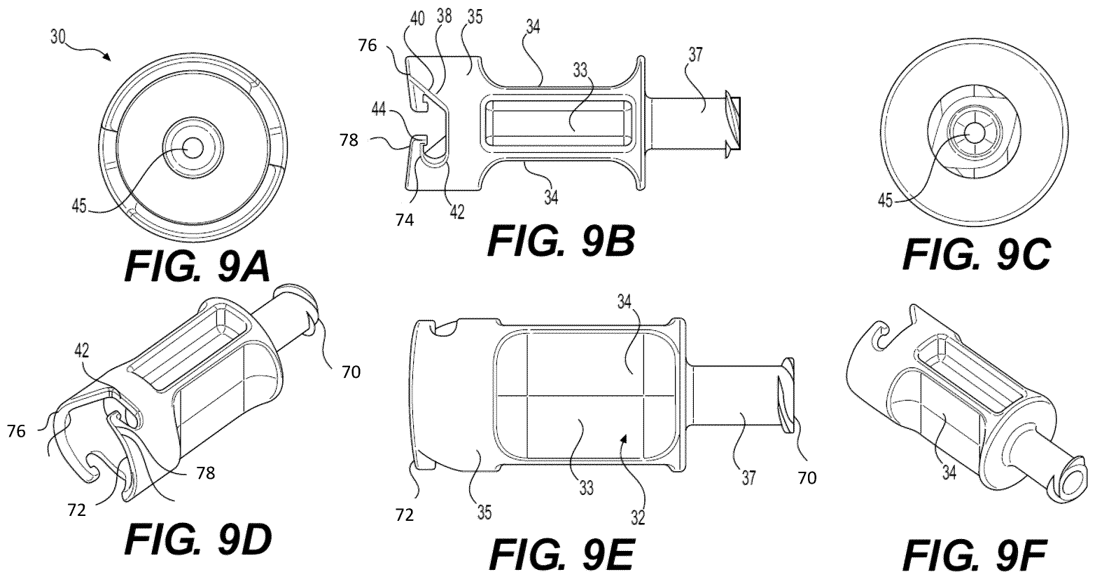

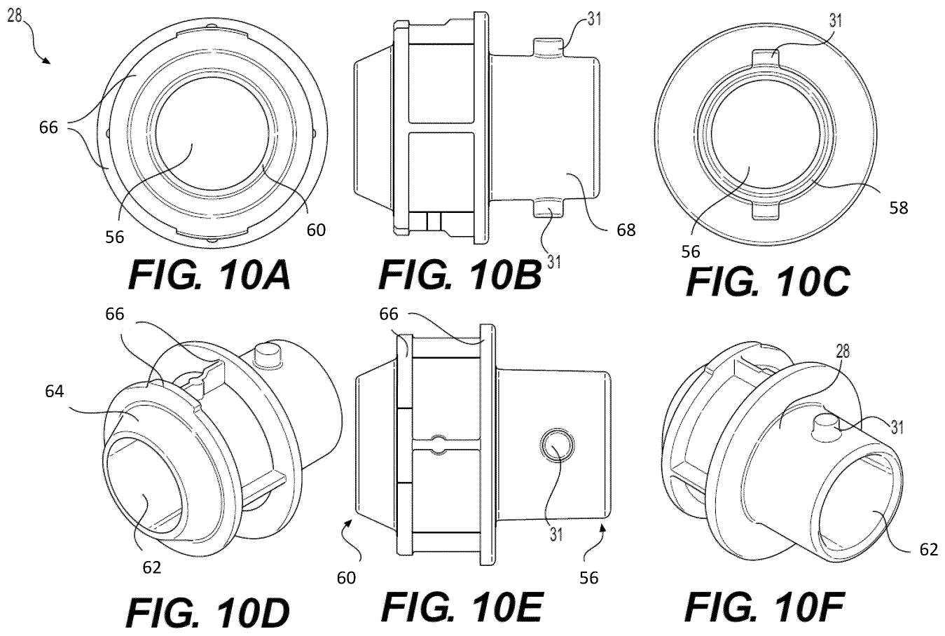

FIGS. 2-6 illustrate the introducer locking hub 30 coupled to the locking sleeve 28. FIGS. 8A-8F show the introducer locking hub 30 coupled to the introducer 6. FIGS. 9A-9F provide multiple view of the introducer locking hub 30. As described herein, the introducer 6 is fixedly coupled to the introducer locking hub 30. The introducer locking hub 30 couples with the locking sleeve 28 to fix the position the introducer 6 (axially and rotationally) with respect to the locking sleeve 28/sheath 8. Each of the introducer 6 and introducer locking hub 30 are described in more detail as follows.

FIGS. 8A-8F illustrate the introducer locking hub 30 with the introducer 6 coupled thereto. Example introducer sheaths are described, for example in U.S. Pat. Nos. 8,690,936 and 8,790,387, the disclosures of which are incorporated herein by reference. As provided in the cross-section views of FIGS. 5A and 5B, the introducer 6 is coupled to the introducer locking hub 30 and extends beyond the distal end of the introducer locking hub 30 body and into the sheath 8. When coupled to the sheath hub 20, the introducer 6 extends through the central lumen 56 of the sheath locking sleeve 28, the sheath hub 20 and the central lumen of the sheath 8. As will be descried below, the sheath 8 generally comprises a radially expandable tubular structure. Passage of the introducer 6 through the sheath 8 and into a patient's vasculature causes the blood vessel to radially expand to about the diameter of the sheath 8. That is, the diameter of the central lumen of the sheath 8 is generally abuts the outer diameter of the introducer 6 such that the introducer 6 provides a mechanism to expand a patient's vessel to accept the sheath.

As provided in FIGS. 8A-8F, the introducer 6 is formed as an elongate body with a central lumen extending therethrough. As shown in FIGS. 5A and 5B, the central lumen of the introducer is aligned with the central lumens of the introducer locking hub 30, the sheath hub 20 and the sheath 8. The introducer 6 is received within a recessed opening 39 provided on an interior surface of the introducer locking hub 30, the recessed opening 39 axially aligned with the central lumen 45 of the introducer locking hub 30. The introducer 6 is coupled to the introducer locking hub 30 at the recessed opening 39. In an example system, the introducer 6 has a diameter corresponding to, or less than, the diameter of the recessed opening 39. In some examples, the introducer 6 is fixedly coupled to the introducer locking hub 30 at the recessed opening 39. For example, the introducer 6 is coupled to the recessed opening 39 of the introducer locking hub 30 by at least one of a press fit, an interference fit, a snap fit, a mechanical fastener, a chemical fastener (e.g., an adhesive), a weld, a thermal process, and/or any other suitable coupling process known in the art.

As described above, the introducer 6 has a central lumen that aligns with the central lumen 45 of the introducer locking hub 30. This joined lumen allows for the passage of surgical equipment and/or medical devices to the treatment site (e.g., a guide wire). In an example system, and as provided in FIGS. 5A and 5B, the central lumen of the introducer 6 has a diameter corresponding to at least a portion of the diameter of the central lumen 45 of the introducer locking hub 30. In general, the corresponding diameter portion is adjacent the distal end of the central lumen 45. In other examples, the diameter of the central lumen 45 at the distal end of the introducer locking hub 30 is slightly larger than the diameter of the central lumen passing through the introducer 6. The central lumen 45 can also define a decreasing tapered portion 41 between the proximal end and the distal end of the introducer locking hub 30 (see FIG. 6). The corresponding diameter portion and decreasing tapered portion 41 allows for smooth transition and delivery of surgical equipment and/or medical device through the introducer locking hub 30 and into the central lumen of the introducer 6.

As illustrated in FIGS. 9A-9F, the introducer locking hub 30 includes a hub body 32 having a proximal end 70 and a distal end 72 and defining a central lumen 45 extending therethrough. The hub body 32 has a first (middle) portion 33, a second (distal) portion 35 which extends distally from the first portion 33 and a third (proximal) portion 37 which extends proximally from the first portion 33. The first portion 33 includes the cylindrically-shaped recessed opening 39 for receiving and retaining the introducer 6 and an outer surface 43. In some examples, the recessed opening 39 has a diameter ranging between 0.15″ and about 0.25″. In some examples, the recessed opening 39 has a diameter ranging between 0.17″ and about 0.20″. In some examples, the recessed opening has a diameter of about 0.194″.

The third (proximal) portion 37 of the introducer locking hub 30 includes the decreasing tapered portion 41 of the central lumen 45. The decreasing tapered portion 41 defining a frustoconical shape with decreasing taper/diameter from the proximal to the distal end of the sheath. It is contemplated that the tapered portion 41 has a minimum diameter of about 0.007″ and a maximum diameter of about 0.194″.

As illustrated in FIGS. 5A and B, when coupled, the central lumen 56 of the locking sleeve 28 is aligned with the central lumen 45 of the introducer locking hub 30. In some examples, the central lumen 56 of the locking sleeve 28 is coaxial with the central lumen 45 of the introducer locking hub 30. When coupled, the proximal end of the locking sleeve 28 is received within the central lumen 45 of the introducer locking hub 30. The proximal end surface of the locking sleeve 28 is adjacent a shoulder 50 provided on an inner surface of the central lumen 45 of the introducer locking hub 30. As illustrated in FIGS. 5A and 5B, the central lumen 45 of the introducer locking hub 30 includes a first portion 52 having a first diameter adjacent the proximal end of the introducer locking hub 30, and a second portion 54 having a second, larger, diameter adjacent the distal end of the introducer locking hub 30. The recessed opening 39 can be considered either a component of the first portion 52 of the central lumen 45, or a separate component of the central lumen 45 located between the first (proximal) portion 52 and the second (distal) portion 54. When the locking sleeve 28 and introducer locking hub 30 are coupled, at least a portion of the sleeve body 29 of the sheath locking sleeve 28 is received within the second portion 54 (larger portion) of the central lumen 45 of the introducer locking hub 30. The central lumen 56 of the sheath locking sleeve 28 is aligned with the central lumen 45 of the introducer locking hub 30 such that they are co-axial and form a smooth inner surface along the combined central lumens of the introducer locking hub 30 and the sheath locking sleeve 28.

As described generally above, the locking sleeve 28 couples to the introducer locking hub 30 via engagement between the guide 31 on the locking sleeve 28 and the locking channel 38 provided in the introducer locking hub 30. As provided in FIGS. 9A-9F, the introducer locking hub 30 includes two locking channels 38. However, it is contemplated that the introducer locking hub 30 can include one locking channel 38 or more than two locking channels 38. The locking channel 38 can be is formed a recess or groove in a surface of the introducer locking hub 30, as a slotted opening, a clip, or as any other feature capable of receiving and securing the guide 31 projecting from the outer surface of the locking sleeve 28 with the introducer locking hub 30. Illustrated in FIG. 9B, the locking channels 38 provide an interface to secure the sheath locking sleeve 28 to the introducer locking hub 30 and ensure a fixed axial position between the introducer 6 and the sheath 8.

The locking channel 38 is formed on the distal end of the introducer locking hub 30. The locking channel 38 includes an opening on the distal end surface that leads to an angled guide portion 40 that transitions to a locking portion 42. The guide portion 40 is configured to direct the guide 31 of the locking sleeve 28 in an axial and circumferential direction along the side wall of the guide portion 40 towards the locking portion 42 upon rotation of the introducer locking hub 30 and/or the sheath locking sleeve 28. The locking portion 42 is configured to securely engage the guide 31, fixing the axial position of the introducer locking hub 30 with respect to the sheath locking sleeve 28. As illustrated in FIG. 9B, the guide portion 40 of the locking channel 38 extends from the distal end of the introducer locking hub 30 axially towards the proximal end of the introducer locking hub 30 and circumferentially around the introducer locking hub 30. For example, the guide portion 40 of the locking channel 38 can be described as extending helically around/along a length of the introducer locking hub 30 or on an angle from the distal end of the introducer locking hub 30.

As illustrated in FIGS. 9B and 9D, the locking portion 42 of the locking channel 38 extends at an angle from the end of the guide portion 40. As provided in FIG. 9B, the angle between the centerline of the guide portion 40 and the centerline of the locking portion 42 is greater than 90-degrees. In another example, the angle between the centerline of the guide portion 40 and the centerline of the locking portion 42 is about 120-degrees. In an example system, the locking portion 42 extends around a portion of the circumference of the introducer locking hub 30. The locking portion 42 can extend parallel to the distal end of the introducer locking hub 30. In an example system, the length of the guide portion 40 (measured along its centerline) is greater than a length of the locking portion 42 (measured along its centerline). In another example, the length of the guide portion 40 equals or is less than a length of the locking portion 42.

The locking portion 42 can include a catch 44 for securing the guide 31 within the locking portion 42 of the locking channel 38 and forming a partial barrier for the guide 31 within the locking portion 42. As illustrated in FIG. 9B, the catch 44 includes a projection that extends from a side wall 74 of the locking portion 42 and releasably secures the guide 31 within the locking channel 38. The catch 44 extends from the side wall 42a of the locking portion 42 in a proximal direction towards the center line of the locking portion 42 and has a height sufficient to retain the guide 31 between the catch 44 and the end of the locking portion 42.

The distal end 72 of the introducer locking hub 30 can include features for biasing the guide 31 towards the locking channel 38. For example, the distal end of the introducer locking hub 30 can include a tapered surface angled toward an opening of the locking channel 38. As illustrated in FIG. 9B, the distal end 72 of the introducer locking hub 30 includes a first tapered surface 76 (angled towards a leading edge of the opening of the locking channel 38 and a second tapered surface 78 angled towards the trailing edge of the opening of the locking channel 38.

In use, engagement between the guide 31 and the guide portion 40 of the locking channel 38 is configured to bias the locking sleeve 28 in a proximal axial direction toward the proximal end 70 of the introducer locking hub 30 (towards a locked position) when the sheath locking sleeve 28 is rotated in a first axial direction. In this direction the guide 31 advances toward the locking portion 42 of the locking channel 38 into the locked position. Alternatively, engagement between the guide 31 and the locking portion 42 of the locking channel 38 is configured to bias the locking sleeve 28 in a distal axial direction toward the distal end of the introducer locking hub 30 (towards an unlocked position) when the sheath locking sleeve 28 is rotated in a second (opposite) axial direction. In the second direction, the guide 31 advances away from the locking portion 42 of the locking channel 38, to the unlocked position. When the guide 31 is in the locked position and retained with by locking portion 42 by catch 44, rotation in the second direction causes the guide 31 to bias against the catch 44 overcoming the oppositional forces of the catch 44, and moving the guide 31 from the locked to the unlocked position.

As illustrated in FIGS. 8A-9F, the outer surface of the introducer locking hub body 32 includes gripping features and/or surfaces for a physician or technician to use when manipulating the introducer locking hub 30. As provided in FIG. 9B, the introducer locking hub body 32 can include a two recessed gripping surfaces 34 on opposite sides of the longitudinal axis of the introducer locking hub 30. When the introducer locking hub 30 is viewed from the side, the gripping surfaces 34 define a dog-bone/barbell shape to the hub body 32, i.e., a shape having a smaller diameter/width center portion and larger diameter/width end portions. In an example system, the gripping surfaces 34 are provided along at least 40% of the length of the introducer locking hub body 32. In another example, the gripping surfaces 34 are provided along at least 50% of the length of the introducer locking hub body 32.

In general, the introducer locking hub 30 can be formed from polycarbonate, but in other aspects the introducer locking hub 30 can be formed from rigid plastic, or any other material suitable for providing a locking mechanism for an introducer 6 (metal, composite, etc.).

As described above, the introducer device/sheath assembly includes an expandable sheath 8 extending distally from the sheath hub 20. The expandable sheath 8 has a central lumen to guide passage of the delivery apparatus 10 for the medical device/prosthetic heart valve. In an alternative aspect, the introducer device/sheath assembly need not include the sheath hub 20. For example, the sheath 8 can be an integral part of a component of the sheath assembly, such as the guide catheter. As described above, the sheath 8 can have a natural, unexpanded outer diameter that will expand locally upon passage of the medical device.

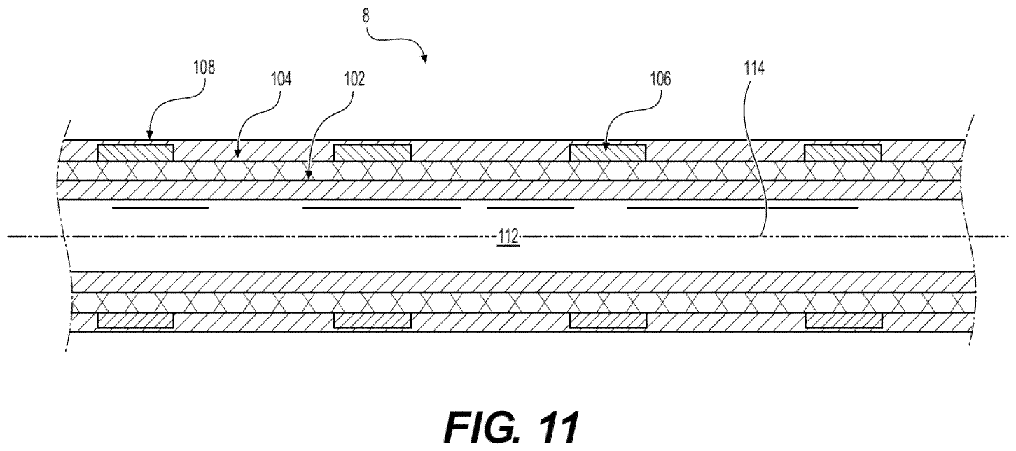

In some aspects, the expandable sheath 8 can comprise a plurality of coaxial layers extending along at least a portion of the length of the sheath 8. The structure of the coaxial layers is described in more detail below with respect to FIGS. 11-23. Example expandable sheaths including coaxial layers are described, for example, in U.S. patent application Ser. No. 16/378,417, entitled “Expandable Sheath,” and U.S. patent application Ser. No. 17/716,882, entitled “Expandable Sheath,” the disclosures of which are herein incorporated by reference.

Various aspects of the coaxial layered structure of the sheath 8 are described herein. For example, in reference to the example sheath 8 illustrated in FIGS. 11-14, the expandable sheath 8 can include a number of layers including an inner layer 102 (also referred to as an inner layer), a second layer 104 disposed around and radially outward of the inner layer 102, a third layer 106 disposed around and radially outward of the second layer 104, and a fourth outer layer 108 (also referred to as an outer layer) disposed around and radially outward of the third layer 106. In the illustrated configuration, the inner layer 102 can define the lumen 112 of the sheath extending along a central axis 114 through which the delivery apparatus travels into the patient's vessel in order to deliver, remove, repair, and/or replace a prosthetic device, moving in a direction along the longitudinal axis of the sheath 8.

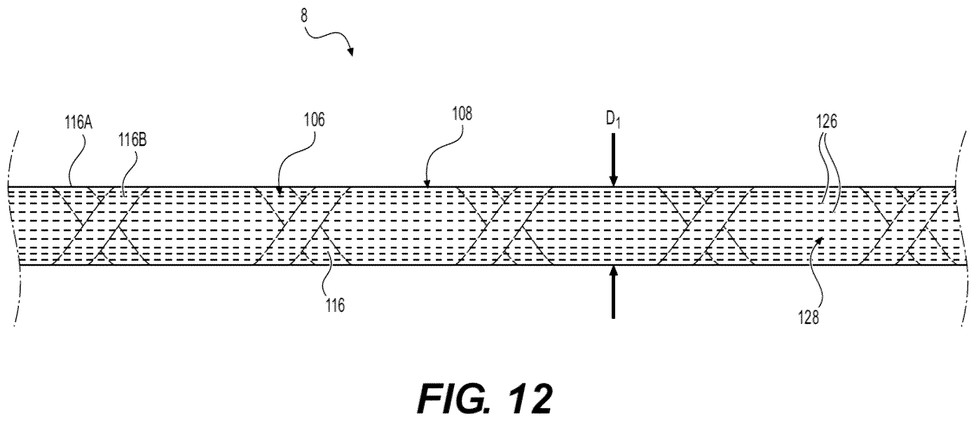

Referring to FIG. 12, when the sheath 8 is in an unexpanded state, various layers of the sheath, e.g., the inner layer 102 and/or the outer layer 108, can form longitudinally-extending folds or creases such that the surface of the sheath comprises a plurality of ridges 126 (also referred to herein as “folds”). The ridges 126 can be circumferentially spaced apart from each other by longitudinally-extending valleys 128. When the sheath expands beyond its natural diameter D₁, the ridges 126 and the valleys 128 can level out or be taken up as the surface radially expands and the circumference increases, as further described below. When the sheath 8 collapses back to its natural diameter, the ridges 126 and valleys 128 can reform.

In some aspects, the inner layer 102 and/or the outer layer 108 can comprise a relatively thin layer of polymeric material. For example, in some aspects the thickness of the inner layer 102 can be from 0.01 mm to 0.5 mm, 0.02 mm to 0.4 mm, or 0.03 mm to 0.25 mm. In some aspects, the thickness of the outer layer 108 can be from 0.01 mm to 0.5 mm, 0.02 mm to 0.4 mm, or 0.03 mm to 0.25 mm.

In some examples, the inner layer 102 and/or the outer layer 108 can comprise a lubricious, low-friction, and/or relatively non-elastic material. In particular aspects, the inner layer 102 and/or the outer layer 108 can comprise a polymeric material having a modulus of elasticity of 400 MPa or greater. Exemplary materials can include ultra-high-molecular-weight polyethylene (UHMWPE) (e.g., Dyneema®), high-molecular-weight polyethylene (HMWPE), or polyether ether ketone (PEEK). With regard to the inner layer 102 in particular, such low coefficient of friction materials can facilitate passage of the prosthetic device through the lumen 112. Other suitable materials for the inner and outer layers can include polyimide, polytetrafluoroethylene (PTFE), expanded polytetrafluoroethylene (ePTFE), ethylene tetrafluoroethylene (ETFE), nylon, polyethylene, polyamide, polyether block amide (e.g., Pebax), and/or combinations of any of the above. Some aspects the sheath 8 can include a lubricious liner on the inner surface of the inner layer 102. Examples of suitable lubricious liners include materials that can further reduce the coefficient of friction of the inner layer 102, such as PTFE, polyethylene, polyvinylidine fluoride, and combinations thereof. Suitable materials for a lubricious liner also include other materials desirably having a coefficient of friction of 0.1 or less.

Additionally, some aspects of the sheath 8 can include an exterior hydrophilic coating on the outer surface of the outer layer 108. Such a hydrophilic coating can facilitate insertion of the sheath 8 into a patient's vessel, reducing potential damage. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N.V, Heerlen, the Netherlands), as well as other hydrophilic coatings (e.g., PTFE, polyethylene, polyvinylidine fluoride), are also suitable for use with the sheath 8. Such hydrophilic coatings may also be included on the inner surface of the inner layer 102 to reduce friction between the sheath and the delivery system, thereby facilitating use and improving safety. In some aspects, a hydrophobic coating, such as Perylene, may be used on the outer surface of the outer layer 108 or the inner surface of the inner layer 102 in order to reduce friction.

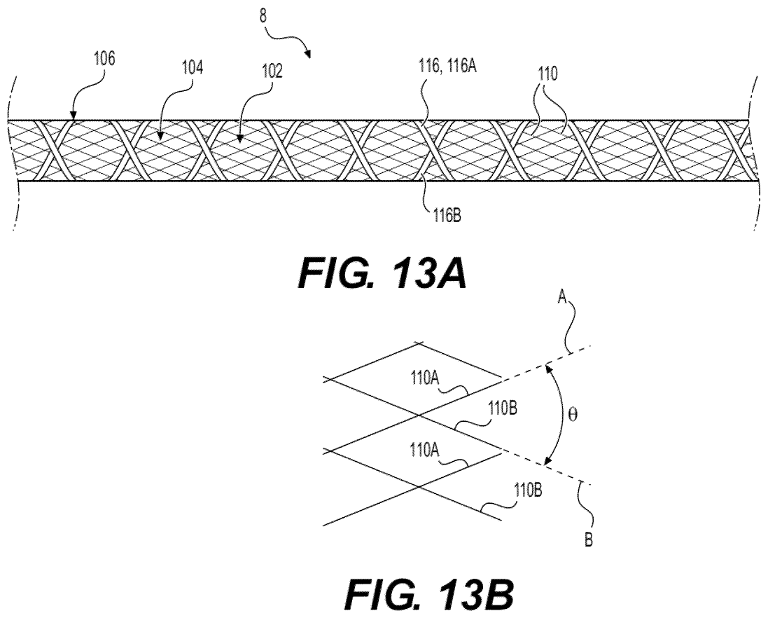

In some aspects, the second layer 104 can be a braided layer. FIGS. 13A and 13B illustrate the sheath 8 with the outer layer 108 removed to expose the elastic third layer 106. With reference to FIGS. 13A and 13B, the braided second layer 104 can comprise a plurality of members or filaments 110 (e.g., metallic or synthetic wires or fibers) braided together. The braided second layer 104 can have any desired number of filaments 110, which can be oriented and braided together along any suitable number of axes. For example, with reference to FIG. 13B, the filaments 110 can include a first set of filaments 110A oriented parallel to a first axis A, and a second set of filaments 110B oriented parallel to a second axis B. The filaments 110A and 110B can be braided together in a biaxial braid such that filaments 110A oriented along axis A form an angle θ with the filaments 110B oriented along axis B. In some aspects, the angle θ can be from 5° to 70°, 10° to 60°, 10° to 50°, or 10° to 45°. In the illustrated example, the angle θ is 45°. In other aspects, the filaments 110 can also be oriented along three axes and braided in a triaxial braid, or oriented along any number of axes and braided in any suitable braid pattern. The braided second layer 104 can extend along substantially the entire length L of the sheath 8, or alternatively, can extend only along a portion of the length of the sheath. In particular aspects, the filaments 110 can be wires made from metal (e.g., Nitinol, stainless steel, etc.), or any of various polymers or polymer composite materials, such as carbon fiber. In some aspects, the filaments 110 can be round, and can have a diameter of from 0.01 mm to 0.5 mm, 0.03 mm to 0.4 mm, or 0.05 mm to 0.25 mm. In other aspects, the filaments 110 can have a flat cross-section with dimensions of 0.01 mm×0.01 mm to 0.5 mm×0.5 mm, or 0.05 mm×0.05 mm to 0.25 mm×0.25 mm. In one aspect, filaments 110 having a flat cross-section can have dimensions of 0.1 mm×0.2 mm. However, other geometries and sizes are also suitable for some aspects. If braided wire is used, the braid density can be varied. Some aspects have a braid density of from ten picks per inch to eighty picks per inch, and can include eight wires, sixteen wires, or up to fifty-two wires in various braid patterns. In other aspects, the second layer 104 can be laser cut from a tube, or laser-cut, stamped, punched, etc., from sheet stock and rolled into a tubular configuration. The second layer 104 can also be woven or knitted, as desired.

The third layer 106 can be a resilient, elastic layer (also referred to as an elastic material layer). In some aspects, the elastic third layer 106 can be configured to apply radially inward force to the underlying layers 102 and 104 in a radial direction (e.g., toward the central axis 114 of the sheath) when the sheath expands beyond its natural diameter by passage of the delivery apparatus through the sheath. Stated differently, the elastic third layer 106 can be configured to apply encircling/radially inward pressure to the layers of the sheath beneath the elastic third layer 106 to counteract expansion of the sheath. The radially inwardly directed force is sufficient to cause the sheath to collapse radially back to its unexpanded state after the delivery apparatus is passed through the sheath.

In the illustrated example, the elastic third layer 106 can comprise one or more members configured as strands, ribbons, or bands 116 helically wrapped around the braided second layer 104. For example, in the illustrated aspect the elastic third layer 106 comprises two elastic bands 116A and 116B wrapped around the braided second layer 104 with opposite helicity, although the elastic layer may comprise any number of bands depending upon the desired characteristics. The elastic bands 116A and 116B can be made from, for example, any of a variety of natural or synthetic elastomers, including silicone rubber, natural rubber, any of various thermoplastic elastomers, polyurethanes such as polyurethane siloxane copolymers, urethane, plasticized polyvinyl chloride (PVC), styrenic block copolymers, polyolefin elastomers, etc. In some aspects, the elastic layer can comprise an elastomeric material having a modulus of elasticity of 200 MPa or less. In some aspects, the elastic third layer 106 can comprise a material exhibiting an elongation to break of 200% or greater, or an elongation to break of 400% or greater. The elastic third layer 106 can also take other forms, such as a tubular layer comprising an elastomeric material, a mesh, a shrinkable polymer layer such as a heat-shrink tubing layer, etc. In lieu of, or in addition to, the elastic third layer 106, the sheath 8 may also include an elastomeric or heat-shrink tubing layer around the outer layer 108. Examples of such elastomeric layers are disclosed in U.S. Publication No. 2014/0379067, U.S. Publication No. 2016/0296730, and U.S. Publication No. 2018/0008407, which are incorporated herein by reference. In other aspects, the elastic third layer 106 can also be radially outward of the polymeric outer layer 108.

In some aspects, one or both of the inner layer 102 and/or the outer layer 108 can be configured to resist axial elongation of the sheath 8 when the sheath expands. More particularly, one or both of the inner layer 102 and/or the outer layer 108 can resist stretching against longitudinal forces caused by friction between a prosthetic device and the inner surface of the sheath 8 such that the length L remains substantially constant as the sheath expands and contracts. As used herein with reference to the length L of the sheath, the term “substantially constant” means that the length L of the sheath increases by not more than 1%, by not more than 5%, by not more than 10%, by not more than 15%, or by not more than 20%. Meanwhile, with reference to FIG. 13B, the filaments 110A and 110B of the braided second layer 104 can be allowed to move angularly relative to each other such that the angle θ changes as the sheath expands and contracts. This, in combination with the longitudinal folds/ridges 126 in the layers 102 and 108, can allow the lumen 112 of the sheath to expand as a prosthetic device is advanced through it.

For example, in some aspects the inner layer 102 and the outer layer 108 can be heat-bonded during the manufacturing process such that the braided second layer 104 and the elastic third layer 106 are encapsulated between the layers 102 and 108. More specifically, in some aspects the inner layer 102 and the outer layer 108 can be adhered to each other through the spaces between the filaments 110 of the braided second layer 104 and/or the spaces between the elastic bands 116. The layers 102 and 108 can also be bonded or adhered together at the proximal and/or distal ends of the sheath. In some aspects, the layers 102 and 108 are not adhered to the filaments 110. This can allow the filaments 110 to move angularly relative to each other, and relative to the layers 102 and 108, allowing the diameter of the braided second layer 104, and thereby the diameter of the sheath, to increase or decrease. As the angle θ between the filaments 110A and 110B changes, the length of the braided second layer 104 can also change. For example, as the angle θ increases, the braided second layer 104 can foreshorten, and as the angle θ decreases, the braided second layer 104 can lengthen to the extent permitted by the areas where the layers 102 and 108 are bonded. However, because the braided second layer 104 is not adhered to the layers 102 and 108, the change in length of the braided layer that accompanies a change in the angle θ between the filaments 110A and 110B does not result in a significant change in the length L of the sheath.

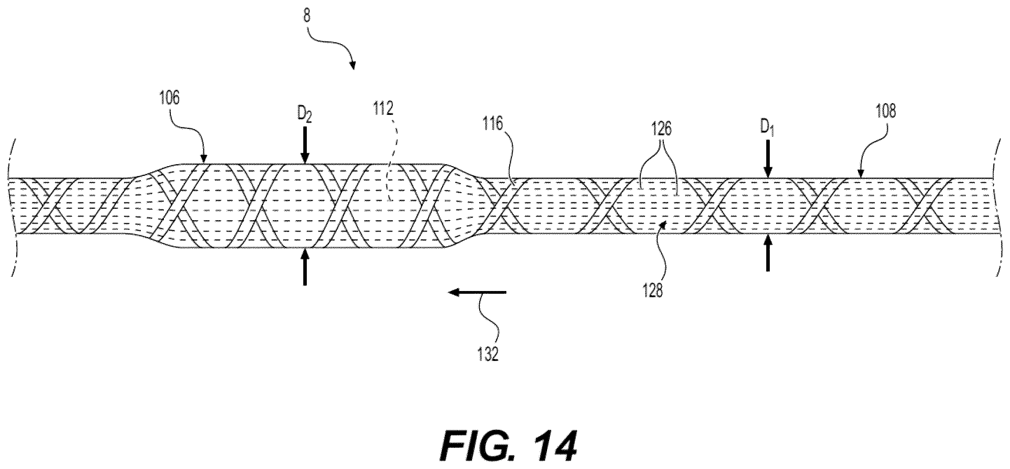

FIG. 14 illustrates radial expansion of the sheath 8 as a prosthetic device (e.g., implant 12) is passed through the sheath 8 in the direction of arrow 132 (e.g., distally). As the prosthetic device (implant 12) is advanced through the sheath 8, the sheath can resiliently expand to a second diameter D₂ that corresponds to a size or diameter of the prosthetic device. As the prosthetic device (implant 12) is advanced through the sheath 8, the prosthetic device can apply longitudinal force to the sheath in the direction of motion by virtue of the frictional contact between the prosthetic device and the inner surface of the sheath. However, as noted above, the inner layer 102 and/or the outer layer 108 can resist axial elongation such that the length L of the sheath remains constant, or substantially constant. This can reduce or prevent the braided second layer 104 from lengthening, and thereby constricting the lumen 112.

Meanwhile, the angle θ between the filaments 110A and 110B can increase as the sheath expands to the second diameter D₂ to accommodate the prosthetic valve. This can cause the braided second layer 104 to foreshorten. However, because the filaments 110 are not engaged or adhered to the layers 102 or 108, the shortening of the braided second layer 104 attendant to an increase in the angle θ does not affect the overall length L of the sheath. Moreover, because of the longitudinally-extending folds/ridges 126 formed in the layers 102 and 108, the layers 102 and 108 can expand to the second diameter D₂ without rupturing, in spite of being relatively thin and relatively non-elastic. In this manner, the sheath 8 can resiliently expand from its natural diameter D₁ to a second diameter D₂ that is larger than the diameter D₁ as a prosthetic device is advanced through the sheath, without lengthening, and without constricting. Thus, the force required to push the prosthetic implant through the sheath is significantly reduced.

Additionally, because of the radial force applied by the elastic third layer 106, the radial expansion of the sheath 8 can be localized to the specific portion of the sheath occupied by the prosthetic device. For example, with reference to FIG. 14, as the prosthetic device (implant 12) moves distally through the sheath 8, the portion of the sheath immediately proximal to the prosthetic device (e.g., implant 12) can radially collapse back to the initial diameter D₁ under the influence of the elastic third layer 106. The layers 102 and 108 can also buckle as the circumference of the sheath is reduced, causing the ridges 126 and the valleys 128 to reform. This can reduce the size of the sheath required to introduce a prosthetic device of a given size. Additionally, the temporary, localized nature of the expansion can reduce trauma to the blood vessel into which the sheath is inserted, along with the surrounding tissue, because only the portion of the sheath occupied by the prosthetic device expands beyond the sheath's natural diameter and the sheath collapses back to the initial diameter once the device has passed. This limits the amount of tissue that must be stretched in order to introduce the prosthetic device, and the amount of time for which a given portion of the vessel must be dilated.

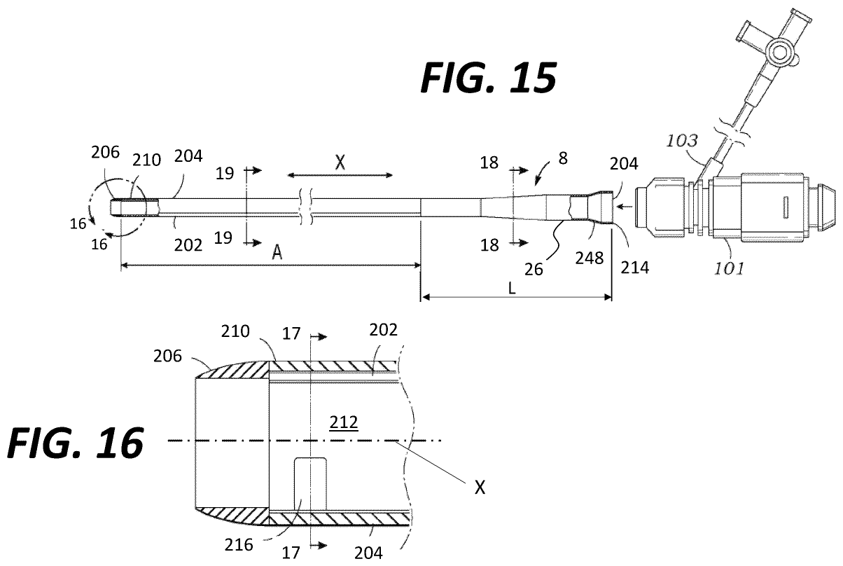

FIGS. 15-23 illustrate various features of the coaxial layered structure of the expandable sheath 8 of FIG. 1 according to another aspect. Similar reference numbers are used to describe like elements. It is to be understood that the variations (e.g., materials and alternate configurations) described above with reference to FIGS. 11-14 can also apply to the example shown in FIGS. 15-23. Furthermore, the variations described below with reference to FIGS. 15-23 can also be applied to the sheath described in FIGS. 11-14.

Similar to various aspects of the sheath 8 described above in reference to FIGS. 11-14, the sheath 8 of FIGS. 15-23 includes a plurality of layers. For example, the sheath 8 illustrated in FIGS. 15-23, also includes an inner layer 202 and an outer layer 204 disposed around the inner layer 202. The inner layer 202 can define a lumen 212 through which the delivery apparatus travels into the patient's vessel in order to deliver, remove, repair, and/or replace a prosthetic device, moving in a direction along the longitudinal axis X. Similar to the sheath illustrated in FIGS. 11-14, as the prosthetic device passes through the sheath 8, the sheath 8 locally expands from a first, resting/unexpanded diameter to a second, expanded diameter to accommodate the prosthetic device. After the prosthetic device passes through a particular location of the sheath 8, each successive expanded portion or segment of the sheath 8 at least partially returns to the smaller, resting/unexpanded diameter. In this manner, the sheath 8 can be considered self-expanding, in that it does not require use of a balloon, dilator, and/or obturator to expand.

Similar to the examples above, the inner and outer layers 202, 204 can comprise any suitable materials. Suitable materials for the inner layer 202 include polytetrafluoroethylene (PTFE), ethylene tetrafluoroethylene (ETFE), nylon, polyethylene, polyether block amide (e.g., Pebax), and/or combinations thereof. In one specific implementation the inner layer 202 can comprise a lubricious, low-friction, or hydrophilic material, such as PTFE. Such low coefficient of friction materials can facilitate passage of the prosthetic device through the lumen defined by the inner layer 202. In some examples, the inner layer 202 can have a coefficient of friction of less than about 0.1. Some examples of the sheath 8 can include a lubricious liner on the inner surface of the inner layer 202. Examples of suitable lubricious liners include materials that can further reduce the coefficient of friction of the inner layer 202, such as PTFE, polyethylene, polyvinylidene fluoride, and combinations thereof. Suitable materials for a lubricious liner also include other materials desirably having a coefficient of friction of about 0.1 or less.

Suitable materials for the outer layer 204 include nylon, polyethylene, Pebax, HDPE, polyurethanes (e.g., Tecoflex), and other medical grade materials. In one implementation, the outer layer 204 can comprise high density polyethylene (HDPE) and Tecoflex (or other polyurethane material) extruded as a composite. In some implementations, the Tecoflex can act as an adhesive between the inner layer 202 and the outer layer 204 and may only be present along a portion of the inner surface of the outer layer 204. Other suitable materials for the inner and outer layers are also disclosed in U.S. Pat. Nos. 8,690,936 and 8,790,387, which are incorporated herein by reference.

Additionally, some examples of the sheath 8 include an exterior hydrophilic coating on the outer surface of the outer layer 204. Such a hydrophilic coating can facilitate insertion of the sheath 100 into a patient's vessel. Examples of suitable hydrophilic coatings include the Harmony™ Advanced Lubricity Coatings and other Advanced Hydrophilic Coatings available from SurModics, Inc., Eden Prairie, MN. DSM medical coatings (available from Koninklijke DSM N.V, Heerlen, the Netherlands), as well as other hydrophilic coatings (e.g., PTFE, polyethylene, polyvinylidene fluoride), are also suitable for use with the sheath 100.

FIG. 16 provides a partial cross-section of the distal end of the sheath 8 along section line 16-16 identified in FIG. 15. As described herein, the sheath 8 can be inserted into a vessel (e.g., the femoral or iliac arteries) by passing through the skin of patient, such that a soft tip portion 206 at the distal end 210 of the sheath 8 is inserted into the vessel. As best seen in FIG. 16, the soft tip portion 206 can comprise, in some examples, low density polyethylene (LDPE) and can be configured to minimize trauma or damage to the patient's vessels as the sheath is navigated through the vasculature. For example, the soft tip portion 206 can be slightly tapered to facilitate passage through the vessels. The soft tip portion 206 can be secured to the distal end 210 of the sheath 8, such as by thermally bonding the soft tip portion 206 to the inner and outer layers of the sheath 8. Such a soft tip portion 206 can be provided with a lower hardness than the other portions of the sheath 8. In some examples, the soft tip portion 206 can have a Shore hardness from about 25 D to about 40 D. The tip portion 206 is configured to be radially expandable to allow a prosthetic device to pass through the distal opening of the sheath 208. For example, the tip portion 206 can be formed with a weakened portion, such as an axially extending score line or perforated line that is configured to split and allow the tip portion 206 to expand radially when the prosthetic device passes therethrough.

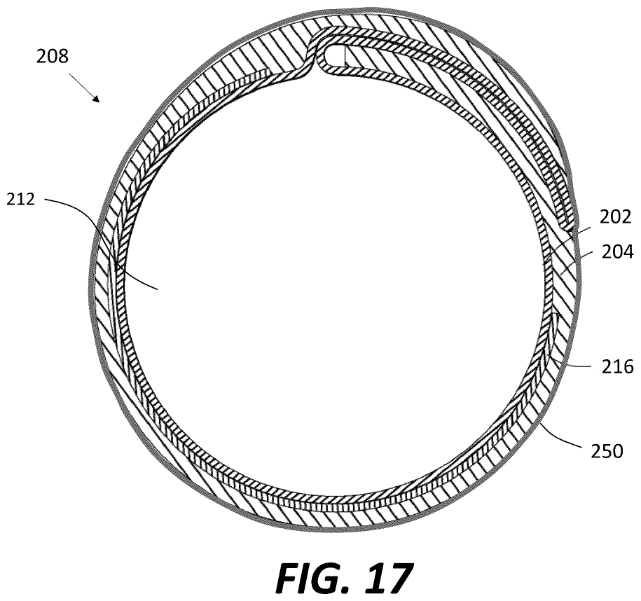

FIG. 17 shows a cross-section view of the sheath 8 taken near the distal end 210 of the sheath 8 as indicated by section line 17-17 in FIG. 16. As illustrated in FIGS. 16 and 17, the sheath 8 can include at least one radiopaque filler or marker, such as a discontinuous, or C-shaped, band (marker 216) positioned near the distal end 210 of the sheath 8. The marker 216 can be associated with the inner and/or outer layers 202, 204 of the sheath 8. For example, as shown in FIG. 17, the marker 216 can be positioned between the inner layer 202 and the outer layer 204. In alternative examples, the marker 216 can be associated with the outer surface of the outer layer 204. In some examples, the marker 216 can be embedded or blended within the inner or outer layers 202, 204.

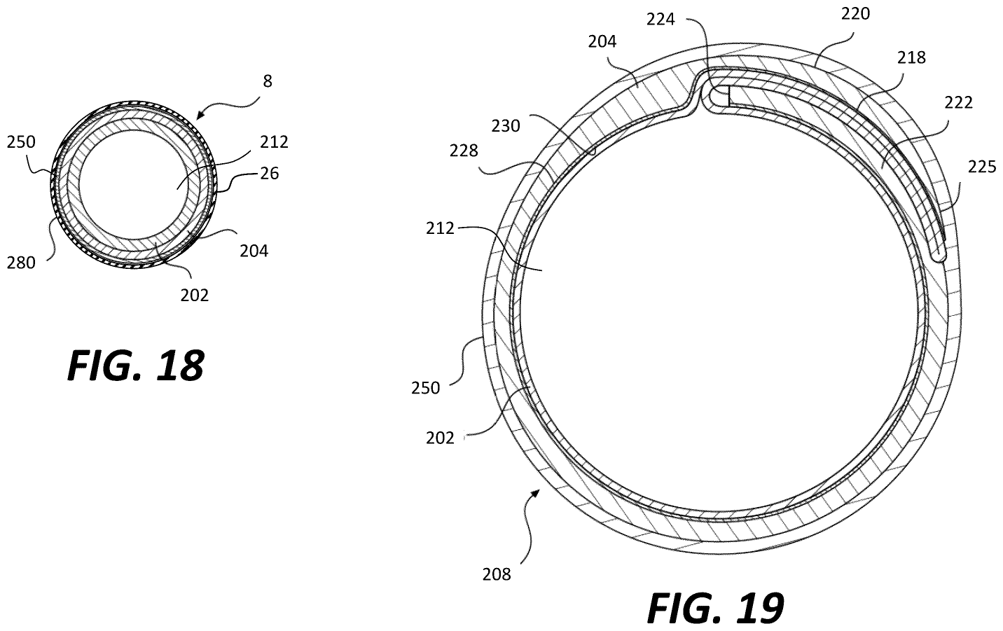

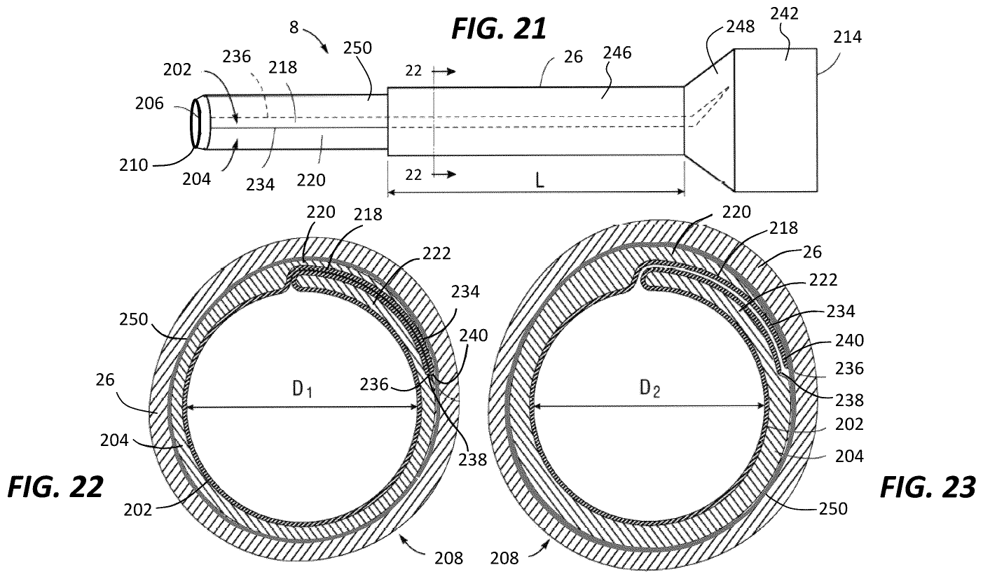

FIGS. 18 and 19 show additional cross sections taken at different points along the sheath 208. FIG. 18 shows a cross-section of a segment of the sheath near the proximal end 214 of the sheath 8, as indicated by section line 18-18 in FIG. 15. At this location, the sheath 8 includes the inner layer 202, outer layer 204, elastic outer layer 250/outer jacket, and the strain relief layer 26. At this location, near the proximal end of the sheath 8, the inner and outer layers 202, 204 are substantially tubular. Here the inner and outer layers 202, 204 can be formed without any slits or folded portions in the layers. By contrast, as described below, the inner and outer layers 202, 204 at different locations along the sheath 8 (e.g., at the point indicated by section line 19-19 in FIG. 15 and/or the point indicated by line section 22-22 in FIG. 21) can have a different configuration.

As shown in FIG. 19, the inner layer 202 can be arranged to form a substantially cylindrical lumen 212 therethrough. Inner layer 202 can include one or more folded portions 218. In the implementation shown in FIG. 19, inner layer 202 is arranged to have one folded portion 218 that can be positioned on either side of the inner layer 202. Inner layer 202 can be continuous, in that there are no breaks, slits, or perforations in inner layer 202. Outer layer 204 can be arranged in an overlapping fashion such that an overlapping portion 220 overlaps at least a part of the folded portion 218 of the inner layer 202. As shown in FIG. 19, the overlapping portion 220 also overlaps an underlying portion 222 of the outer layer 204. The underlying portion 222 can be positioned to underlie both the overlapping portion 220 of the outer layer 204, as well as the folded portion 218 of the inner layer 202. Thus, the outer layer 204 can be discontinuous, in that it includes a slit or a cut in order to form the overlapping and underlying portions 220, 222. In other words, a first edge 224 of the outer layer 204 is spaced apart from a second edge 225 of the outer layer 204 so as not to form a continuous layer.

As shown in FIG. 19, the sheath 8 can also include a thin layer of bonding or adhesive material 228 positioned between the inner and outer layers 202, 204. In one implementation, the adhesive material 228 can comprise a polyurethane material such as Tecoflex. The adhesive material 228 can be positioned on an inner surface 230 of at least a portion of the outer layer 204 so as to provide adhesion between selected portions of the inner and outer layers 202, 204. For example, the outer layer 204 may only include a Tecoflex layer (adhesive material 228) around the portion of the inner surface 230 that faces the lumen-forming portion of the inner layer 202. In other words, the Tecoflex layer can be positioned so that it does not contact the folded portion 218 of the inner layer 202 in some implementations. In other implementations, the Tecoflex layer can be positioned in different configurations as desired for the particular application. For example, as shown in FIG. 19, the Tecoflex layer can be positioned along the entire inner surface 230 of the outer layer 204. In an alternative example, the Tecoflex layer can be applied to the outer surface of the inner layer 202 instead of the inner surface of the outer layer 204. The Tecoflex layer can be applied to all or selected portions on the inner layer 202; for example, the Tecoflex layer can be formed only on the portion of the inner layer 202 that faces the lumen-forming portion of the outer layer 204 and not on the folded portion 218. The configuration of FIG. 19 allows for radial expansion of the sheath 208 as an outwardly directed radial force is applied from within (e.g., by passing a medical device such as a prosthetic heart valve through the lumen 212). As radial force is applied, the folded portion 218 can at least partially separate, straighten, and/or unfold, and/or the overlapping portion 220 and the underlying portion 222 of the outer layer 204 can slide circumferentially with respect to one another, thereby allowing the diameter of lumen 212 to enlarge.

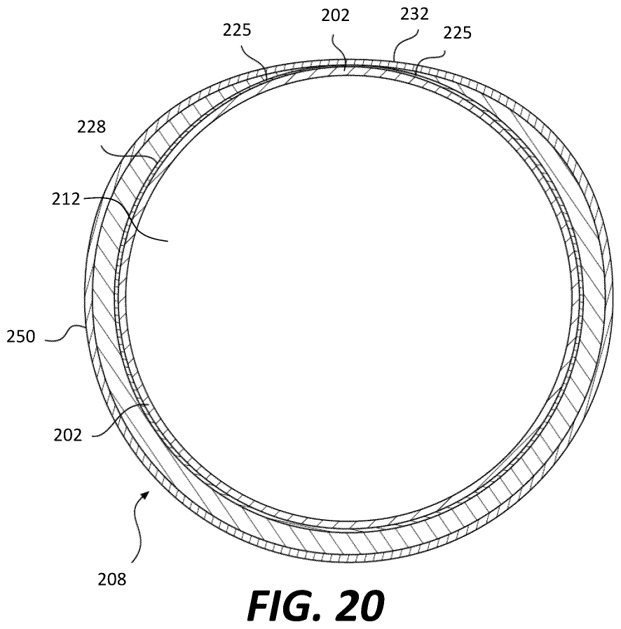

In this manner, the sheath 208 is configured to expand from a resting/unexpanded configuration (FIG. 19) to an expanded configuration shown in FIG. 20. In the expanded configuration, as shown in FIG. 20, an annular gap 232 can form between the longitudinal edges of the overlapping portion 220 and the underlying portion 222 of the outer layer 204. As the sheath 208 expands at a particular location, the overlapping portion 220 of the outer layer 204 can move circumferentially with respect to the underlying portion 222 as the folded portion 218 of the inner layer 202 unfolds. This movement can be facilitated by the use of a low-friction material for inner layer 202, such as PTFE. Further, the folded portion 218 can at least partially separate and/or unfold to accommodate a medical device having a diameter larger than that of lumen 212 in the resting/unexpanded configuration. As shown in FIG. 20, in some implementations, the folded portion of the inner layer 108 can completely unfold, so that the inner layer 108 forms a cylindrical tube at the location of the expanded configuration.

Similar to the example sheath 8 in FIG. 14, the sheath 8 is configured to locally expands at a particular location corresponding to the location of the medical device along the length of the lumen 212, and then locally contracts once the medical device has passed that particular location. Thus, a bulge may be visible, traveling longitudinally along the length of the sheath 8 as a medical device is introduced through the sheath 8, representing continuous local expansion and contraction as the device travels the length of the sheath 8. Each segment of the sheath 8 will locally contract after removal of any radial outward force such that the sheath 8 at least partially returns to the original resting/unexpanded diameter of lumen 212. Similar to the example sheath described above, an elastic outer layer 250 can (optionally) be provided along the sheath 8, urging the inner and outer layers 202, 204 back towards the unexpanded configuration.

The layers 202, 204 of sheath 8 can be configured having the folded portion 218 as shown in FIG. 19 along at least a portion of the length of the sheath 208. In some examples, the inner and outer layers 202, 204 can be configured as shown in FIG. 19 along the length A (FIG. 15) such that the folded portion 218 extends from a location adjacent the soft tip portion 206 to a location closer to the proximal end 214 of the sheath 8, adjacent and/or under the distal end of the strain relief layer 26. In this matter, the sheath 8 is expandable and contractable only along a portion of the length of the sheath corresponding to length A (which typically corresponds to the section of the sheath inserted into the narrowest section of the patient's vasculature).

In some examples, the folded portion 218 portion extends from a location adjacent the soft tip portion 206 under the strain relief layer 26, as illustrated in FIG. 21. In this example, the folded structure of the inner layer 202 extends from the tip portion 206, under the strain relief layer 26 and along the tapered portion 248 of the strain relief layer 26.

FIGS. 22 and 23 illustrate cross-section views of the sheath 8 taken along the strain relief layer 26 at section line 22-22 in FIG. 21. In this example, the folded portion 218 of the inner layer 202 extends under the strain relief layer 26. FIG. 22 shows a cross-section of the sheath 8 in a resting/unexpanded configuration having an inner diameter D₁. FIG. 23 shows a cross-section of the sheath 208 in a (partially) expanded configuration, having an inner diameter D₂, where D₂ is greater than D₁.

As shown in FIGS. 22-23, in some examples, the overlapping portion 220 does not overlap the entire folded portion 218 of the inner layer 202, and thus a portion of the folded portion 218 can be directly adjacent to the strain relief layer 26 in locations where the strain relief layer 26 is present. In locations where the strain relief layer 26 is not present, part of the folded portion 218 may be visible from the outside of the sheath 8, as seen in FIG. 21 (and/or visible through an elastic outer layer 250 described in more detail below). In these examples, the sheath 8 can include a longitudinal seam 234 where the overlapping portion 220 terminates at the folded portion 218. In use, the sheath 8 can be positioned such that the seam 234 is posterior to the point of the sheath that is 180 degrees from the seam 234 (e.g., facing downward in the view of FIG. 21). As shown in FIG. 21, the seam 234 need not extend the entire length of the sheath 8, and end at a transition point between portions of the sheath having a folded inner layer and portions of the sheath not having a folded inner layer.

In some examples, the folded portion 218 can include a weakened portion 236, such as a longitudinal perforation, score line, and/or slit, along at least a portion of the length of the inner layer 202. The weakened portion 236/slit allows for the two adjacent ends 238, 240 of the folded portion 218/inner layer 202 to move relative to one another as the sheath 8 expands to the expanded configuration shown in FIG. 23. For example, the sheath 8 locally expands as a medical device is inserted therethrough, causing the weakened portion 236 to split/separate.

In each of the example sheaths 8 described above, the sheath 8 may include an elastic outer layer 250 that expands with the sheath 8. The elastic outer layer 250 can provide an inwardly directed radial force that directs the sheath towards a folded/unexpanded configuration. Similar to the strain relief layer 26, elastic outer layer 250 can also provide hemostasis (e.g., prevent blood loss during implantation of the prosthetic device).

The elastic outer layer 250 can be positioned around at least a portion of the strain relief layer 26, outer layer 108, 204 and/or the inner layers of the sheath 8. As illustrated in FIGS. 21-23, the outer layer 250 can surround the entire circumference of outer layer 204, and can extend longitudinally along any portion of the length of the sheath 8, including along (over or under) the strain relief layer 26. The elastic outer layer 250 extends for a length along at least a portion of the main body of the sheath 8. In some examples, the elastic outer layer 250 extends to a point adjacent the distal end 210, or can extend all the way to the distal end 210 of sheath 8. For example, the elastic outer layer 250 extends over the entire length of the sheath 8.

As shown in FIGS. 17-20, 22 and 23, the elastic outer layer 250 can be a continuous tubular layer, without slits or other discontinuities. The elastic outer layer 250 extends between strain relief layer 26 and the outer surface of the outer layer 204. In other examples, the elastic outer layer 250 extends over the outer surface of the strain relief layer 26 and the outer surface of the outer layer 204. In further examples, the elastic outer layer 250 extends both over the strain relief layer 26 and/or between the outer layer of the sheath 8 and the strain relief layer 26.

The elastic outer layer 250 can comprise any pliable, elastic material(s) that expand and contract, preferably with a high expansion ratio. Preferably, the materials used can include low durometer polymers with high elasticity, such as Pebax, polyurethane, silicone, and/or polyisoprene. Materials for the elastic outer layer 250 can be selected such that it does not impede expansion of the inner and outer layers of the sheath 8. The elastic outer layer 250 can have a thickness ranging from, for example, about 0.001″ to about 0.010″. In some implementations, the elastic outer layer 250 can have a thickness of from about 0.003″ to about 0.006″. The elastic outer layer 250 can be configured to stretch and expand as the sheath expands, as shown in the expanded configuration in FIG. 20.

As illustrated in FIGS. 2, 15 and 21, the sheath 8 includes a strain relief layer 26. The strain relief layer 26/tube is provided adjacent the proximal end of the sheath 8 and extends along/over the outer surface of the sheath 8. In some examples, the strain relief layer 26 is provided over the outer layer 108, 204 of the sheath 8. The strain relief layer 26 forms a smooth transition between the sheath hub 20 and the sheath 8 and facilitates mating of the sheath 8 with the sheath hub 20.

Additionally, and as will be described in more detail below, the strain relief layer 26 provides a region of higher durometer or stiffness that restricts expansion of the underlying sheath layers. This helps to ensure hemostasis between the portions of the sheath 8 inside the patient and the sheath hub (external to the patient). The increased durometer and/or stiffness along the strain relief layer 26 prevents blood from flowing between the various layers of the sheath 8 exterior to the patient during the procedure, helping to withstand the blood pressure that would otherwise cause the sheath to “balloon up” with body fluid/blood. Additionally, the strain relief layer 26 can be sized and configured to form a seal with the patient's artery when inserted, such that blood is substantially prevented from flowing between the strain relief layer 26 and the vessel wall. For example, although the strain relief layer 26 does not extend all the way to the distal end 210 of the sheath 8, the strain relief layer 26 can extend distally enough along the sheath 8 that when the sheath 8 is fully inserted into the patient a portion of the strain relief layer 26 extends through and seals against the arteriotomy site.

As described above, the strain relief layer 26 is provided over the outer layer 108, 204 of the sheath 8. The strain relief layer 26 can be bonded to the outer layer 108, 204 to prevent the strain relief layer 26 from sliding over the outer layer and “bunching up” in response to the friction forces applied by the surrounding tissue during insertion of the sheath 8 into the patient's vasculature. For example, the strain relief layer 26 can be bonded at the proximal end and/or distal end of the outer layer 108, 204. At the proximal and distal ends, the strain relief layer 26 can be bonded to the outer layer 204 around the full circumference of the outer layer. At the distal end of the sheath 208, the strain relief layer 26 can alternatively be bonded to the inner layer(s) of the sheath 8. For example, the strain relief layer 26 can be bonded to the distal end surface of the inner layer 102, 202.

FIGS. 18, 22 and 23 illustrate cross-section views of the sheath 8 along the strain relief layer 26. FIG. 18 shows a cross-section of a segment of the sheath near the proximal end 214 of the sheath 8, as indicated by line 18-18 in FIG. 15. Similarly, FIGS. 22 and 23 show cross-section segments of various example sheaths near the proximal end 214 of the sheath 8 and closer to the distal end of the strain relief layer 26, as indicated by section line 22-22 in FIG. 21. As illustrated in each of FIGS. 15-23, the sheath 8 at this location can comprise an inner layer (liner) 202, outer layer 204, adhesive material 228 layer, an optional elastic outer layer 250, and the strain relief layer 26.

The strain relief layer 26 extends circumferentially around at least a portion of the inner layer 202 and outer layer 204. The strain relief layer 26 extends from the proximal end 214 of the sheath 8 towards the distal end 210 of the sheath 8. As shown in FIG. 21 (and FIG. 15), the strain relief layer 26 extends for a length L along at least a portion of the main body of the sheath 8. In further examples, the strain relief layer 26 extends to a point adjacent the distal end 210, or can extend all the way to the distal end 210 of sheath 8. In some examples, the longitudinal length L of the strain relief layer 26 can range from about 10 cm to the entire length of the sheath 8.

The strain relief layer 26 extends to/adjacent the proximal end 214 of the sheath 8 and provides a compression fit over the distal end of the sheath hub 20 thereby coupling the sheath 8 to the sheath hub 20. Additionally, or alternatively, the strain relief layer 26 secured between the sheath hub 20 and the sheath hub cap 22 or other fastening device for by coupling the proximal end of the sheath to the sheath hub 20. In some examples, the strain relief layer 26 does not extend all the way to the proximal end 214 of the sheath 208.

It is understood that strain relief layer 26, as shown herein, can have similar composition and characteristics of the inner and outer layers as disclosed herein. Various compositions are disclosed, for example, in Application No. PCT/US2021/301275, entitled “Expandable sheath for introducing an endovascular delivery device into a body,” the disclosure of which is herein incorporated by reference.

The strain relief layer 26 can comprise any lubricious, low-friction, and/or relatively non-elastic material. Preferably the materials used can include high durometer polymers, with low elasticity. In some examples, the strain relief layer 26 is composed of the same and/or similar material to the inner layer 202 and/or outer layer 204. For example, as described above regarding the inner and/or outer layer 102, 108, exemplary materials can include polyurethane (e.g., high density polyethylene), ultra-high-molecular-weight polyethylene (UHMWPE) (e.g., Dyneema®), high-molecular-weight polyethylene (HMWPE), or polyether ether ketone (PEEK). Other suitable materials for the strain relief layer 26 can include polyimide, polytetrafluoroethylene (PTFE), expanded polytetrafluoroethylene (ePTFE), ethylene tetrafluoroethylene (ETFE), nylon, polyethylene, polyamide, polyether block amide (e.g., Pebax), and/or combinations of any of the above. Materials for the strain relief layer 26 can be selected such that it impedes expansion of the underlying layers of the sheath 8.

The strain relief layer 26 can have a thickness ranging from, for example, about 0.001″ to about 0.010″. In some implementations, the strain relief layer 26 can have a thickness of from about 0.003″ to about 0.006″. The wall thickness is measured radially between the inner surface of the strain relief layer 26 and the outer surface of the strain relief layer 26.

In alternative examples, the material composition and/or wall thickness can change along the length of the strain relief layer 26. For example, the strain relief layer 26 can be provided with one or more segments, where the composition and/or thickness changes from segment to segment. In an example aspect, the Durometer rating of the composition changes along the length of the strain relief layer 26 such that segments near the proximal end comprise a stiffer material or combination of materials, while segments near the distal end comprise a softer material or combination of materials. Similarly, the wall thickness of the strain relief layer 26 in segments near the proximal end can be thicker/greater than the wall thickness of the elastic outer layer 250 near the distal end.

As illustrated in FIGS. 15, 21 and 24, the strain relief layer 26 has a proximal end and a distal end and a central lumen extending longitudinally therethrough. The strain relief layer 26 includes a generally tubular shaped proximal portion 242 adjacent the proximal end of the strain relief layer 26, and a generally tubular shaped distal portion 246 adjacent the distal end of the strain relief layer 26. The strain relief layer 26 includes a frustoconical shaped tapered portion 248 extending between the proximal portion 242 and the distal portion 246 of the strain relief layer 26, such that the diameter of the strain relief layer 26 at the proximal portion 242 is greater than the diameter of the strain relief layer 26 at the distal portion 246 of the strain relief layer 26. The tapered portion 248 and the flared proximal portion 242 help ease the transition of the medical device/delivery system when passing between the larger diameter sheath hub 20 to the smaller diameter of the sheath 8.

As described above, the strain relief layer 26 is made of a material that is stiffer than the other sheath 8 layers such that the strain relief layer 26 inhibits expansion of the portion of the sheath disposed along/under the strain relief layer 26. Because radial expansion is limited along the strain relief layer 26, higher push forces are necessary to advance the medical device (implant 12) through the central lumen of the sheath 8. In some examples, the highest push force through the sheath 8 is experienced near the ends (e.g., proximal and distal ends) of the strain relief layer 26.

In some aspects, the thickness and/or composition of the strain relief layer 26 can be adjusted to improve the performance of the strain relief layer 26 and to reduce the push force.

As described herein, pre-dilating the sheath 8, or a portion thereof, can help to reduce push forces required to insert the medical device/delivery system through the central lumen of the sheath 8. Pre-dilating the sheath 8 releases and/or loosens any bonding or adhesion of the sheath 8 layers that occurs during the manufacturing process, e.g., bonding between the inner and outer layers 202, 204, bonding between the folded portion 218 and outer layer 204, bonding between the inner/outer layers and the strain relief layer 26. Pre-dilating can also break or separate the weakened portion 236 of folded portion 218 of the inner layer 202, separating adjacent ends 238, 240 of the folded portion 218, as described above and illustrated in FIG. 23. With the sheath 8 layers able to move freely with respect to the other, the medical device/delivery system is pushed through the sheath 8 lumen at a much lower force.

In some instances, the sheath 8 is pre-dilated by passing a relatively large dilator (e.g., 22 French dilator) into the sheath 8 and through the strain relief layer 26. This can be done during sheath 8 preparation, prior to sheath 8 insertion into the patient and/or with the sheath 8 at least partially inserted into the patient. However, this method requires significant physical strength of the user (i.e., grip and arm strength) to advance the dilator into the strain relief layer 26. Additionally, it is challenging to control the dilation distance. It is important that the dilator not pass significantly beyond the distal end of the strain relief layer 26 to avoid splitting the main body of the sheath 8 beyond the distal end of the strain relief layer 26. Expanding/dilating the portion of the sheath 8 beyond the end of the strain relief layer 26 can cause irregular sheath 8 expansion because, frequently, the expanded portion of the sheath 8 does not recover smoothly to/toward the original unexpanded configuration and this results in difficulty or vessel injury during insertion, movement and/or withdraw of the pre-dilated sheath 8 in the vasculature. Current methods for controlling the desired dilation length of the sheath 8 and/or strain relief layer 26 is prone to user error and/or inaccuracies because it relies on a user's visual observation of the dilator as it passes through the strain relief layer 26 and stopping advancement just when the portion of the sheath 8 beyond the distal end of the strain relief layer 26 starts to expand. This manual method is inherently difficult to train, difficult to enforce proper technique, and prone to errors.

In the example sheath system described herein, a solution to remove human error is to control and/or adjust the length of the dilator, ensuring that any expansion forces provided by the dilator do not extend beyond the distal end 342 of the strain relief layer 26. However, due to the nature of many sheath 8 manufacturing processes, the length of the strain relief layer 26 can be variable between sheaths. For example, in some systems, the length of the strain relief layer 26 can vary +/−15 mm. This wide tolerance makes it difficult to design a single shortened dilator that can be used to reliably and consistently dilate the desired length of the strain relief layer 26 of a given sheath 8, i.e., dilate the entire length of the strain relief layer 26 without passing into the unexpanded sheath 8 beyond the distal end 342 of the strain relief layer 26. This requires the dilator length be determined and adjusted on a case-by-case basis based on the length of the strain relief layer 26 of a given sheath 8.

The devices, systems, and methods described below provide for system including an adjustable length dilator that can be used to determine and set the desired length of the dilator based on a given strain relief layer 26 such that the dilator length can be quickly and accurately set to pre-dilate the correct portion of a sheath 8.

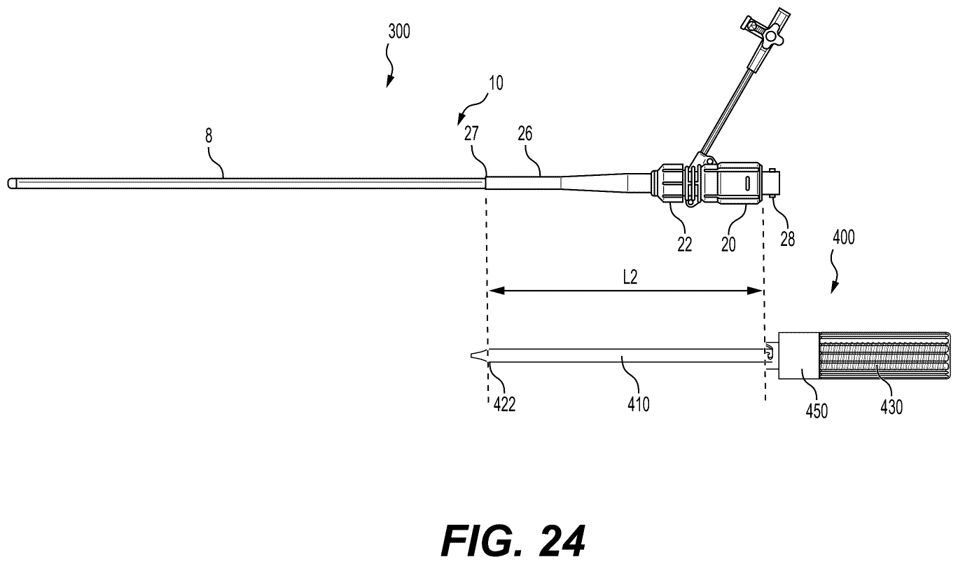

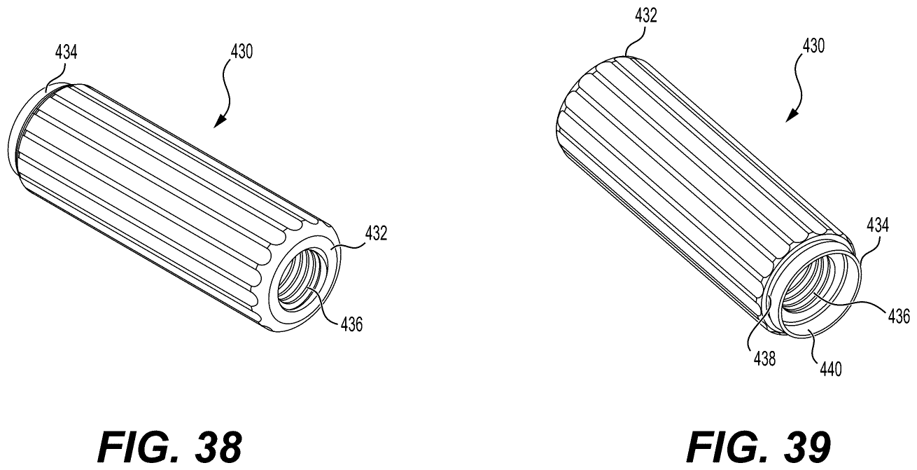

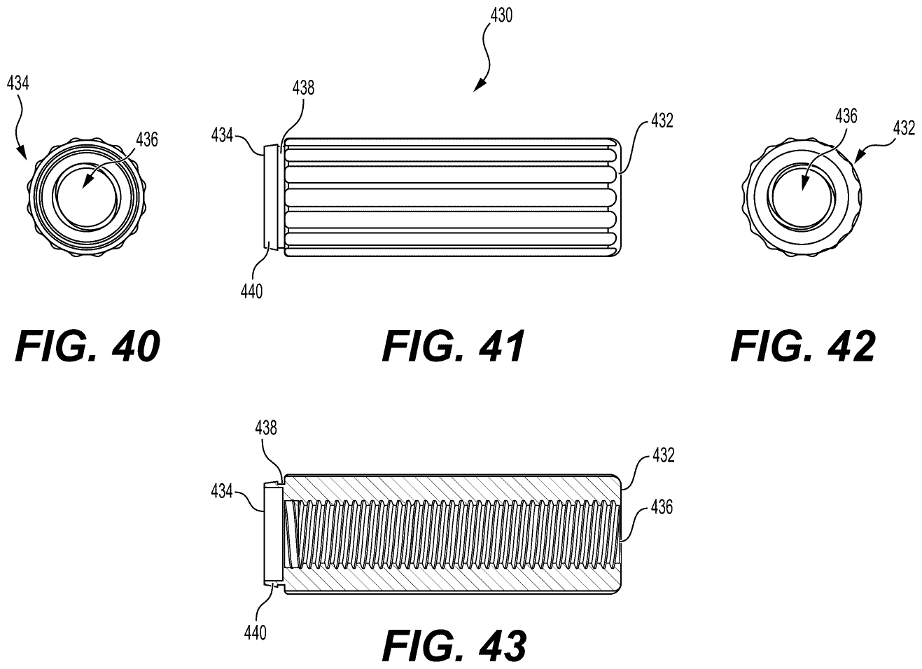

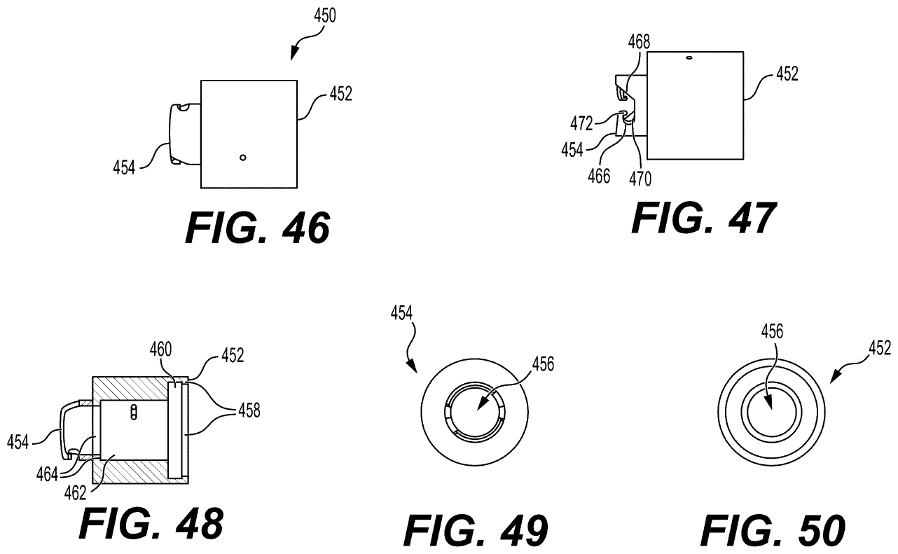

FIGS. 24-52 show an example sheath system 300 with an adjustable length dilator 400. The sheath system 300 includes a radially expandable sheath 8 as described herein and an adjustable length dilator 400. The expandable sheath 8 and sheath hub 20 shown in FIG. 24 correspond to other expandable sheath configurations described herein, e.g., the layered configuration illustrated in FIGS. 11-14 and/or FIGS. 15-23. As described above, in some aspects, the sheath 8 includes a continuous inner layer (e.g., inner layer 102, 202) defining a central lumen extending through the sheath 8, and a tubular strain relief layer 26 extending along the outer surface of the sheath 8 that limits radial expansion of the portion of the sheath 8 proximate the sheath hub 20. As described above, the various layers of the sheath 8 and the strain relief layer 26 are configured to locally expand from an unexpanded configuration, at a first diameter, to an expanded configuration at a second, larger, diameter, due to the outwardly directed radial force exerted on the lumen of the sheath (e.g., inner layer 202) by the dilator and/or a medical device, and then locally contract at least partially back to the unexpanded configuration as the dilator and/or medical device passes through the lumen.

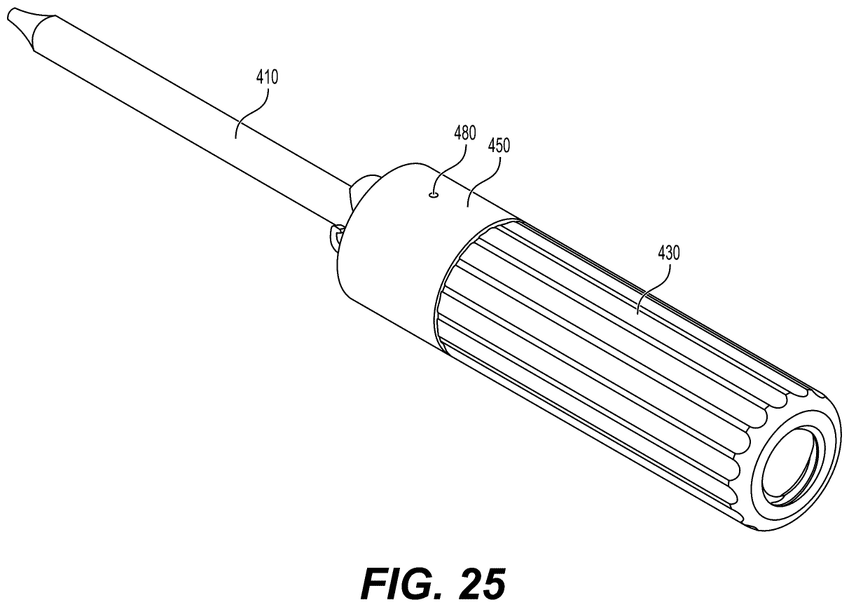

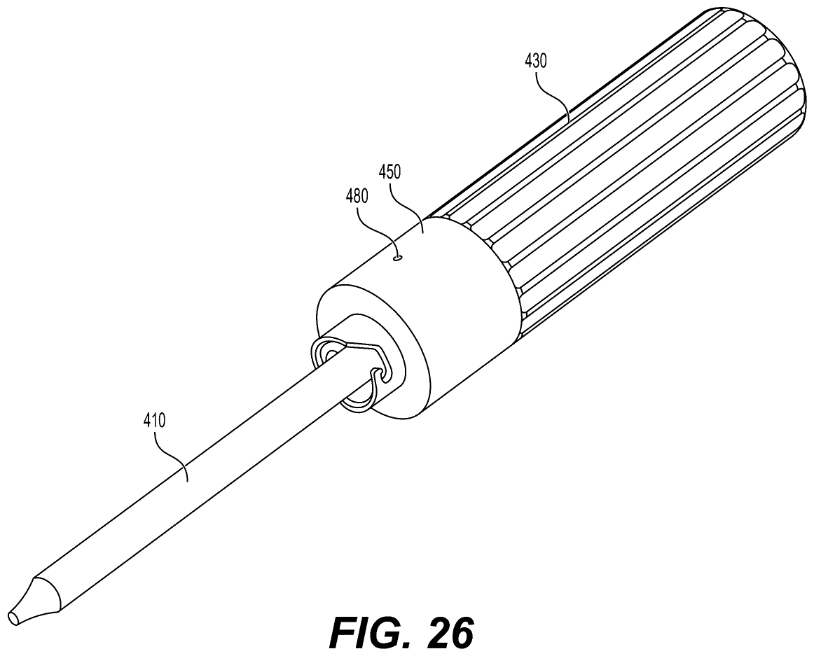

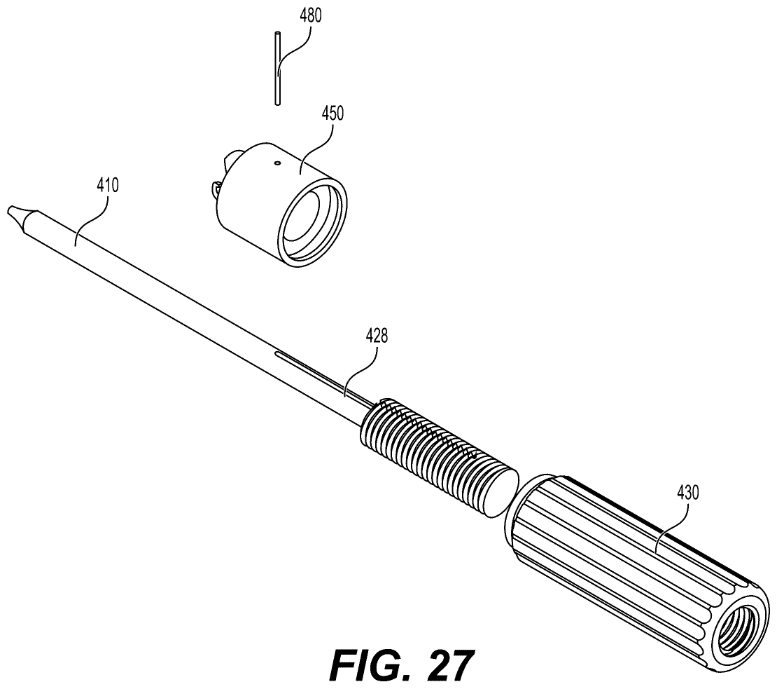

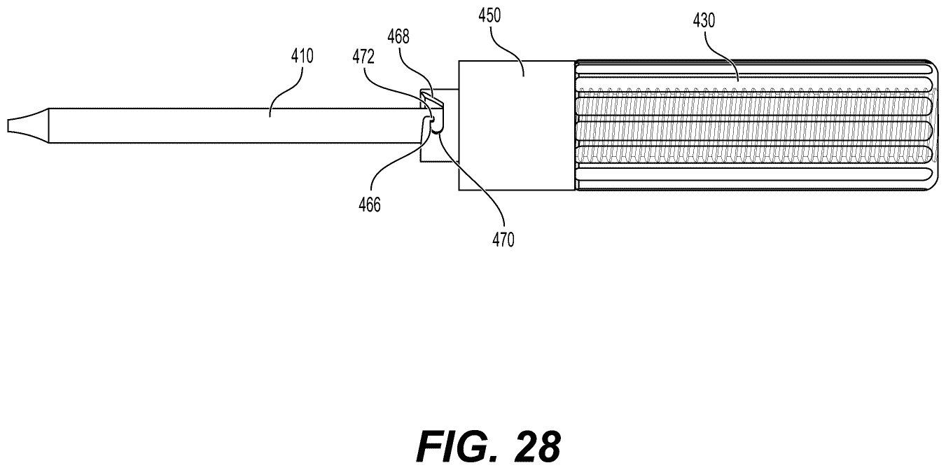

FIGS. 24-25 show proximal and distal perspective views of the example dilator 400, FIG. 27 is an exploded perspective view of the dilator 400, and FIG. 28 shows a side view of the dilator 400. The dilator 400 is sized and configured to be received within and expand the central lumen of the sheath 8. The dilator 400 includes a dilator shaft 410, a knob 430 coupled to a proximal end 412 of the dilator shaft 410, a dilator hub 450 (e.g., coupler) rotatably coupled to the knob 430. In some implementations, a pin 480 is used to couple the dilator hub 450 with the dilator shaft 410 and prevent rotational movement therebetween.

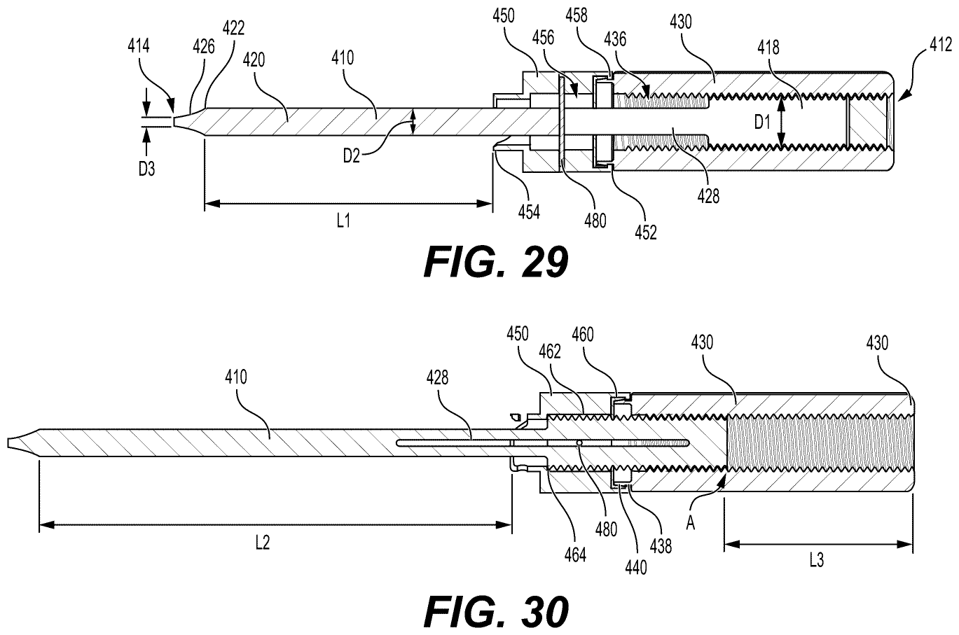

As will be described in more detail below, rotational movement of the knob 430 results in a corresponding axial movement of the dilator shaft 410 in a direction along a longitudinal axis of the dilator 400. FIG. 29 shows a cross-sectional view of the dilator 400 in a retracted position and FIG. 30 shows a cross-sectional view of the dilator 400 in an extended position. In some implementations, rotational movement of the knob 430 is used to adjust a length of the dilator shaft 410 extending beyond the distal end of dilator hub 450 and/or the length of the dilator shaft 410 extending beyond the distal end of the knob 430. For example, rotational movement of the knob 430 in a first direction causes the dilator shaft 410 to move axially in a first direction, and rotational movement of the knob 430 in a second, opposite, direction, causes the dilator shaft 410 to move axially in a second, opposite direction. In some implementations, rotational movement of the knob 430 in a clockwise direction results in a corresponding distal axial movement of the dilator shaft 410, and rotational movement of the knob 430 in a counterclockwise direction results in corresponding proximal movement of the dilator shaft 410.

As described above, the dilator shaft 410 is moveable between a first (retracted) position shown in FIG. 29, and a second (extended) position shown in FIG. 30. As provided in FIGS. 29 and 30, the length of the dilator shaft 410 (L2) in the second position is greater than the length of the dilator shaft 410 (L1) in the first position. In some implementations, the length of the dilator shaft 410 is measured along the elongated body portion 420 between the distal end 454 of the dilator hub 450 and the proximal end of the tapered distal end 424 of the dilator shaft 410, at a location corresponding to the distal end of the portion of the dilator shaft 410 that provides the outwardly directed radial force for expanding the sheath 8. In further implementations, the length of the dilator shaft 410 is measured along the elongated body portion 420 between the distal end 454 of the dilator hub 450 and the distal end 414 of the dilator shaft 410. In some implementations, the length of the dilator shaft (L2) in the second position ranges from 4 inches to 6 inches. For example, in some implementations, the length of the dilator shaft (L2) in the second position is 5 inches. As will be described in more detail below, when the dilator shaft 410 is received within the central lumen of the sheath 8, the length of the dilator shaft 410 extending within the sheath 8 can be adjusted (e.g., from the first position to the second position) to expand a corresponding length of the sheath 8 (and/or strain relief layer 26).

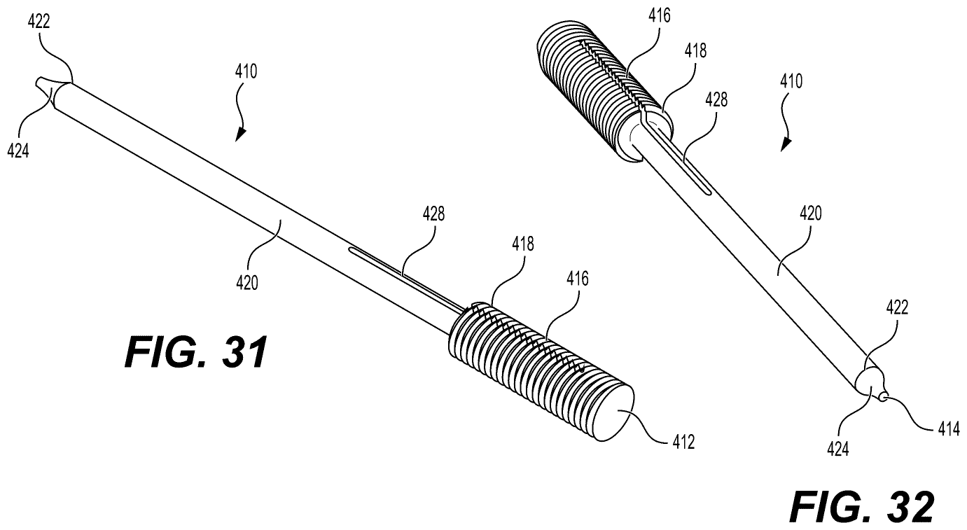

FIGS. 31-37 illustrate the dilator shaft 410. The dilator shaft 410 includes an elongated body portion 420 extending between the proximal end 412 and distal end 414 of the dilator shaft 410. When the dilator shaft 410 is received within the sheath 8, the elongated body portion 420 exerts a radially outward radial force on the lumen of the sheath (e.g., inner layer 202), thereby locally expanding the various layers of the sheath 8, including the strain relief layer 26.

In some aspects, the dilator shaft 410 includes a central lumen extending therethrough that can be used, for example, to receive a guide wire.

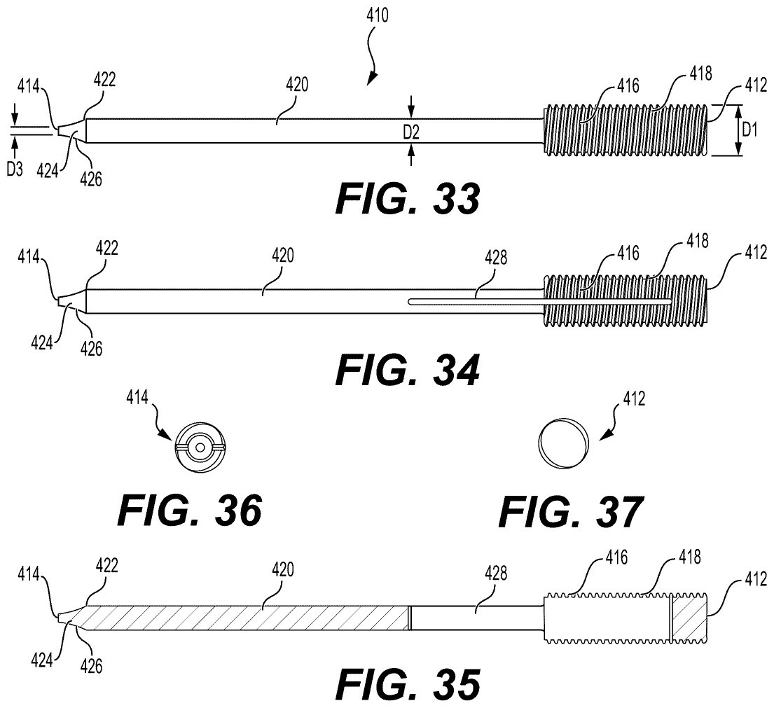

The elongated body portion 420 includes a threaded outer surface 416 received within and threadingly coupled to threaded central lumen 436 of the knob 430. In some implementations, the dilator shaft 410 includes an increased diameter portion 418 adjacent the proximal end 412, where the threaded outer surfaced 416 is provided along at least a portion of the increased diameter portion 418. As noted in FIG. 33, the elongated body portion 420 extends between the increased diameter portion 418 and the distal end 414 of the dilator shaft 410, where the increased diameter portion 418 has a diameter (D1) greater than a diameter (D2) of the elongated body portion 420. In some implementations, the diameter (D2) of the elongated body portion 420 ranges from 16 French to 28 French. In further implementations, the diameter (D2) of the elongated body portion 420 ranges from 12 French to 22 French. In further aspects, the elongated body portion 420 has a diameter ranging from 14 French to 24 French. In some aspects, the diameter of the elongated body portion 420 is 22 French.

In some implementations, the dilator shaft 410 includes an expansion element projecting from the outer surface of the dilator shaft 410, the expansion element can include a regular or irregular shaped projection extending from the outer surface (e.g., around all or a portion of the circumference) of the dilator shaft 410. In some implementations, when the dilator 350 is received within the sheath 8, the expansion element 365 exerts a radially outward radial force on the lumen of the sheath (e.g., inner layer 202), thereby locally expanding the various layers of the sheath 8, including the strain relief layer 26. In some aspects, the diameter of the expansion element ranges from 16 French to 28 French, from 12 French to 24 French, from 14 French to 24 French, and/or from 14 French to 22 French. In some implementations, the diameter of the expansion element is 22 French.

In some aspects, the dilator shaft 410 includes a tapered distal end 424. For example, the dilator shaft 410 can include a tapered distal end 424 having decreasing taper toward the distal end 414 of the dilator shaft 410 such that the tapered distal end 424 extends from the elongated body portion 420 to the distal end 414 of the dilator shaft 410. As illustrated in FIG. 31, the tapered distal end 424 can include a flattened or rounded distal most end surface (tip) such that the tapered distal end 424 tapers from the diameter (D2) of the elongated body portion 420 to a distal end diameter (D3), where the distal end diameter (D3) less than the diameter (D2) of the elongated body portion 420. In some implementations, the tapered distal end 424 of the dilator shaft 410 tapers continuously the distal most end surface (tip). In some implementations, the tapered distal end 424 includes a smooth tapering surface. In further implementations, the tapered distal end 424 includes a concave tapered surface 426. In other implementations, the tapered distal end 424 includes a convex surface tapering surface, and/or any other regular or irregularly shaped tapered surface.

FIGS. 38-43 illustrate the knob 430. The knob 430 is coupled to the proximal end 412 of the dilator shaft 410. As will be described in more detail below, rotational movement of the knob 430 results in a corresponding axial movement of the dilator shaft 410 in a direction along a longitudinal axis of the dilator 400. The knob 430 is threadingly coupled to the proximal end 412 of the dilator shaft 410 such that rotational movement between the knob 430 and the dilator shaft 410 results in the corresponding axial movement of the dilator shaft 410 between the first (retracted) position (FIG. 29) and the second (extended) position (FIG. 30). FIG. 43 shows a longitudinal cross section of the knob 430 taken along the longitudinal axis of the knob 430. As illustrated in FIG. 43, the knob 430 includes a threaded central lumen 436 extending at least partially therethrough. In some implementations, the threaded central lumen 436 extends between and/or from the proximal end 432 to a distal end 434 of the knob 430. As illustrated in FIGS. 29-30, the dilator shaft 410 includes a threaded outer surface 416 that is received within and threadingly coupled to threaded central lumen 436 of the knob 430.

In some implementations, the length of travel of the dilator shaft 410 within the threaded central lumen 436 of the knob 430 corresponds to a screw travel length (L3), shown in FIG. 30. The screw travel length (L3) is measured between the proximal end 432 of the knob 430 and a location (A) within the threaded central lumen 436 when the dilator shaft 410 is in the second position. In some implementations, when the dilator shaft 410 is in second position at least four threads of the dilator shaft 410 are engaged with the threaded central lumen 436. In further implementations, implementations, when the dilator shaft 410 is in second position at least two threads of the dilator shaft 410 are engaged with the threaded central lumen 436. In some implementations, the screw travel length (L3) ranges from 1 inch to 3 inches. In some aspects, the screw travel length (L3) is 2 inches.

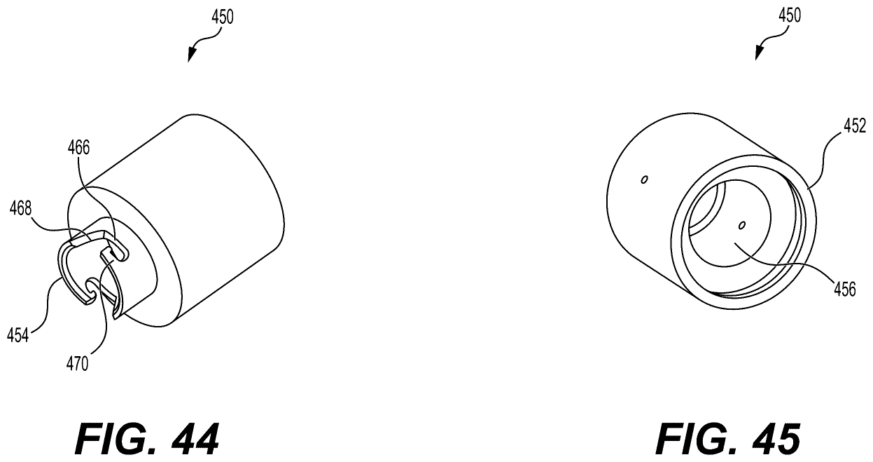

A dilator hub 450 and/or coupler is rotatably coupled to the knob 430. Various aspects of the dilator hub 450 are illustrated in FIGS. 44-48. The dilator hub 450 includes a central lumen 456 extending between the proximal end 452 end the distal end 454 of the dilator hub 450. When assembled, the dilator shaft 410 extends through the central lumen 456 of the dilator hub 450. In some implementations, the dilator shaft 410 is axially and rotatably movable within the central lumen 456 of the dilator hub 450.

The dilator hub 450 is rotatably coupled to the knob 430. In some implementations, the dilator hub 450 can freely rotate with respect to the knob 430. As illustrated in FIGS. 29-30, the dilator hub 450 includes a clip for rotatably coupling the dilator hub 450 to the knob 430. For example, the dilator hub 450 includes a shoulder 458 extending radially inward from the central lumen 456. The shoulder 458 is received within a corresponding recess 438 provided on the knob 430 such that the shoulder 458 rotates within/around the recess 438. In some implementations, the shoulder 458 freely rotates within the recess 438 around the entire circumference of the knob 430.

As illustrated in FIGS. 42 and 43, the recess 438 is provided on a projection 440 extending axially from a distal end 434 of the knob 430. In some implementations, the projection 440 includes a tapered outer surface, e.g., a reducing tapered outer surface, configured to assist with advancing the projection 440 beyond the shoulder 458 of the dilator hub 450 and securing the shoulder 458 within the recess 438.

As illustrated in FIG. 48, the central lumen 456 of the dilator hub 450 includes a first diameter portion 460 and a second diameter portion 462, where the diameter of the first diameter portion 460 is greater than a diameter of the second diameter portion 462. The shoulder 458 is provided on the first diameter portion 460 and adjacent a proximal end 452 of the dilator hub 450. As provided in FIGS. 29 and 30, the axial length of the first diameter portion 460 corresponds to at least the length of the projection 440. In some implementations, as shown in FIG. 48, the central lumen 456 of the dilator hub 450 includes a third diameter portion 464, where the diameter of the third diameter portion 464 is less than the diameter of the second diameter portion 462.

As shown in FIG. 30, the diameter of the second diameter portion 462 is greater than the diameter of the increased diameter portion 418 of the dilator shaft 410, e.g., the diameter of the threaded outer surface 416, such that the dilator shaft 410 is axially movable within the second diameter portion 462 without engaging the dilator hub 450. In some implementations, the third diameter portion 464 forms a second shoulder within the central lumen 456 of the dilator hub 450 such that interference between the second shoulder (third diameter portion 464) and the distal end of the increased diameter portion 418 of the dilator shaft 410 prevents axial movement of the dilator shaft 410 within the dilator hub 450, fixing the distal post position of the dilator shaft 410 with respect to the dilator hub 450/knob 430.

The dilator hub 450 includes engagement features for fixing the dilator 400 to the sheath 9/sheath hub 20. Similar to the locking channel 38 provided on the introducer locking hub body 32 illustrated in in FIG. 9B, the dilator hub 450 includes a locking channel 466 for coupling the dilator hub 450 to the sheath hub 20 via the locking sleeve 28 (illustrated in FIGS. 2-6). As shown in FIG. 47, the locking channel 466 extends from the distal end 454 of the dilator hub 450 axially towards the proximal end 452 of the dilator hub 450 and circumferentially around the dilator hub 450. The locking channel 466 includes a guide portion 468 that extends at an angle between the opening on the distal end surface of the dilator hub 450 and the locking portion 470 extending circumferentially around the dilator hub 450. Like locking channel 38, upon rotation of the dilator hub 450 and/or rotation of the sheath locking sleeve 28, the guide portion 468 of the locking channel 466 is sized and configured to direct a corresponding projection provided on the locking sleeve 28 (e.g., guide 31) in an axial direction along the side wall of the guide portion 468 towards the locking portion 470. The locking portion 470 of the locking channel 466 is configured to securely engage the corresponding projection (e.g., guide 31) and fix the axial position of the dilator hub 450 with respect to the sheath locking sleeve 28/sheath hub 20. In some implementations, similar to locking sleeve 28, the locking portion 470 of the locking channel 38 includes a catch 472 extending from a sidewall of the locking portion 470. The catch 472 secures the guide 31 within the locking portion 470 of the locking channel 466.

In some implementations the dilator shaft 410 is coupled to the dilator hub 450 via a pin 480 that extends through the dilator shaft 410 and into the dilator hub 450. For example, as illustrated in FIGS. 29-32 and 34-35, the dilator shaft 410 includes an elongated slot 428 extending radially through at least a portion of the dilator shaft 410. The slot 428 extends axially along a portion of the length of the dilator shaft 410. In some implementations, the slot 428 extends through the entire thickness/width of the dilator shaft 410. In some implementations, the slot 428 is coplanar with the longitudinal axis of the dilator shaft 410.

As shown in FIG. 29, the pin 480 extends from a first side wall of the dilator hub 450, through the slot 428, and into a second sidewall of the dilator hub 450. Engagement between the pin 480 and the slot 428 restricts rotational movement between the dilator hub 450 and the dilator shaft 410. In some implementations, engagement between the pin 480 and the slot 428 limits axial movement of the dilator shaft 410 with respect to the dilator hub 450. For example, engagement between the proximal and distal ends of the slot 428 with the pin 480 limits movement of the dilator shaft 410 with respect to the dilator hub 450 along the longitudinal axis of the dilator shaft 410.

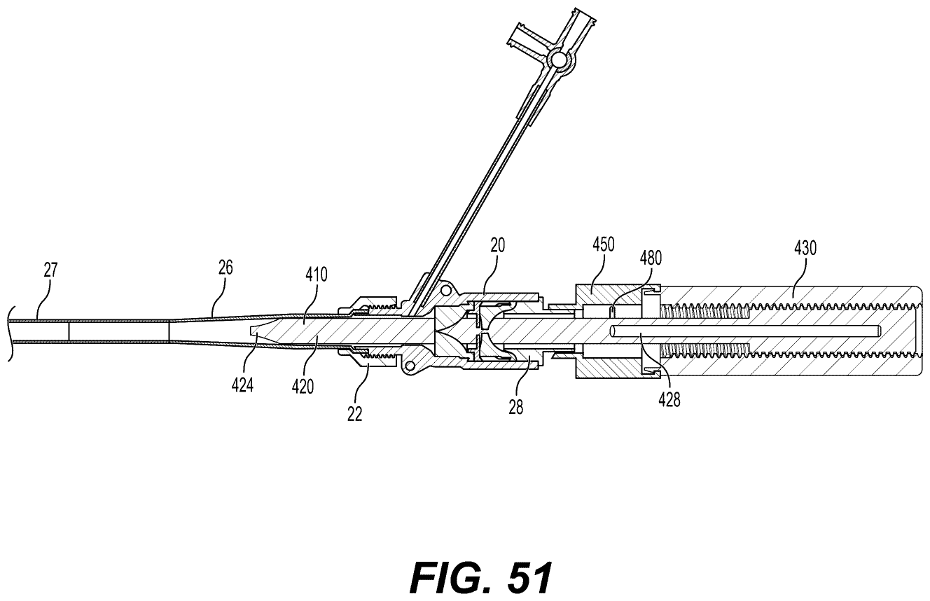

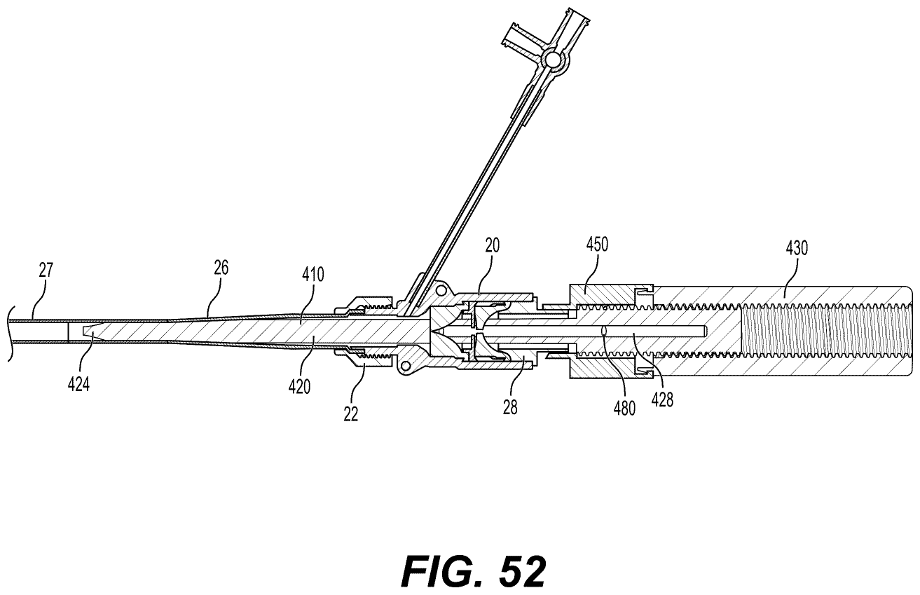

FIGS. 51 and 52 illustrate the dilator 400 received within the central lumen of the sheath 8. The method of adjusting the length of the dilator 400/dilator shaft 410 to expand/pre-dilate the sheath 8 is described below. However, it is contemplated that the length of the dilator 400/dilator shaft 410 can be adjusted without coupling the dilator 400 to the sheath 8 and/or advancing the dilator shaft 410 into the central lumen of the sheath 8.

The method includes providing a sheath 8 according to any of the examples described above. The sheath 8 includes a continuous inner layer (e.g., inner layers 102, 104, 106, 202) defining a central lumen extending therethrough and a tubular strain relief layer 26 provided over the inner layer at a proximal end of the sheath 8 and extending along at least a portion of a length of the sheath 8. As described above, the strain relief layer 26 provides a region of higher durometer or stiffness that restricts expansion of the underlying sheath layers. For example, in some implementations, the strain relief layer 26 comprises a material having a higher durometer than the sheath 8 such that the strain relief layer 26 restricts expansion of the sheath 8. In some examples, the sheath 8 includes an outer layer (e.g., fourth layer 108, outer layer 204) provided over the inner layer, and under or over the tubular strain relief layer 26. In some examples, the inner layer includes at least one folded portion, as described above in reference to FIGS. 11-23, that expands/unfolds as the sheath 8 expands. The strain relief layer 26 extends along at least a portion of a length of the sheath 8 from the proximal end of the sheath 8 toward the distal end.

The dilator 400 is coupled to the sheath 8 by advancing the distal end 414 of the dilator shaft 410 at least partially within the central lumen of the sheath 8. The dilator shaft 410 is sized and configured to be received (e.g., slidably and/or rotatably received) within the central lumen of the sheath 8 and sheath hub 20. The dilator shaft 410 is advanced within the sheath 8 until the dilator hub 450 is positioned adjacent the proximal end of the sheath locking sleeve 28 such that the guide 31 projecting from the outer surface of the sheath locking sleeve 28 is received within the locking channel opening on the dilator hub 450. The dilator hub 450 is rotated in a first direction with respect to the locking sleeve 28 (and/or sheath hub 20) to move the guide 31 along the locking channel 466 into a locked position. Continued rotation of the dilator hub 450 causes movement of the guide 31 along the locking channel 466 into a locked position. The guide 31 first moves along the guide portion 468 of the locking channel 466 toward the locking portion 470 of the locking channel 466. As illustrated in FIG. 47, the guide portion 468 of the locking channel 466 extends in a direction from the distal end 454 of the dilator hub 450 axially towards the proximal end 452 of the dilator hub 450, and the locking portion 470 extends circumferentially around the dilator hub 450. Further rotation of the dilator hub 450 directs the guide 31 into the locking portion 470 of the locking channel 466, where the locking portion 470 is configured to securely engage the guide 31 and fix the axial position of the dilator hub 450 with respect to the sheath locking sleeve 28. In some implementations, the locking portion 470 includes a catch 472 that secures the guide 31 within the locking portion 470 of the locking channel 466. In this example, rotation of the dilator hub 450 in the first direction causes the guide 31 to overcome the bias force of the catch 472 and advance the guide 31 beyond the catch 472 in the locking portion 470, where the catch 472 secures the guide 31 within the locking portion 470 and fixes the axial location of the sheath with respect to the dilator hub 450. When coupled, the central lumen of the sheath 8 is aligned with the central lumens of the sheath hub 20, the sheath locking sleeve 28, and the dilator hub 450.

With the dilator shaft 410 received within the central lumen of the sheath 8, the dilator shaft 410 can be moved between the first (retracted) position as shown in FIG. 51, and second (extended) position, shown in FIG. 52, where the length (L2) of the dilator shaft 410 extending into the sheath 8 in the second position is greater than the length (L1) of the dilator shaft 410 extending into the sheath 8 in the first position.

The length of the dilator shaft 410 is adjusted by rotating the knob 430 causing the threaded outer surface 416 of the dilator shaft 410 to threadingly engage the threaded central lumen 436 of the knob 430 and results in a corresponding axial movement of the dilator shaft 410 in a direction along the longitudinal axis of the dilator 400. The knob 430 is rotatably coupled with respect to the dilator hub 450 (which is in turn coupled to the sheath hub 20 via the sheath locking sleeve 28 as described above). However, the knob 430 is coupled to the dilator hub 450 (via the shoulder 458 and recess 438 structure of the dilator hub 450 and knob 430, respectively) such that the axial position between the two remains constant as the dilator shaft 410 moves axially within the sheath 8.

The pin 480 extends from the dilator hub 450 through the slot 428 extending radially through the dilator shaft 410 such that engagement between the pin 480 and the slot 428 restricts rotational movement between the dilator hub 450 and the dilator shaft 410. As a result, rotating the knob 430 causes the pin 480 to slidingly engage the slot 428 and guides axial movement of the dilator shaft 410 while limiting rotational movement of the dilator shaft 410 with respect to the dilator hub 450. Accordingly, as the knob 430 is rotated in a first direction, the dilator shaft 410 engages the knob 430 and resulting in a corresponding axial movement of the dilator shaft 410 in a distal direction along a longitudinal axis of the dilator 400 thereby increasing a length of the dilator shaft 410 received within the central lumen of the sheath 8.

As the length of the dilator shaft 410 extending within the sheath 8 is adjusted/increased, a corresponding length of the sheath 8 is locally expanded from an unexpanded configuration at a first diameter to the expanded configuration at a second, larger, diameter. Similarly, when the length of the dilator shaft 410 extending within the sheath 8 is adjusted/increased, a corresponding length of the strain relief layer 26 is locally expanded from an unexpanded configuration at a first diameter to an expanded configuration at a second, larger, diameter.

In some implementations, when the dilator shaft 410 is received within the central lumen of the sheath the second position, the dilator shaft 410 extends along a length of the sheath corresponding to the strain relief layer 26. In further implementations, when the dilator shaft 410 is received within the central lumen of the sheath 8 in the second position, the dilator shaft 410 extends along a length of the sheath corresponding to a majority of the (length) strain relief layer 26 (e.g., see FIG. 52). In some implementations, when the dilator shaft 410 is received within the central lumen of the sheath 8 in the second position, the dilator shaft 410 extends along a length of the sheath 8 corresponding to an entire length of the strain relief layer 26 (e.g., see FIG. 24). In some implementations, the length of the dilator shaft 410 extending into the sheath 8 when in the second position corresponds to the entire length of the strain relief layer 26 including a tolerance amount. For example, including a tolerance amount of +/−15 mm. As shown in FIG. 24, in some implementations, in the second position the distal end 422 of the elongated body portion 420 of the dilator shaft 410 is aligned with a distal end 27 of the strain relief layer 26. As shown, the dilator shaft 410 includes a tapered distal end 424 and is in the second position with respect to the dilator hub 450, the distal end 422 of the elongated body portion 420 aligns with the distal end of the strain relief layer 26 and the tapered distal end 424 extends beyond the distal end 27 of the strain relief layer 26. Because the tapered distal end 424 has a diameter less than the diameter of the elongated body portion 420, the tapered distal end 424 does not expand the portion of the sheath beyond the strain relief layer 26.

The sheath 8 and strain relief layer 26 expand in response to the outwardly directed radial force exerted against the central lumen of the sheath 8 by the dilator shaft 410. In some implementations, the outer diameter of the dilator shaft 410 is greater than the inner diameter of the sheath 8 along the length corresponding to the strain relief layer 26, as such, movement of the dilator shaft 410 within the central lumen of the sheath 8 causes the sheath 8 and the corresponding portion of the strain relief layer 26 to radially expand. In some implementations, as the strain relief layer 26 moves from the unexpanded to the expanded configuration, the length of the strain relief layer 26 remains constant.

Is it contemplated, that the sheath 8 and strain relief layer 26 may also locally expand in response to the outwardly directed radial force exerted against the central lumen of the sheath 8 by a passing medical device. It is contemplated that in some implementations, only the sheath 8 expands in response to the movement of the dilator shaft 410 (and/or medical device) passing through the sheath. It is further contemplated that in another implementation, only the strain relief layer 26 expands in response to the movement of the dilator shaft 410 (and/or medical device) passing through the sheath.

Once the desired length of the sheath 8/strain relief layer 26 is expanded the dilator 400 is withdrawn from the central lumen of the sheath 8 and the sheath 8/strain relief layer 26 at least partially (locally) contracts back toward the unexpanded configuration. In some aspects, the sheath 8 and/or strain relief layer 26 are radially biased in an inward direction. In further aspects, the sheath 8 includes an outer elastic layer 250 providing a radially inward force that directs the sheath 8 and strain relief layer 26 to/toward the unexpanded configuration.

In some implementations, the knob 430 is rotated in a second direction opposite to the first direction, causing the dilator shaft 410 to engage the knob 430 and resulting in a corresponding axial movement of the dilator shaft 410 in a proximal direction along the longitudinal axis of the dilator 400 toward the first (retracted) position. As a result, the length of the dilator shaft 410 received within the central lumen of the sheath 8 is reduced.

With the dilator shaft 410 withdrawn from the central lumen of the sheath 8 a desired amount, the dilator 400 can be uncoupled from the sheath 8/sheath hub 20/sheath locking sleeve 28. The dilator 400 is uncoupled from the sheath 8 by rotating the dilator hub 450 in a second direction with respect to the locking sleeve 28 to slide the guide 31 along the locking channel 466 into the unlocked position, similar to how the introducer locking hub 30 is uncoupled from the sheath locking sleeve 28 as described above. For example, rotating the dilator hub 450 in the second direction causes the guide 31 to overcome the bias force of the catch 472 and advance from the locking portion 470 to the guide portion 468 of the locking channel 466. Further rotation of the dilator hub 450 in the second direction causes the guide 31 to side along the locking channel 466, from the locking portion 470 toward the guide portion 468. Further rotation of the dilator hub 450 in the second direction directs the guide 31 out of the locking portion 470 of the locking channel 466 and through the guide portion 468 to release the dilator hub 450 from the sheath locking sleeve 28.

With the dilator hub 450 uncoupled from the sheath locking sleeve 28, the dilator 400 can be removed from the sheath 8.

When used to insert a medical device to a treatment site within patient, the sheath 8 is inserted at least partially into the blood vessel of a patient before and the distal end of the sheath 8 is positioned at a location proximate the treatment site. While pre-dilating the sheath 8 is described in advance of inserting the sheath 8 into the patient, in some aspects, the sheath 8 is inserted into the patient before the pre-dilation steps.

With the dilator 400 removed, a medical device is introduced into the proximal end of the central lumen of the sheath 8. Because the sheath 8 and strain relief layer 26 have been pre-dilated/expanded, the push forces necessary to advance the medical device through the sheath 8/strain relief layer 26 are reduced compared to a non-dilated sheath.

The medical device can then be advanced into the sheath 8, particularly the portion of the sheath 8 including the strain relief layer 26. As the medical device is advanced through the portion of the sheath 8 corresponding to the strain relief layer 26, the medical device exerts an outwardly directed radial force against the central lumen of the sheath 8, causing the sheath 8 and the strain relief layer 26 (and corresponding portion of the sheath 8) proximate the medical device to locally expand from an unexpanded configuration to an expanded configuration. In some examples, the medical device is contracted or compressed radially as it passes through the strain relief layer 26, from the proximal portion 242, through the tapered segment 248 and into the smaller diameter distal portion 246. As the medical device pass through the sheath 8/strain relief layer 26, the sheath 8/strain relief layer 26 locally contract towards the unexpanded configuration.

The medical device is then advanced beyond the distal end 27 of the strain relief layer 26, into the lumen of the longitudinally body portion of the sheath 8 (beyond the strain relief layer 26). As the medical device is advanced through the sheath 8 beyond the strain relief layer 26, the sheath 8 locally expands from the unexpanded configuration to the expanded configuration at a location proximate the medical device in response to the outwardly directed radial force of the medical device exerted against the inner layer/central lumen of the sheath 8.

As the medical device passes through the lumen of the sheath 8, the sheath 8 locally contracts at least partially back to the unexpanded configuration when the medical device has passed. When used to deliver a medical device to a treatment site within a patient, the medical device is then passed through the distal tip 9/distal opening of the sheath 8 and delivered to the treatment site. The position of the medical device can be moved or adjusted until the medical device is adequately positioned within the patient. With the medical device delivered to the treatment site, delivery system/components coupled to the medical device are then removed from the medical device and withdrawn from the lumen of the sheath 8. The sheath 8 is removed from the patient and the opening in the blood vessel and skin closed.

In some implementations, at least one of the inner layer and/or outer layer includes at least one folded portion, e.g., ridges 126 and valleys 128 of the fourth (outer) layer 108 of the sheath 8 illustrated in FIGS. 11-14, and folded portion 218 of the inner layer 202 of the sheath 8 illustrated in FIGS. 15-23. Locally expanding the lumen of the sheath 8 causes a length of the folded portion to at least partially unfold. Similarly, locally contracting the sheath 8 at least partially back to the unexpanded configuration causes a length of the folded portion to urge back towards a folded configuration.

In some implementations, the outer layer is a discontinuous outer layer and includes an overlapping portion (e.g., overlapping portion 220) and an underlying portion (e.g., underlying portion 220). When the sheath 8 is in the unexpanded configuration, the overlapping portion overlaps the underlying portion with the folded portion of the inner layer disposed between the overlapping portion and the underlying portion (FIGS. 17, 19, 22, 23). As the sheath 8 locally expands to/toward the expanded configuration, a length of the overlapping portion moves circumferentially with respect to the underlying portion unfolding. As illustrated in FIG. 20, when the sheath 8 is fully expanded, the inner layer extends into the gap 232 formed between the longitudinal edges of the overlapping portion 220 and the underlying portion 222 of the outer layer 204.

In some implementations, the sheath 8 includes an elastic outer layer 250 that extends at least partially over the outer layer and/or the strain relief layer 26. The elastic outer layer 250 locally expands and contracts as the medical device is advanced through the lumen of the sheath 8. In some examples, the elastic outer layer 250 urges the various layers of the sheath 8 toward an unexpanded configuration.

In some aspects, the medical device is a prosthetic device mounted in a radially crimped state on a delivery apparatus, and advancing the prosthetic device through the lumen of the sheath includes advancing the delivery apparatus and the prosthetic device through lumen of the sheath 8 and into a vasculature of the patient. In some implementations, the prosthetic device comprises a prosthetic heart valve and the method further comprises implanting the prosthetic heart valve at a treatment site within the patient. In some implementations, the prosthetic heart valve is mounted on a balloon catheter of the delivery apparatus as the prosthetic heart valve is advanced through the sheath 8. In further implementations, the sheath 8 is inserted into a femoral artery of the patient.

### Exemplary Aspects

In view of the described processes and compositions, hereinbelow are described certain more particularly described aspects of the disclosures. These particularly recited aspects should not, however, be interpreted to have any limiting effect on any different claims containing different or more general teachings described herein, or that the “particular” aspects are somehow limited in some way other than the inherent meanings of the language and formulas literally used therein.

Example 1. A dilator including: a dilator shaft; a knob coupled to a proximal end of the dilator shaft; a dilator hub rotatably coupled to the knob; and a pin coupling the dilator hub with the dilator shaft and preventing rotational movement therebetween, wherein rotational movement of the knob results in a corresponding axial movement of the dilator shaft in a direction along a longitudinal axis of the dilator.

Example 2. The dilator according to any example herein, particularly example 1, wherein rotational movement of the knob is used to adjust a length of the dilator shaft extending beyond a distal end of dilator hub and/or a distal end of the knob, wherein the rotational movement of the knob in a first direction causes the dilator shaft to move axially in a first direction, and rotational movement of the knob in a second, opposite, direction, causes the dilator shaft to move axially in a second, opposite direction.

Example 3. The dilator according to any example herein, particularly examples 1-2, wherein the dilator shaft is moveable from a first position (retracted) to a second position (extended), where a length (L2) of the dilator shaft in the second position is greater than a length (L1) of the dilator shaft in the first position.

Example 4. The dilator according to any example herein, particularly examples 1-3, wherein the knob is threadingly coupled to the proximal end of the dilator shaft such that rotational movement between the knob and the dilator shaft results in the corresponding axial movement of the dilator shaft.

Example 5. The dilator according to any example herein, particularly example 4, wherein the knob includes a threaded central lumen 436 extending at least partially therethrough, and wherein the dilator shaft includes a threaded outer surface received within and threadingly coupled to threaded central lumen of the knob.

Example 6. The dilator according to any example herein, particularly examples 1-5, wherein the dilator shaft includes an increased diameter portion adjacent the proximal end, where a threaded outer surfaced provided along at least a portion of the increased diameter portion.

Example 7. The dilator according to any example herein, particularly examples 1-6, wherein the dilator shaft includes an elongated body portion extending between the increased diameter portion and a distal end of the dilator shaft, where the increased diameter portion has a diameter (D1) greater than a diameter (D2) of the elongated body portion.

Example 8. The dilator according to any example herein, particularly example 7, wherein the dilator shaft includes a tapered distal end extending from the distal end of the dilator shaft to the elongated body portion, wherein the tapered distal end tapers from the diameter (D2) of the elongated body portion to a distal end diameter (D3), where the distal end diameter (D3) less than the diameter (D2) of the elongated body portion.

Example 9. The dilator according to any example herein, particularly examples 1-8, wherein the dilator hub is rotatably coupled to the knob, wherein the dilator hub includes a central lumen extending through the dilator hub and a shoulder extending radially inward from the central lumen of the dilator hub, the shoulder is received within a corresponding recess provided on the knob, wherein the central lumen of the dilator hub includes a first diameter portion and a second diameter portion, where a diameter of the first diameter portion is greater than a diameter of the second diameter portion, wherein the shoulder is provided on the first diameter portion.

Example 10. The dilator according to any example herein, particularly example 9, wherein the central lumen of the dilator hub includes a third diameter portion, where a diameter of the third diameter portion is less than a diameter of the second diameter portion, wherein the diameter of the second diameter portion is greater than the diameter of an increased diameter portion of the dilator shaft such that the dilator shaft is axially movable within the second diameter portion, wherein the third diameter portion forms a second shoulder within the central lumen of the dilator hub such that interference between the second shoulder and a distal end of the increased diameter portion prevents axial movement of the dilator shaft within the dilator hub.

Example 11. The dilator according to any example herein, particularly examples 1-10, wherein the dilator hub includes a locking channel that extends from the distal end of the dilator hub axially towards a proximal end of the dilator hub and circumferentially around the dilator hub.

Example 12. The dilator according to any example herein, particularly example 11, wherein the locking channel includes a guide portion that extends at an angle between an opening on a distal end surface of the dilator hub and a locking portion that extends circumferentially around the dilator hub.

Example 13. The dilator according to any example herein, particularly examples 1-12, wherein the dilator shaft includes an elongated slot extending radially through at least a portion of the dilator shaft, where the slot extends axially along a portion of a length of the dilator shaft, wherein the pin extends from a first side wall of the dilator hub, through the slot, and into a second sidewall of the dilator hub, wherein engagement between the pin and the slot restricts rotational movement between the dilator hub and the dilator shaft.

Example 14. A sheath system comprising: a radially expandable sheath including: a continuous inner layer defining a central lumen extending therethrough, the inner layer having at least one folded portion; and a tubular strain relief layer provided along the inner layer positioned at a proximal end of the sheath and extending along at least a portion of a length of the sheath; and a dilator for expanding at least a portion of the sheath, the dilator including: a dilator shaft sized and configured to be received within the central lumen of the sheath; a knob coupled to a proximal end of the dilator shaft; a dilator hub rotatably coupled to the knob; and a pin coupling the dilator hub and the dilator shaft and preventing rotational movement therebetween, wherein rotational movement of the knob results in a corresponding axial movement of the dilator shaft in a direction along a longitudinal axis of the dilator to adjust a length of the dilator shaft received within the central lumen of the sheath, wherein at least a portion of the strain relief layer is configured to locally expand from an unexpanded configuration at a first diameter to an expanded configuration at a second, larger, diameter, and then locally contract at least partially back to the unexpanded configuration in response to an outwardly directed radial force exerted against the central lumen by the dilator shaft, and then locally contract at least partially back to the unexpanded configuration as the dilator shaft moves within the central lumen of the sheath.

Example 15. The sheath system according to any example herein, particularly example 14, wherein rotational movement of the knob is used to adjust a length of the dilator shaft extending beyond a distal end of dilator hub and/or a distal end of the knob, wherein the dilator shaft is moveable from a first position (retracted) to a second position (extended), where a length (L2) of the dilator shaft in the second position is greater than a length (L1) of the dilator shaft (L1) in the first position.

Example 16. The sheath system according to any example herein, particularly example 15, wherein, when the dilator shaft is received within the central lumen of the sheath and in the second position, the dilator shaft extends along a length of the sheath corresponding to the strain relief layer.

Example 17. The sheath system according to any example herein, particularly examples 15-16, wherein in the second position a distal end of the elongated body portion of the dilator shaft is aligned with a distal end of the strain relief layer.

Example 18. The sheath system according to any example herein, particularly examples 14-17, wherein the dilator hub is rotatably coupled to the knob, wherein the dilator hub includes a locking channel sized and configured to couple the dilator hub to the sheath, wherein the locking channel includes a guide portion that extends between an opening on a distal end surface of the dilator hub and a locking portion of the locking channel, the locking portion extending in a direction circumferentially around the dilator hub.

Example 19. The sheath system according to any example herein, particularly example 18, further including: a sheath hub fixedly coupled to the proximal end of the sheath, the sheath hub including a central lumen extending therethrough and coaxial with the lumen of the sheath, where the dilator shaft is sized and configured to be received within the central lumen of the sheath hub; a sheath locking sleeve removably coupled to the sheath hub, the sheath locking sleeve comprising a sleeve body having a proximal end and a distal end and defining a central lumen extending longitudinally between the proximal end and the distal end, a guide disposed on an outer surface of the sleeve body, where the guide is movable within the locking channel between an unlocked position where the sheath locking sleeve is rotationally and axially movable with respect to the dilator hub, and a locked position where the sheath locking sleeve is axially fixed with respect to the dilator hub.

Example 20. The sheath system according to any example herein, particularly example 19, wherein the guide portion of the locking channel is configured to direct the guide in an axial direction along a side wall of the guide portion towards the locking portion upon rotation of at least one of the dilator hub or the sheath locking sleeve, wherein the locking portion of the locking channel is configured to securely engage the guide fixing an axial position of the dilator hub with respect to the sheath locking sleeve.

Example 21. A method of adjusting a length of a dilator comprising: providing dilator comprising: a dilator shaft; a knob coupled to a proximal end of the dilator shaft; a dilator hub rotatably coupled to the knob; and a pin 480 coupling the dilator hub and the dilator shaft and preventing rotational movement therebetween, rotating the knob in a first direction causing the dilator shaft to engage the knob and resulting in a corresponding axial movement of the dilator shaft in a direction along a longitudinal axis of the dilator.

Example 22. The method according to any example herein, particularly example 21, wherein rotating the knob results in the dilator shaft is moving from a first position (retracted) to a second position (extended), where a length (L2) of the dilator shaft in the second position is greater than a length (L1) of the dilator shaft in the first position, wherein the knob includes a threaded central lumen extending at least partially therethrough and the dilator shaft includes a threaded outer surface received within and threadingly coupled to threaded central lumen of the knob, wherein rotating the knob causes the threaded outer surface of the dilator shaft to threadingly engage the threaded central lumen of the knob resulting in a corresponding axial movement of the dilator shaft in a direction along the longitudinal axis of the dilator.

Example 23. The method according to any example herein, particularly examples 21-22, wherein the pin extends from the dilator hub through a slot extending radially through the dilator shaft, wherein engagement between the pin and the slot restricts rotational movement between the dilator hub and the dilator shaft, wherein rotating the knob causes the pin to slidingly engage the slot guiding axial movement of the dilator shaft, and limiting rotational movement of the dilator shaft with respect to the dilator hub.

Example 24. The method according to any example herein, particularly examples 21-23, wherein the dilator hub is rotatably coupled to the knob such that the dilator hub can freely rotate with respect to the knob, wherein an axial location of the dilator hub with respect to the knob is fixed during rotation of the knob.

Example 25. A method of dilating a sheath comprising: providing a radially expandable sheath including: a continuous inner layer defining a central lumen therethrough, the inner layer having at least one folded portion; and tubular strain relief layer provided over the inner layer positioned at a proximal end of the sheath and extending along at least a portion of a length of the sheath, wherein at least a portion of the strain relief layer is configured to locally expand from an unexpanded configuration at a first diameter to an expanded configuration at a second, larger, diameter in response to an outwardly directed radial force exerted on the central lumen (e.g., by the dilator shaft received within the lumen of the inner layer), and then locally contract at least partially back to the unexpanded configuration as the outwardly directed radial force is removed from the central lumen (e.g., as the dilator moves within the lumen); providing a dilator for expanding at least a portion of the sheath, the dilator including: a dilator shaft sized and configured to be received within the central lumen of the expandable sheath; a knob coupled to a proximal end of the dilator shaft; a dilator hub rotatably coupled to the knob; a pin coupling the dilator hub and the dilator shaft and preventing rotational movement therebetween; coupling the dilator to the sheath; rotating the knob in a first direction causing the dilator shaft to engage the knob and resulting in a corresponding axial movement of the dilator shaft in a distal direction along a longitudinal axis of the dilator thereby increasing a length of the dilator shaft received within the central lumen of the sheath; advancing the dilator shaft through a portion of the central lumen of the sheath corresponding to the strain relief layer such that the dilator shaft exerts an outwardly directed radial force against the central lumen and causes the inner layer and the strain relief layer proximate the dilator shaft to locally expand from an unexpanded configuration to an expanded configuration; rotating the knob in a second direction opposite to the first direction, causing the dilator shaft to engage the knob and resulting in a corresponding axial movement of the dilator shaft in a proximal direction along the longitudinal axis of the dilator thereby reducing a length of the dilator shaft received within the central lumen of the sheath and removing the outwardly directed radial force exerted on the central lumen thereby causing the inner layer and/or the strain relief layer to locally contract at least partially back to the unexpanded configuration; uncoupling the dilator from the sheath; and removing the dilator from the sheath.

Example 26. The method according to any example herein, particularly example 25, wherein the dilator hub includes a locking channel sized and configured to couple the dilator hub to the sheath, the locking channel including a guide portion that extends between an opening on a distal end surface of the dilator hub and a locking portion of the locking channel, the locking portion extending in a direction circumferentially around the dilator hub, wherein the sheath includes a sheath locking sleeve provided at a proximal end of the sheath, the sheath locking sleeve comprising a sleeve body having a proximal end and a distal end and defining a central lumen extending longitudinally between the proximal end and the distal end, a guide disposed on an outer surface of the sleeve body, wherein coupling the dilator to the sheath includes: advancing a distal end of the dilator shaft at least partially within the central lumen of the sheath; positioning the dilator hub adjacent a proximal end of the sheath locking sleeve such that the guide projecting from an outer surface of the sheath locking sleeve is received within a locking channel opening on the dilator hub; and rotating the dilator hub in a first direction with respect to the locking sleeve to move the guide along the locking channel into a locked position.

Example 27. The method according to any example herein, particularly example 26, wherein movement of the guide along the locking channel into a locked position includes: movement of the guide along a guide portion of the locking channel toward a locking portion of the locking channel, where the guide portion of the locking channel extends in a direction from the distal end of the dilator hub axially towards the proximal end of the dilator hub and the locking portion extends circumferentially around the dilator hub; wherein further rotation of the dilator hub directs the guide into the locking portion of the locking channel, the locking portion configured to securely engage the guide and fix an axial position of the dilator hub with respect to the sheath locking sleeve.

Example 28. A method of inserting a medical device into a blood vessel of a patient, the method comprising: inserting a radially expandable sheath at least partially into the blood vessel of a patient, the sheath including: a continuous inner layer defining a central lumen therethrough, the inner layer having at least one folded portion; and tubular strain relief layer provided over the inner layer positioned at a proximal end of the sheath and extending along at least a portion of a length of the sheath, wherein at least a portion of the strain relief layer is configured to locally expand from an unexpanded configuration at a first diameter to an expanded configuration at a second, larger, diameter, and then locally contract at least partially back to the unexpanded configuration; coupling a dilator to the sheath, the dilator including: a dilator shaft sized and configured to be received within the central lumen of the expandable sheath; a knob coupled to a proximal end of the dilator shaft; a dilator hub rotatably coupled to the knob; a pin coupling the dilator hub and the dilator shaft and preventing rotational movement therebetween; rotating the knob in a first direction causing the dilator shaft to engage the knob and resulting in a corresponding axial movement of the dilator shaft in a distal direction along a longitudinal axis of the dilator thereby increasing a length of the dilator shaft received within the central lumen of the sheath; advancing the dilator shaft through a portion of the central lumen of the sheath corresponding to the strain relief layer such that the dilator shaft exerts an outwardly directed radial force against the central lumen and causes the inner layer and the strain relief layer proximate the dilator shaft to locally expand from an unexpanded configuration to an expanded configuration; rotating the knob in a second direction opposite to the first direction, causing the dilator shaft to engage the knob and resulting in a corresponding axial movement of the dilator shaft in a proximal direction along the longitudinal axis of the dilator thereby reducing a length of the dilator shaft received within the central lumen of the sheath and causing the inner layer and the strain relief layer to locally contract at least partially back to the unexpanded configuration; uncoupling the dilator from the sheath; removing the dilator from the sheath; introducing a medical device into a proximal end of the central lumen of the sheath; advancing the medical device through the sheath; and advancing the medical device beyond a distal opening in the sheath to a treatment site within the blood vessel.

Example 29. The method according to any example herein, particularly example 28, wherein advancing the medical device through the sheath includes: advancing the medical device through the portion of the sheath corresponding to the strain relief layer and thereby exerting an outwardly directed radial force by the medical device against the central lumen and causing the inner layer and the strain relief layer proximate the medical device to locally expand from an unexpanded configuration to an expanded configuration; locally contracting the strain relief layer towards the unexpanded configuration as the medical device passes through a corresponding portion of the sheath; advancing the medical device beyond a distal end of the strain relief layer; advancing a medical device through the lumen of the sheath causing the sheath to locally expand from the unexpanded configuration to the expanded configuration at a location proximate the medical device in response to the outwardly directed radial force of the medical device exerted against the inner layer; and locally contracting the sheath at least partially back to the unexpanded configuration as the medical device passes through the lumen.

Example 30. The method according to any example herein, particularly examples 28-29, wherein the medical device is a prosthetic device mounted in a radially crimped state on a delivery apparatus, wherein advancing the prosthetic device through the lumen of the sheath comprises advancing the delivery apparatus and the prosthetic device through lumen of the sheath and into a vasculature of the patient.

Example 31. The method according to any example herein, particularly example 30, wherein the prosthetic device comprises a prosthetic heart valve and the method further comprises implanting the prosthetic heart valve at a treatment site within the patient, wherein the prosthetic heart valve is mounted on a balloon catheter of the delivery apparatus as the prosthetic heart valve is advanced through the sheath.

In view of the many possible aspects to which the principles of the disclosed disclosure can be applied, it should be recognized that the illustrated aspects are only preferred examples of the disclosure and should not be taken as limiting the scope of the disclosure. Rather, the scope of the disclosure is defined by the following claims. We, therefore, claim as our disclosure all that comes within the scope and spirit of these claims.

What is claimed is:

1. A dilator including:
a dilator shaft;
a knob coupled to a proximal end of the dilator shaft;
a dilator hub rotatably coupled to the knob; and
a pin coupling the dilator hub with the dilator shaft and preventing rotational movement therebetween,
wherein rotational movement of the knob results in a corresponding axial movement of the dilator shaft in a direction along a longitudinal axis of the dilator.

2. The dilator of claim 1, wherein rotational movement of the knob is used to adjust a length of the dilator shaft extending beyond a distal end of dilator hub and/or a distal end of the knob,
wherein the rotational movement of the knob in a first direction causes the dilator shaft to move axially in a first direction, and rotational movement of the knob in a second, opposite, direction, causes the dilator shaft to move axially in a second, opposite direction,
wherein the dilator shaft is moveable from a first position (retracted) to a second position (extended), where a length (L2) of the dilator shaft in the second position is greater than a length (L1) of the dilator shaft in the first position.

3. The dilator of claim 1, wherein the knob is threadingly coupled to the proximal end of the dilator shaft such that rotational movement between the knob and the dilator shaft results in the corresponding axial movement of the dilator shaft.

4. The dilator of claim 1, wherein the knob includes a threaded central lumen extending at least partially therethrough,
wherein the dilator shaft includes a threaded outer surface received within and threadingly coupled to threaded central lumen of the knob, and where the dilator shaft includes an increased diameter portion adjacent the proximal end, where the threaded outer surface is provided along at least a portion of the increased diameter portion.

5. The dilator of claim 4, wherein the dilator shaft includes an elongated body portion extending between the increased diameter portion and a distal end of the dilator shaft, where the increased diameter portion has a diameter (D1) greater than a diameter (D2) of the elongated body portion,
wherein the dilator shaft includes a tapered distal end extending from the distal end of the dilator shaft to the elongated body portion, where the tapered distal end tapers from the diameter (D2) of the elongated body portion to a distal end diameter (D3), and the distal end diameter (D3) less than the diameter (D2) of the elongated body portion.

6. The dilator of claim 1, wherein the dilator hub is rotatably coupled to the knob, and the dilator hub includes a central lumen extending through the dilator hub and a shoulder extending radially inward from the central lumen of the dilator hub, where the shoulder is received within a corresponding recess provided on the knob,
wherein the central lumen of the dilator hub includes a first diameter portion and a second diameter portion, where a diameter of the first diameter portion is greater than a diameter of the second diameter portion,
wherein the shoulder is provided on the first diameter portion.

7. The dilator of claim 6, wherein the central lumen of the dilator hub includes a third diameter portion, where a diameter of the third diameter portion is less than a diameter of the second diameter portion,
wherein the diameter of the second diameter portion is greater than the diameter of an increased diameter portion of the dilator shaft such that the dilator shaft is axially movable within the second diameter portion,
wherein the third diameter portion forms a second shoulder within the central lumen of the dilator hub such that interference between the second shoulder and a distal end of the increased diameter portion prevents axial movement of the dilator shaft within the dilator hub.

8. The dilator of claim 1, wherein the dilator hub includes a locking channel that extends from a distal end of the dilator hub axially towards a proximal end of the dilator hub and circumferentially around the dilator hub.

9. The dilator of claim 1, wherein the dilator shaft includes an elongated slot extending radially through at least a portion of the dilator shaft, where the slot extends axially along a portion of a length of the dilator shaft,
wherein the pin extends from a first side wall of the dilator hub, through the slot, and into a second sidewall of the dilator hub,
wherein engagement between the pin and the slot restricts rotational movement between the dilator hub and the dilator shaft.

10. A sheath system comprising:
a radially expandable sheath including:
a continuous inner layer defining a central lumen extending therethrough, the inner layer having at least one folded portion; and
a tubular strain relief layer provided along the inner layer positioned at a proximal end of the sheath and extending along at least a portion of a length of the sheath; and
a dilator for expanding at least a portion of the sheath, the dilator including:
a dilator shaft sized and configured to be received within the central lumen of the sheath;
a knob coupled to a proximal end of the dilator shaft;
a dilator hub rotatably coupled to the knob; and
a pin coupling the dilator hub and the dilator shaft and preventing rotational movement therebetween,
wherein rotational movement of the knob results in a corresponding axial movement of the dilator shaft in a direction along a longitudinal axis of the dilator to adjust a length of the dilator shaft received within the central lumen of the sheath,
wherein at least a portion of the strain relief layer is configured to locally expand from an unexpanded configuration at a first diameter to an expanded configuration at a second, larger, diameter, and then locally contract at least partially back to the unexpanded configuration in response to an outwardly directed radial force exerted against the central lumen by the dilator shaft, and then locally contract at least partially back to the unexpanded configuration as the dilator shaft moves within the central lumen of the sheath.

11. The sheath system of claim 10, wherein rotational movement of the knob is used to adjust a length of the dilator shaft extending beyond a distal end of dilator hub and/or a distal end of the knob,
wherein the dilator shaft is moveable from a first position (retracted) to a second position (extended), where a length (L2) of the dilator shaft in the second position is greater than a length (L1) of the dilator shaft (L1) in the first position.

12. The sheath system of claim 11, wherein, when the dilator shaft is received within the central lumen of the sheath and in the second position, the dilator shaft extends along a length of the sheath corresponding to the strain relief layer.

13. The sheath system of claim 11, wherein in the second position a distal end of an elongated body portion of the dilator shaft is aligned with a distal end of the strain relief layer.

14. The sheath system of claim 10, wherein the dilator hub is rotatably coupled to the knob,
wherein the dilator hub includes a locking channel sized and configured to couple the dilator hub to the sheath,
wherein the locking channel includes a guide portion that extends between an opening on a distal end surface of the dilator hub and a locking portion of the locking channel, the locking portion extending in a direction circumferentially around the dilator hub.

15. The sheath system of claim 14, further including:
a sheath hub fixedly coupled to the proximal end of the sheath, the sheath hub including a central lumen extending therethrough and coaxial with the lumen of the sheath, where the dilator shaft is sized and configured to be received within the central lumen of the sheath hub;
a sheath locking sleeve removably coupled to the sheath hub, the sheath locking sleeve comprising a sleeve body having a proximal end and a distal end and defining a central lumen extending longitudinally between the proximal end and the distal end, a guide disposed on an outer surface of the sleeve body, where the guide is movable within the locking channel between an unlocked position where the sheath locking sleeve is rotationally and axially movable with respect to the dilator hub, and a locked position where the sheath locking sleeve is axially fixed with respect to the dilator hub.

16. The sheath system of claim 15, wherein the guide portion of the locking channel is configured to direct the guide in an axial direction along a side wall of the guide portion towards the locking portion upon rotation of at least one of the dilator hub or the sheath locking sleeve, wherein the locking portion of the locking channel is configured to securely engage the guide fixing an axial position of the dilator hub with respect to the sheath locking sleeve.

17. A method of dilating a sheath comprising:
providing a radially expandable sheath including:
a continuous inner layer defining a central lumen therethrough, the inner layer having at least one folded portion; and
tubular strain relief layer provided over the inner layer positioned at a proximal end of the sheath and extending along at least a portion of a length of the sheath, wherein at least a portion of the strain relief layer is configured to locally expand from an unexpanded configuration at a first diameter to an expanded configuration at a second, larger, diameter in response to an outwardly directed radial force exerted on the central lumen, and then locally contract at least partially back to the unexpanded configuration as the outwardly directed radial force is removed from the central lumen;
providing a dilator for expanding at least a portion of the sheath, the dilator including:
a dilator shaft sized and configured to be received within the central lumen of the expandable sheath;
a knob coupled to a proximal end of the dilator shaft;
a dilator hub rotatably coupled to the knob;
a pin coupling the dilator hub and the dilator shaft and preventing rotational movement therebetween;
coupling the dilator to the sheath;
rotating the knob in a first direction causing the dilator shaft to engage the knob and resulting in a corresponding axial movement of the dilator shaft in a distal direction along a longitudinal axis of the dilator thereby increasing a length of the dilator shaft received within the central lumen of the sheath;
advancing the dilator shaft through a portion of the central lumen of the sheath corresponding to the strain relief layer such that the dilator shaft exerts an outwardly directed radial force against the central lumen and causes the inner layer and the strain relief layer proximate the dilator shaft to locally expand from an unexpanded configuration to an expanded configuration;
rotating the knob in a second direction opposite to the first direction, causing the dilator shaft to engage the knob and resulting in a corresponding axial movement of the dilator shaft in a proximal direction along the longitudinal axis of the dilator thereby reducing a length of the dilator shaft received within the central lumen of the sheath and removing the outwardly directed radial force exerted on the central lumen thereby causing the inner layer and the strain relief layer to locally contract at least partially back to the unexpanded configuration;
uncoupling the dilator from the sheath; and
removing the dilator from the sheath.

18. The method of claim 17, wherein the dilator hub includes a locking channel sized and configured to couple the dilator hub to the sheath, the locking channel including a guide portion that extends between an opening on a distal end surface of the dilator hub and a locking portion of the locking channel, the locking portion extending in a direction circumferentially around the dilator hub,
wherein the sheath includes a sheath locking sleeve provided at a proximal end of the sheath, the sheath locking sleeve comprising a sleeve body having a proximal end and a distal end and defining a central lumen extending longitudinally between the proximal end and the distal end, a guide disposed on an outer surface of the sleeve body,
wherein coupling the dilator to the sheath includes:
advancing a distal end of the dilator shaft at least partially within the central lumen of the sheath;
positioning the dilator hub adjacent a proximal end of the sheath locking sleeve such that the guide projecting from an outer surface of the sheath locking sleeve is received within a locking channel opening on the dilator hub; and
rotating the dilator hub in a first direction with respect to the locking sleeve to move the guide along the locking channel into a locked position.

19. The method of claim 18, wherein movement of the guide along the locking channel into a locked position includes:
movement of the guide along a guide portion of the locking channel toward a locking portion of the locking channel, where the guide portion of the locking channel extends in a direction from the distal end of the dilator hub axially towards the proximal end of the dilator hub and the locking portion extends circumferentially around the dilator hub;
wherein further rotation of the dilator hub directs the guide into the locking portion of the locking channel, the locking portion configured to securely engage the guide and fix an axial position of the dilator hub with respect to the sheath locking sleeve.

20. The method of claim 17, wherein the knob includes a threaded central lumen extending at least partially therethrough and the dilator shaft includes a threaded outer surface received within and threadingly coupled to threaded central lumen of the knob,
wherein rotating the knob causes the threaded outer surface of the dilator shaft to threadingly engage the threaded central lumen of the knob resulting in a corresponding axial movement of the dilator shaft in a direction along the longitudinal axis of the dilator,
wherein the pin extends from the dilator hub through a slot extending radially through the dilator shaft and engagement between the pin and the slot restricts rotational movement between the dilator hub and the dilator shaft,
wherein rotating the knob causes the pin to slidingly engage the slot guiding axial movement of the dilator shaft, and limiting rotational movement of the dilator shaft with respect to the dilator hub.
